(19) United States
(12) Patent Application Publication
(10) Pub. No.: US 2010/0292241 A1
(43) Pub. Date: Nov. 18, 2010

(54) 3,5-SUBSTITUTED-1,3-OXAZOLIDIN-2-ONE DERIVATIVES

(75) Inventors: Edward Brnardic, Lansdale, PA (US); Mark Fraley, North Wales, PA (US); Mark Layton, Harleysville, PA (US); Robert Garbaccio, Lansdale, PA (US)

Correspondence Address: MERCK, P O BOX 2000, RAHWAY, NJ, 07065-0907, US

(73) Assignee: Merck Sharp & Dohme Corp., Rahway, NJ (US)

(21) Appl. No.: 12/863,636

(22) PCT Filed: Jan. 14, 2009
(86) PCT No.: PCT/US09/30884

§ 371 Date: Jul. 20, 2010

Related U.S. Application Data
(63) Continuation-in-part of application No. 61/062,233, filed on Jan. 24, 2008.

(51) Int. Cl. A61K 31/4985; A61K 31/5025; A61K 31/519; A61K 31/517; A61K 31/4365; A61K 31/437; A61K 31/4725; A61K 31/4709; A61K 31/4439; A61K 31/428; A61K 31/425; A61K 31/421; C07D 487/04; C07D 471/04; C07D 495/04; C07D 215/38; C07D 413/14; C07D 413/10; C07D 263/04; A61P 25/18

(52) U.S. Cl. 514/249; 514/248; 514/260.1; 514/266.2; 514/300; 514/301; 514/303; 514/309; 514/314; 514/340; 514/367; 514/372; 514/374; 514/376; 544/235; 544/236; 544/237; 544/278; 544/284; 544/354; 546/113; 546/114; 546/119; 546/141; 546/143; 546/171; 546/271.4; 548/178; 548/214; 548/229; 548/232

(57) ABSTRACT

The present invention is directed to 3,5-disubstituted-1,3-oxazolidin-2-one derivatives which are potentiators of metabotropic glutamate receptors, including the mGluR2 receptor, and which are useful in the treatment or prevention of neuro-ological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### BACKGROUND OF THE INVENTION

The excitatory amino acid L-glutamate (sometimes referred to herein simply as glutamate) through its many receptors mediates most of the excitatory neurotransmission within the mammalian central nervous system (CNS). The excitatory amino acids, including glutamate, are of great physiological importance, playing a role in a variety of physiological processes, such as long-term potentiation (learning and memory), the development of synaptic plasticity, motor control, respiration, cardiovascular regulation, and sensory perception.

Glutamate acts via at least two distinct classes of receptors. One class is composed of the tonotropic glutamate (iGlu) receptors that act as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

The present invention relates to potentiators of mGlu receptors, in particular mGluR2 receptors. The mGluR receptors belong to the Type III G-protein coupled receptor (GPCR) superfamily. This superfamily of GPCR's including the calcium-sensing receptors, GABAB receptors and pheromone receptors, which are unique in that they are activated by binding of effectors to the amino-terminus portion of the receptor protein. The mGlu receptors are thought to mediate glutamate's demonstrated ability to modulate intracellular signal transduction pathways. Ozawa, Kamiya and Tsuzuski, Prog. Neurobio., 54, 581 (1998). They have been demonstrated to be localized both pre- and post-synaptically where they can regulate neurotransmitter release, either glutamate or other neurotransmitters, or modify the post-synaptic response of neurotransmitters, respectively.

At present, there are eight distinct mGlu receptors that have been positively identified, cloned, and their sequences reported. These are further subdivided based on their amino acid sequence homology, their ability to effect certain signal transduction mechanisms, and their known pharmacological properties. Ozawa, Kamiya and Tsuzuski, Prog. Neurobio., 54, 581 (1998). For instance, the Group I mGluR receptors, which include the mGlulR and mGlu5R, are known to activate phospholipase C (PLC) via Gaq-proteins thereby resulting in the increased hydrolysis of phosphoinositides and intracellular calcium mobilization. There are several compounds that are reported to activate the Group I mGlu receptors including DHPG, (R/S)-3,5-dihydroxyphenylglycine. Schoepp, Goldworthy, Johnson, Salhoff and Baker, J. Neurochem., 63, 769 (1994); Ito, et al., keurorep., 3, 1013 (1992). The Group II mGlu receptors consist of the two distinct receptors, mGluR2 and mGluR3 receptors. Both have been found to be negatively coupled to adenylate cyclase via activation of Gai-protein. These receptors can be activated by a selective compound such as 1S,2S,SR,6S-2 aminobicyclo[3.1.0]hexane-2,6-dicarboxylate. Monn, et al., J. Med. Chem., 40, 528 (1997); Schoepp, et al., Neuropharmacol., 36, 1 (1997). This activitation leads to inhibition of glutamate release in the synapse (Cartmell et al, J Neurochem 75, 889 (2000)). Similarly, the Group III mGlu receptors, including mGluR4, mGluR6, mGluR7 and mGluR8, are negatively coupled to adenylate cyclase via Gai and are potently activated by L-AP4 (L-(+)-2-amino-4-phosphonobutyric acid). Schoepp, Neurochem. Int., 24, 439 (1994).

Nonselective mGluR2/mGluR3 receptor agonists (Monn, et al., J. Med. Chem., 43, 4893, (2000)) have shown efficacy in numerous animal models of anxiety and psychosis as well as human clinical trials in schizophrenia patients (Patil et al, Nature Medicine, 13, 1102 (2007)). Recent reports indicate that mGluR2 but not the mGluR3 receptor mediates the actions of the dual mGluR2/mGluR3 agonist LY379268 in mouse models predictive of antipsychotic activity. (Woolley et al, Psycopharmacology, DOI 10.1007/s00213-007-0974-x (2007)) Additionally, recent animal studies demonstrate that selective potentiation of the mGluR2 receptor has similar effects to such non-selective agonists (Galici et al, Journal of Pharmacology and Experimental Therapeutics, 315, 1181 (2005) suggesting an alternative strategy concerning the discovery of selective, positive allosteric modulators (PAM's or allosteric potentiators) of mGluR2 (Johnson et al, J. Med. Chem. 46, 3189, (2003); Pinkerton et al., J. Med. Chem., 47, 4595 (2004). These potentiators act by enabling the receptor to produce a maximal response to endogenous glutamate. Such allosteric potentiators do not bind at the glutamate binding site also known as the “orthosteric site”, and may benefit by binding to a site other than the highly conserved orthosteric site. A potential advantage to this approach includes the opportunity to have a distinct pharmacological profile by enhancing the activity of the endogenous ligand upon its binding to the orthosteric site. The pharmacological distinctions include the potential for pharmacological specificity between related receptor types that share the same endogenous ligand. In addition, positive allosteric modulators of mGluR2 have been shown to potentiate the response of mGluR2 agonists such as LY379268 (Johnson et. Al. Biochemical Soc. Trans. 32, 881 (2004) and this represents an alternative strategy for treatment using mGluR2 selective PAMs.

It has become increasingly clear that there is a link between modulation of excitatory amino acid receptors, including the glutamatergic system, through changes in glutamate release or alteration in postsynaptic receptor activation, and a variety of neurological and psychiatric disorders. e.g. Monaghan, Bridges and Cotman, Ann. Rev. Pharmacol. Toxicol., 29, 365-402 (1989); Schoepp and Sacann, Neurobio. Aging, 15, 261-263 (1994); Meldrum and Garthwaite, Tr. Pharmacol. Sci., 11, 379-387 (1990). The medical consequences of such glutamate dysfunction makes the abatement of these neurological processes an important therapeutic goal.

### SUMMARY OF THE INVENTION

The present invention is directed to 3,5-disubstituted-1,3-oxazolidin-2-one derivatives which are potentiators of metabotropic glutamate receptors, including the mGluR2 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### DETAILED DESCRIPTION OF THE INVENTION

The invention encompasses a first genus of compounds of Formula I

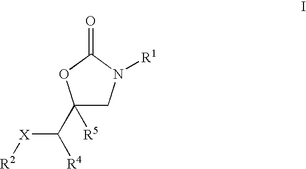

or a pharmaceutically acceptable salt thereof, wherein:
X is selected from the group consisting of —CH₂—, —O— and —N(R)—;
R¹ is selected from the group consisting of: aryl, —C₁₋₄alkylene-aryl, heteroaryl and —C₁₋₄alkylene-heteroaryl, wherein said aryl, heteroaryl, the aryl portion of —C₁₋₄alkylene-aryl and the heteroaryl portion of —C₁₋₄alkylene-heteroaryl are optionally substituted from one up to the maximum number of substitutable positions with R³;
R² is selected from the group consisting of: C₂₋₈alkyl, C₂₋₈alkenyl, C₂₋₈alkynyl, C₃₋₆cycloalkyl, aryl, —C₁₋₄alkylene-aryl, heteroaryl and —C₁₋₄alkylene-heteroaryl, wherein said C₁₋₈alkyl, C₂₋₈alkenyl, C₂₋₈alkynyl and C₃₋₆cycloalkyl are optionally substituted with 1 to 3 hydroxy groups and wherein said aryl, heteroaryl, the aryl portion of —C₁₋₄alkylene-aryl and the heteroaryl portion of —C₁₋₄alkylene-heteroaryl, are optionally substituted from one up to the maximum number of substitutable positions with R³;
each R³ is independently selected from the group consisting of
   (1) halo,
   (2) C₁₋₈alkyl, optionally substituted with oxo,
   (3) C₂₋₆alkenyl,
   (4) C₂₋₆alkynyl,
   (5) C₁₋₆haloalkyl,
   (6) C₁₋₆hydroxyalkyl,
   (7) C₃₋₆cycloalkyl,
   (8) C₁₋₆alkoxy,
   (9) C₁₋₆haloalkoxy,
   (10) —CN,
   (11) —C₁₋₄alkylene-CN,
   (12) -OH,
   (13) —C(O)—O—C₁₋₄alkyl,
   (14) —C(O)—C₁₋₄alkyl,
   (15) —C₁₋₄alkylene-C(O)—O—C₁₋₄alkyl,
   (16) —N(R)₂,
   (17) —C(O)—N(R)₂,
   (18) —S(O)ₖ—C₁₋₄alkyl, wherein k is 0, 1 or 2,
   (19) -aryl,
   (20) -heteroaryl,
   (21) —C(O)-aryl,
   (22) —N(R)-aryl,
   (23) benzyl,
   (24) benzyloxy,
   (25) —CO₂H,
   (26) —SH,
   (27) —SO₂N(R)R,
   (28) —N(R)C(O)N(R)R,
   (29) —N(R)C(O)C₁₋₄alkyl and
   (30) —N(R)SO₂N(R)R,
      or two R³ substituents on adjacent atoms may be joined together with the atoms to which they are attached to fowl a 5- or 6-membered saturated or partially unsaturated monocyclic ring optionally containing 1 or 2 heteroatoms selected from O, S and N, said ring optionally substituted with oxo or 1 to 3 halo groups, or both, and said ring optionally fused with a benzo group;
R⁴ and R⁵ are independently selected from the group consisting of: H and C₁₋₄alkyl, and
each R is independently selected from the group consisting of H and C₁₋₄alkyl.

Within the first genus, the invention encompasses a first subgenus of compounds of Formula I wherein
X is CH₂ and
R² is hexyl.

Also within the first genus, the invention encompasses a second subgenus of compounds of Formula I wherein
R² is phenyl, optionally substituted with one to five substituents R³.

The invention also encompasses a second genus of compounds of Formula la

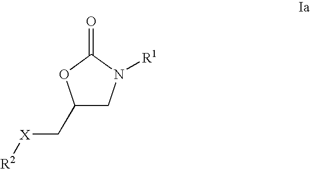

or a pharmaceutically acceptable salt thereof, wherein:
X is selected from the group consisting of: —CH₂— and —O—;
R¹ is selected from the group consisting of aryl, benzyl and heteroaryl, wherein said aryl, heteroaryl, and the phenyl portion of benzyl are optionally substituted from one up to the maximum number of substitutable positions with R³;
R² is selected from the group consisting of: C₂₋₈alkyl, C₂₋₈alkenyl, C₂₋₈alkynyl, C₃₋₆cycloalkyl and

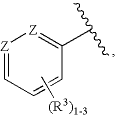

wherein said C₁₋₈alkyl, C₂₋₈alkenyl, C₂₋₈alkynyl and C₃₋₆cycloalkyl are optionally substituted with 1 to 3 hydroxy groups and each Z is independently CH or N;
each R³ is independently selected from the group consisting of
   (1) halo,
   (2) C₁₋₈alkyl, optionally substituted with oxo,
   (3) C₂₋₆alkenyl,
   (4) C₂₋₆alkynyl,
   (5) C₁₋₆haloalkyl,
   (6) C₁₋₆hydroxyalkyl,
   (7) C₃₋₆cycloalkyl,
   (8) C₁₋₆alkoxy,
   (9) C₁₋₆haloalkoxy,
   (10) —CN,
   (11) —C₁₋₄alkylene-CN,
   (12) —OH,
   (13) —C(O)—O—C₁₋₄alkyl,
   (14) —C(O)—C₁₋₄alkyl,
   (15) —C₁₋₄alkylene-C(O)—O—C₁₋₄alkyl,
   (16) —N(R)₂,
   (17) —C(O)—N(R)₂,
   (18) —S(O)ₖ—C₁₋₄alkyl, wherein k is 0, 1 or 2,
   (19) -aryl,
   (20) -heteroaryl,
   (21) —C(O)-aryl,
   (22) —N(R)-aryl,
   (23) benzyl,
   (24) benzyloxy,
   (25) —CO₂H,
   (26) —SH,
   (27) —SO₂N(R)R,
   (28) —N(R)C(O)N(R)R,
   (29) —N(R)C(O)C₁₋₄alkyl and
   (30) —N(R)SO₂N(R)R,
or two R³ substituents on adjacent atoms may be joined together with the atoms to which they are attached to form a 5- or 6-membered saturated or partially unsaturated monocyclic ring optionally containing 1 or 2 heteroatoms selected from O, S and N, said ring optionally substituted with oxo or 1 to 3 halo groups, or both, and said ring optionally fused with a benzo group; and
each R is independently selected from the group consisting of: H and C

Within the second genus, the inventions encompasses a third subgenus of compounds of Formula Ia wherein
X is —CH₂— and
R² is C₂₋₈alkyl, optionally substituted with hydroxy.

Within the third sub-genus, the invention encompasses a first class of compound of Formula Ia wherein R² is n-butyl.

Also within the second genus, the invention encompasses a fourth sub-genus of compounds of Formula Ia wherein
X is —O— and
R² is

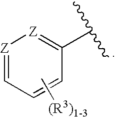

Within fourth sub-genus, the invention encompasses a second class of compounds of Formula Ia wherein each Z is CH or N.

Within the second class, the invention encompasses a sub-class of compounds of Formula Ia wherein one R³ is present attached at the para position.

Within the sub-class, the invention encompasses a group of compounds of Formula Ia wherein R³ is t-butyl.

Also within the second genus, the invention encompasses a fifth sub-genus of compounds of Formula Ia wherein R¹ is selected from the group consisting of: phenyl, naphthyl, thienyl, indolyl, benzothienyl, quinolinyl, benzothiazolyl, isoquinolinyl, pyrazolyl, indazolyl, furanyl, benzofuranyl, thiazolyl, pyrrolopyridinyl, isothiazolyl, pyrazolopyradazinyl, quinoxalinyl, quinazolinyl, thienopyridinyl, thienopyrimidinyl, cinnolinyl and triazolopyridinyl, each optionally substituted from one up to the maximum number of substitutable positions with R³.

Also within the second genus, the invention encompasses a sixth sub-genus of compounds of Formula Ia wherein R¹ is benzyl, wherein the phenyl portion of benzyl is optionally substituted with one to five substituents R³.

Also within the second genus, the invention encompasses a seventh sub-genus of compounds of Formula Ia wherein R¹ is aryl substituted with two R³ substituents on adjacent atoms that are joined together with the atoms to which they are attached to form a 5- or 6-membered saturated or partially unsaturated monocyclic ring optionally containing 1 or 2 heteroatoms selected from O, S and N, said ring optionally substituted with oxo or 1 to 3 halo groups, or both, and said ring optionally fused with a benzo group.

Within seventh sub-genus, the invention encompasses a third class of compounds of Formula Ia wherein R¹ is selected from the group consisting of 1,3-benzodioxolyl, 9-oxo-9H-fluorenyl, 2,3-dihydro-1,4-benzodioxinyl, 1-oxo-2,3-dihydro-1H-indenyl, 3-oxo-2,3-dihydro-1H-indenyl, 5-oxo-5,6,7,8-tetrahydronapthalenyl, 2,3-dihydro-1-benzofuranyl, 3,4-dihydroisoquinolin-1(2H)-onyl and phthalazin-1(2H)-onyl.

The invention also encompasses a compound selected from the group consisting of:
(5S)-5-[(4-tert-butylphenoxy)methyl]-3-thieno[2,3-c]pyridin-3-yl-1,3-oxazolidin-2-one;
5-hexyl-3-phenyl-1,3-oxazolidin-2-one;
5-hexyl-3-(3-methoxyphenyl)-1,3-oxazolidin-2-one;
3-biphenyl-3-yl-5-hexyl-1,3-oxazolidin-2-one;
5-hexyl-3-(3-methylphenyl)-1,3-oxazolidin-2-one;
3-(3,5-dimethylphenyl)-5-hexyl-1,3-oxazolidin-2-one;
3-[4-(dimethylamino)phenyl]-5-hexyl-1,3-oxazolidin-2-one;
5-hexyl-3-(4-methoxyphenyl)-1,3-oxazolidin-2-one,
3-(4-ethoxyphenyl)-5-hexyl-1,3-oxazolidin-2-one;
5-hexyl-3-[-4-(methylthio)phenyl]-1,3-oxazolidin-2-one;
3-(4-benzoylphenyl)-5-hexyl-1,3-oxazolidin-2-one;
3-(4-acetylphenyl)-5-hexyl-1,3-oxazolidin-2-one;
3-(4-tert-butylphenyl)-5-hexyl-1,3-oxazolidin-2-one;
5-hexyl-3-(4-methylphenyl)-1,3-oxazolidin-2-one;
3-(2-fluorophenyl)-5-hexyl-1,3-oxazolidin-2-one;
3-(3,4-difluorophenyl)-5-hexyl-1,3-oxazolidin-2-one;
5-hexyl-3-(2,4,5-trifluorophenyl)-1,3-oxazolidin-2-one;
3-(3-fluorophenyl)-5-hexyl-1,3-oxazolidin-2-one;
3-(2,4-difluorophenyl)-5-hexyl-1,3-oxazolidin-2-one;
3-(4-fluorophenyl)-5-hexyl-1,3-oxazolidin-2-one;
3-(4-fluoro-3-methylphenyl)-5-hexyl-1,3-oxazolidin-2-one;
3-(2,5-difluorophenyl)-5-hexyl-1,3-oxazolidin-2-one;
5-hexyl-3-[3-(trifluoromethyl)phenyl]-1,3-oxazolidin-2-one;
3-[3,5-bis(trifluoromethyl)phenyl]-5-hexyl-1,3-oxazolidin-2-one;
3-(3-chlorophenyl)-5-hexyl-1,3-oxazolidin-2-one;
3-(4-chlorophenyl)-5-hexyl-1,3-oxazolidin-2-one;
2-(5-hexyl-2-oxo-1,3-oxazolidin-3-yl)benzonitrile;
3-(5-hexyl-2-oxo-1,3-oxazolidin-3-yl)benzonitrile;
4-(5-hexyl-2-oxo-1,3-oxazolidin-3-yl)benzonitrile;
5-hexyl-3-(3-hydroxyphenyl)-1,3-oxazolidin-2-one;
5-hexyl-3-(4-hydroxyphenyl)-1,3-oxazolidin-2-one;
5-hexyl-3-(2-naphthyl)-1,3-oxazolidin-2-one;
5-hexyl-3-(2-thienyl)-1,3-oxazolidin-2-one;
5-hexyl-3-(3-thienyl)-1,3-oxazolidin-2-one;
5-hexyl-3-(1H-indol-5-yl)-1,3-oxazolidin-2-one;
3-(1,3-benzodioxol-5-yl)-5-hexyl-1,3-oxazolidin-2-one;
3-(3-aminophenyl)-5-hexyl-1,3-oxazolidin-2-one;
3-(4-aminophenyl)-5-hexyl-1,3-oxazolidin-2-one;
4-(5-hexyl-2-oxo-1,3-oxazolidin-3-yl)benzamide;
3-(3,4-dimethoxyphenyl)-5-hexyl-1,3-oxazolidin-2-one;
3-(2,6-difluorophenyl)-5-hexyl-1,3-oxazolidin-2-one;
3-(3,5-difluorophenyl)-5-hexyl-1,3-oxazolidin-2-one;
5-hexyl-3-(9-oxo-9H-fluoren-2-yl)-1,3-oxazolidin-2-one;
3-(3-fluoro-4-methoxyphenyl)-5-hexyl-1,3-oxazolidin-2-one;
3-(3-fluoro-4-hydroxyphenyl)-5-hexyl-1,3-oxazolidin-2-one;
5-hexyl-3-(4-propylphenyl)-1,3-oxazolidin-2-one;
5-hexyl-3-(2,3,4-trifluorophenyl)-1,3-oxazolidin-2-one;
methyl 4-(5-hexyl-2-oxo-1,3-oxazolidin-3-yl)benzoate;
3-(3-fluoro-4-methylphenyl)-5-hexyl-1,3-oxazolidin-2-one;
ethyl[4-(5-hexyl-2-oxo-1,3-oxazolidin-3-yl)phenyl]acetate;
methyl 3-(5-hexyl-2-oxo-1,3-oxazolidin-3-yl)benzoate;
3-(4-chloro-3-methylphenyl)-5-hexyl-1,3-oxazolidin-2-one;
3-(1-benzothien-3-yl)-5-hexyl-1,3-oxazolidin-2-one;
5-hexyl-3-quinolin-6-yl-1,3-oxazolidin-2-one;
5-hexyl-3-(4-isopropylphenyl)-1,3-oxazolidin-2-one;
3-(2,3-dihydro-1,4-benzodioxin-6-yl)-5-hexyl-1,3-oxazolidin-2-one;
3-(2-fluoro-5-methylphenyl)-5-hexyl-1,3-oxazolidin-2-one;
5-hexyl-3-[4-(trifluoromethoxy)phenyl]-1,3-oxazolidin-2-one;
3-(3,4-dichlorophenyl)-5-hexyl-1,3-oxazolidin-2-one;
2-chloro-4-(5-hexyl-2-oxo-1,3-oxazolidin-3-yl)benzonitrile;
3-[4-fluoro-3-(trifluoromethyl)phenyl]-5-hexyl-1,3-oxazolidin-2-one;
3-(2-fluoro-4-iodophenyl)-5-hexyl-1,3-oxazolidin-2-one;
3-[3-(dimethylamino)phenyl]-5-hexyl-1,3-oxazolidin-2-one;
3-(2-fluorobiphenyl-4-yl)-5-hexyl-1,3-oxazolidin-2-one;
3-(4-benzylphenyl)-5-hexyl-1,3-oxazolidin-2-one;
5-hexyl-3-[4-(1H-imidazol-1-yl)phenyl]-1,3-oxazolidin-2-one;
3-(2,3-difluorophenyl)-5-hexyl-1,3-oxazolidin-2-one;
5-hexyl-3-(3-isopropoxyphenyl)-1,3-oxazolidin-2-one;
3-(4-chloro-3-methoxyphenyl)-5-hexyl-1,3-oxazolidin-2-one;
5-hexyl-3-(2-methylquinolin-6-yl)-1,3-oxazolidin-2-one;
5-hexyl-3-(1-oxo-2,3-dihydro-1H-inden-5-yl)-1,3-oxazolidin-2-one;
5-hexyl-3-(5-pyridin-2-yl-2-thienyl)-1,3-oxazolidin-2-one;
3-[3-(benzyloxy)phenyl]-5-hexyl-1,3-oxazolidin-2-one;
5-hexyl-3-(1-methyl-1H-indol-5-yl)-1,3-oxazolidin-2-one;
3-[3-amino-5-(trifluoromethyl)phenyl]-5-hexyl-1,3-oxazolidin-2-one;
3-(2,2-difluoro-1,3-benzodioxol-5-yl)-5-hexyl-1,3-oxazolidin-2-one;
5-hexyl-3-(2,3,6-trifluorophenyl)-1,3-oxazolidin-2-one;
3-[3-(difluoromethoxy)phenyl]-5-hexyl-1,3-oxazolidin-2-one;
5-hexyl-3-(1H-indol-6-yl)-1,3-oxazolidin-2-one;
5-hexyl-3-(2-methyl-1,3-benzothiazol-5-yl)-1,3-oxazolidin-2-one;
5-hexyl-3-(3-isopropylphenyl)-1,3-oxazolidin-2-one;
5-hexyl-3-(4-methoxy-3-methylphenyl)-1,3-oxazolidin-2-one;
3-(3,5-difluoro-4-methoxyphenyl)-5-hexyl-1,3-oxazolidin-2-one;
3-(3,4-difluoro-5-methoxyphenyl)-5-hexyl-1,3-oxazolidin-2-one;
5-hexyl-3-[4-methoxy-3-(trifluoromethyl)phenyl]-1,3-oxazolidin-2-one;
5-hexyl-3-(1-hydroxyisoquinolin-7-yl)-1,3-oxazolidin-2-one;
5-hexyl-3-(3-oxo-2,3-dihydro-1H-inden-5-yl)-1,3-oxazolidin-2-one;
5-hexyl-3-(8-oxo-5,6,7,8-tetrahydronaphthalen-2-yl)-1,3-oxazolidin-2-one;
3-(2,3-dihydro -1-benzofuran-5-yl)-5-hexyl-1,3-oxazolidin-2-one;
5-hexyl-3-(5-methyl-1-phenyl-1H-pyrazol-4-yl)-1,3-oxazolidin-2-one;
5-hexyl-3-[4-(methylamino)phenyl]-1,3-oxazolidin-2-one;
3-(1-benzothien-5-yl)-5-hexyl-1,3-oxazolidin-2-one;
5-hexyl-3-[3-(1,1,2,2-tetrafluoroethoxy)phenyl]-1,3-oxazolidin-2-one;
5-hexyl-3-[3-(methylsulfonyl)phenyl]-1,3-oxazolidin-2-one;
5-hexyl-3-(1H-indazol-6-yl)-1,3-oxazolidin-2-one;
3-(1-benzofuran-5-yl)-5-hexyl-1,3-oxazolidin-2-one;
3-fluoro-5-(5-hexyl-2-oxo-1,3-oxazolidin-3-yl)benzonitrile;
5-hexyl-3-(5-oxo-5,6,7,8-tetrahydronaphthalen-2-yl)-1,3-oxazolidin-2-one;
6-(5-hexyl-2-oxo-1,3-oxazolidin-3-yl)-3,4-dihydroisoquinolin-1(2H)-one;
3-(1,3-benzothiazol-6-yl)-5-hexyl-1,3-oxazolidin-2-one;
5-hexyl-3-(5-methyl-3-thienyl)-1,3-oxazolidin-2-one;
5-hexyl-3-isoquinolin-6-yl-1,3-oxazolidin-2-one;
5-hexyl-3-[3-(methylamino)phenyl]-1,3-oxazolidin-2-one;
3-(3,5-dimethoxyphenyl)-5-hexyl-1,3-oxazolidin-2-one;
5-hexyl-3-(1,3-thiazol-4-yl)-1,3-oxazolidin-2-one;
2-fluoro-6-(5-hexyl-2-oxo-1,3-oxazolidin-3-yl)benzonitrile;
5-hexyl-3-(3-methoxy-4-methylphenyl)-1,3-oxazolidin-2-one;
[2-fluoro-4-(5-hexyl-2-oxo-1,3-oxazolidin-3-yl)phenyl]acetonitrile;
5-hexyl-3-(1H-pyrrolo[2,3-b]pyridin-3-yl)-1,3-oxazolidin-2-one;
5-hexyl-3-isothiazol-4-yl-1,3-oxazolidin-2-one;
5-hexyl-3-isothiazol-5-yl-1,3-oxazolidin-2-one;
3-(2-fluoro-3-methoxyphenyl)-5-hexyl-1,3-oxazolidin-2-one;
5-hexyl-3-(1-methyl-1H-pyrazol-4-yl)-1,3-oxazolidin-2-one;
5-hexyl-3-[4-(2-hydroxy-2-methylpropyl)phenyl]-1,3-oxazolidin-2-one;
5-hexyl-3-pyrazolo[1,5-b]pyridazin-3-yl-1,3-oxazolidin-2-one;
5-hexyl-3-(2-methoxyquinoxalin-6-yl)-1,3-oxazolidin-2-one;
5-hexyl-3-(2-methoxyquinazolin-6-yl)-1,3-oxazolidin-2-one;
3-(3-cyclopropylphenyl)-5-hexyl-1,3-oxazolidin-2-one;
3-(4-cyclopropylphenyl)-5-hexyl-1,3-oxazolidin-2-one;
5-hexyl-3-quinazolin-7-yl-1,3-oxazolidin-2-one;
5-hexyl-3-[2-(trifluoromethyl)quinolin-6-yl]-1,3-oxazolidin-2-one;
5-hexyl-3-quinazolin-6-yl-1,3-oxazolidin-2-one;
5-hexyl-3-thieno[2,3-c]pyridin-3-yl-1,3-oxazolidin-2-one;
5-hexyl-3-thieno[2,3-c]pyridin-2-yl-1,3-oxazolidin-2-one;
5-hexyl-3-(3-methoxyisoquinolin-7-yl)-1,3-oxazolidin-2-one;
5-hexyl-3-thieno[3,2-d]pyrimidin-6-yl-1,3-oxazolidin-2-one;
5-hexyl-3-(3-methoxycinnolin-7-yl)-1,3-oxazolidin-2-one;
5-hexyl-3-[4-(4H-1,2,4-triazol-3-yl)phenyl]-1,3-oxazolidin-2-one;
5-hexyl-3-[1,2,4]triazolo[1,5-a]pyridin-6-yl-1,3-oxazolidin-2-one;
7-(5-hexyl-2-oxo-1,3-oxazolidin-3-yl)phthalazin-1(2H)-one;
3-(2-cyclopropyl[1,2,4]triazolo[1,5-a]pyridin-6-yl)-5-hexyl-1,3-oxazolidin-2-one;
3-(4-amino-3-fluorophenyl)-5-hexyl-1,3-oxazolidin-2-one;
3-(4-cyclohexylphenyl)-5-hexyl-1,3-oxazolidin-2-one;
2-fluoro-4-(5-hexyl-2-oxo-1,3-oxazolidin-3-yl)benzonitrile;
3-(3-fluoro-5-methoxyphenyl)-5-hexyl-1,3-oxazolidin-2-one;
5-decyl-3-phenyl-1,3-oxazolidin-2-one;
5-butyl-3-phenyl-1,3-oxazolidin-2-one;
5-octyl-3-phenyl-1,3-oxazolidin-2-one;
5-but-3-en-1-yl-3-phenyl-1,3-oxazolidin-2-one;
5-(isobutoxymethyl)-3-phenyl-1,3-oxazolidin-2-one;
5-pentyl-3-phenyl-1,3-oxazolidin-2-one;
5-[(4-tert-butylphenoxy)methyl]-3-phenyl-1,3-oxazolidin-2-one;
5-{[(2-ethylhexyl)oxy]methyl}-3-phenyl-1,3-oxazolidin-2-one;
5-(butoxymethyl)-3-phenyl-1,3-oxazolidin-2-one;
(5R)-5-[2-(4-tert-butylphenyl)ethyl]-3-(3,4-dimethoxyphenyl)-1,3-oxazolidin-2-one;
(5R)-5-[2-(4-tert-butylphenyl)ethyl]-3-(4-chloro-3-methoxyphenyl)-1,3-oxazolidin-2-one;
3-{(5R)-5-[2-(4-tert-butylphenyl)ethyl]-2-oxo-1,3-oxazolidin-3-yl}benzonitrile;
(5R)-5-[2-(4-tert-butylphenyl)ethyl]-3-phenyl-1,3-oxazolidin-2-one;
(5R)-5-[2-(4-tert-butylphenyl)ethyl]-3-isothiazol-5-yl-1,3-oxazolidin-2-one;
(5R)-5-[2-(4-tert-butylphenyl)ethyl]-3-thieno[2,3-c]pyridin-2-yl-1,3-oxazolidin-2-one;
(5R)-5-[2-(4-tert-butylphenyl)ethyl]-3-thieno[2,3-c]pyridin-3-yl-1,3-oxazolidin-2-one;
5-[(4-tert-butylphenoxy)methyl]-3-(3,4-dimethoxyphenyl)-1,3-oxazolidin-2-one;
3-{5-[(4-tert-butylphenoxy)methyl]-2-oxo-1,3-oxazolidin-3-yl}benzonitrile;
5-[(4-tert-butylphenoxy)methyl]-3-(4-chloro-3-methoxyphenyl)-1,3-oxazolidin-2-one;
5-[(4-tert-butylphenoxy)methyl]-3-thieno[2,3-c]pyridin-2-yl-1,3-oxazolidin-2-one;
5-[(4-tert-butylphenoxy)methyl]-3-thieno[2,3-e]pyridin-3-yl-1,3-oxazolidin-2-one;
5-[(4-tert-butylphenoxy)methyl]-3-isothiazol-5-yl-1,3-oxazolidin-2-one;
5-[(4-tert-butylphenoxy)methyl]-3-phenyl-1,3-oxazolidin-2-one;
5-[(biphenyl-4-yloxy)methyl]-3-phenyl-1,3-oxazolidin-2-one;
5-[(3-ethylphenoxy)methyl]-3-phenyl-1,3-oxazolidin-2-one;
3-phenyl-5-[(5,6,7,8-tetrahydronaphthalen-2-yloxy)methyl]-1,3-oxazolidin-2-one;
5-[(4-ethylphenoxy)methyl]-3-phenyl-1,3-oxazolidin-2-one;
3-phenyl-5-[(4-propylphenoxy)methyl]-1,3-oxazolidin-2-one;
5-[(3-chloro-4-fluorophenoxy)methyl]-3-phenyl-1,3-oxazolidin-2-one;
5-[(4-isopropylphenoxy)methyl]-3-phenyl-1,3-oxazolidin-2-one;
3-phenyl-5-{[4-(trifluoromethyl)phenoxy]methyl}-1,3-oxazolidin-2-one;
5-{[4-(1,1-dimethylpropyl)phenoxy]methyl}-3-phenyl-1,3-oxazolidin-2-one;
5-[(3-chlorophenoxy)methyl]-3-phenyl-1,3-oxazolidin-2-one;
3-phenyl-5-[(3-propylphenoxy)methyl]-1,3-oxazolidin-2-one;
ethyl 4-[(2-oxo-3-phenyl-1,3-oxazolidin-5-yl)methoxy]benzoate;
5-[(4-bromophenoxy)methyl]-3-phenyl-1,3-oxazolidin-2-one;
3-phenyl-5-[(3,4,5-trimethylphenoxy)methyl]-1,3-oxazolidin-2-one;
5-[(2,3-dihydro-1H-inden-5-yloxy)methyl]-3-phenyl-1,3-oxazolidin-2-one;
5-[(3,5-dimethylphenoxy)methyl]-3-phenyl-1,3-oxazolidin-2-one;
5-[(4-ethoxyphenoxy)methyl]-3-phenyl-1,3-oxazolidin-2-one;
ethyl 3-[(2-oxo-3-phenyl-1,3-oxazolidin-5-yl)methoxy]benzoate;
5-[(3,5-dichlorophenoxy)methyl]-3-phenyl-1,3-oxazolidin-2-one;
3-phenyl-5-{[3-(trifluoromethyl)phenoxy]methyl}-1,3-oxazolidin-2-one;
3-phenyl-5-[(4-propoxyphenoxy)methyl]-1,3-oxazolidin-2-one;
3-phenyl-5-[(5,6,7,8-tetrahydronaphthalen-1-yloxy)methyl]-1,3-oxazolidin-2-one;
5-[(4-fluorophenoxy)methyl]-3-phenyl-1,3-oxazolidin-2-one;
5-[(biphenyl-3-yloxy)methyl]-3-phenyl-1,3-oxazolidin-2-one;
5-[(2,3-difluorophenoxy)methyl]-3-phenyl-1,3-oxazolidin-2-one;
5-[(4-benzoylphenoxy)methyl]-3-phenyl-1,3-oxazolidin-2-one;
5-[(4-methoxyphenoxy)methyl]-3-phenyl-1,3-oxazolidin-2-one;
3-phenyl-5-[(2-propylphenoxy)methyl]-1,3-oxazolidin-2-one;
5-{[3-(dimethylamino)phenoxy]methyl}-3-phenyl-1,3-oxazolidin-2-one;
5-[(1-benzofuran-6-yloxy)methyl]-3-phenyl-1,3-oxazolidin-2-one;
5-[(4-phenoxyphenoxy)methyl]-3-phenyl-1,3-oxazolidin-2-one;
5-[(3-methylphenoxy)methyl]-3-phenyl-1,3-oxazolidin-2-one;
5-[(3,5-difluorophenoxy)methyl]-3-phenyl-1,3-oxazolidin-2-one;
5-(phenoxymethyl)-3-phenyl-1,3-oxazolidin-2-one;
5-[(2-chlorophenoxy)methyl]-3-phenyl-1,3-oxazolidin-2-one;
5-[(3-methoxyphenoxy)methyl]-3-phenyl-1,3-oxazolidin-2-one;
5-{[3-(diethylamino)phenoxy]methyl}-3-phenyl-1,3-oxazolidin-2-one;
5-[(1-naphthyloxy)methyl]-3-phenyl-1,3-oxazolidin-2-one;
5-[(2,4-dichlorophenoxy)methyl]-3-phenyl-1,3-oxazolidin-2-one;
5-[(4-cyclopentylphenoxy)methyl]-3-phenyl-1,3-oxazolidin-2-one;
5-[(3-teat-butylphenoxy)methyl]-3-phenyl-1,3-oxazolidin-2-one;
5-[(3,4-dichlorophenoxy)methyl]-3-phenyl-1,3-oxazolidin-2-one;
5-[(3-ethynylphenoxy)methyl]-3-phenyl-1,3-oxazolidin-2-one;
3-phenyl-5-{[3-(trifluoromethoxy)phenoxy]methyl}-1,3-oxazolidin-2-one;
5-[(3,5-dimethoxyphenoxy)methyl]-3-phenyl-1,3-oxazolidin-2-one;
5-[(4-isopropyl-3-methylphenoxy)methyl]-3-phenyl-1,3-oxazolidin-2-one;
5-{[4-fluoro-3-(trifluoromethyl)phenoxy]methyl}-3-phenyl-1,3-oxazolidin-2-one;
5-[(3-chloro-4-methylphenoxy)methyl]-3-phenyl-1,3-oxazolidin-2-one;
3-phenyl-5-{(quinolin-5-yloxy)methyl]-1,3-oxazolidin-2-one;
5-[(4-bromo-2-fluorophenoxy)methyl]-3-phenyl-1,3-oxazolidin-2-one;
5-[(4-bromo-3-fluorophenoxy)methyl]-3-phenyl-1,3-oxazolidin-2-one;
5-[(4-tert-butyl-2-methylphenoxy)methyl]-3-phenyl-1,3-oxazolidin-2-one;
3-phenyl-5-{[4-(trifluoromethoxy)phenoxy]methyl}-1,3-oxazolidin-2-one;
5-[(3-aminophenoxy)methyl]-3-phenyl-1,3-oxazolidin-2-one;
5-{[4-(3-oxobutyl)phenoxy]methyl}-3-phenyl-1,3-oxazolidin-2-one;
5-[(4-chloro-3-fluorophenoxy)methyl]-3-phenyl-1,3-oxazolidin-2-one;
5-[(3-tert-butyl-4-methoxyphenoxy)methyl]-3-phenyl-1,3-oxazolidin-2-one;
5-{[3-fluoro-5-(trifluoromethyl)phenoxy]methyl}-3-phenyl-1,3-oxazolidin-2-one;
ethyl 3-{4-[(2-oxo-3-phenyl-1,3-oxazolidin-5-yl)methoxy]phenyl}propanoate;
5-[(1,3-benzodioxol-5-yloxy)methyl]-3-phenyl-1,3-oxazolidin-2-one;
5-[(4-chloro-2-fluorophenoxy)methyl]-3-phenyl-1,3-oxazolidin-2-one;
5-[(3-phenoxyphenoxy)methyl]-3-phenyl-1,3-oxazolidin-2-one;
5-[(4-chloro-2-methoxyphenoxy)methyl]-3-phenyl-1,3-oxazolidin-2-one;
methyl 3-chloro-4-[(2-oxo-3-phenyl-1,3-oxazolidin-5-yl)methoxy]benzoate;
5-[(2,5-difluorophenoxy)methyl]-3-phenyl-1,3-oxazolidin-2-one;
5-[(3-benzoylphenoxy)methyl]-3-phenyl-1,3-oxazolidin-2-one;
5-[(isoquinolin-7-yloxy)methyl]-3-phenyl-1,3-oxazolidin-2-one;
5-[(4-anilinophenoxy)methyl]-3-phenyl-1,3-oxazolidin-2-one;
3-phenyl-5-[(3,4,5-trifluorophenoxy)methyl]-1,3-oxazolidin-2-one;
5-{[2-chloro-3-(trifluoromethyl)phenoxy]methyl)-3-phenyl-1,3-oxazolidin-2-one;
5-[(2,4-difluorophenoxy)methyl]-3-phenyl-1,3-oxazolidin-2-one;
3-phenyl-5-[(3,4,-trimethoxyphenoxy)methyl]-1,3-oxazolidin-2-one;
5-[(2,5-dichlorophenoxy)methyl]-3-phenyl-1,3-oxazolidin-2-one;
3-phenyl-5-[(quinolin-8-yloxy)methyl]-1,3-oxazolidin-2-one;
5-[(4-acetyl-2-methylphenoxy)methyl]-3-phenyl-1,3-oxazolidin-2-one;
5-[(2-chloro-4-methoxyphenoxy)methyl]-3-phenyl-1,3-oxazolidin-2-one;
5-{[(3-oxo-2,3-dihydro-1-benzofuran-6-yl)oxy]methyl}-3-phenyl-1,3-oxazolidin-2-one;
3-phenyl-5-[(quinolin-6-yloxy)methyl]-1,3-oxazolidin-2-one;
3-benzyl-5-hexyl-1,3-oxazolidin-2-one;
3-benzyl-5-hexyl-1,3-oxazolidin-2-one;
5-hexyl-3-(2-methoxybenzyl)-1,3-oxazolidin-2-one;
5-hexyl-3-(3-methoxybenzyl)-1,3-oxazolidin-2-one;
5-hexyl-3-(4-methoxybenzyl)-1,3-oxazolidin-2-one;
2-[(5-hexyl-2-oxo-1,3-oxazolidin-3-yl)methyl]benzonitrile;
3-[(5-hexyl-2-oxo-1,3-oxazolidin-3-yl)methyl]benzonitrile;
4-[(5-hexyl-2-oxo-1,3-oxazolidin-3-yl)methyl]benzonitrile;
5-hexyl-3(2-methylbenzyl)-1,3-oxazolidin-2-one;
5-hexyl-3-(3-methylbenzyl)-1,3-oxazolidin-2-one;
5-hexyl-3-(4-methylbenzyl)-1,3-oxazolidin-2-one;
methyl 4-[(5-hexyl-2-oxo-1,3-oxazolidin-3-yl)methyl]benzoate;
3-(4-fluorobenzyl)-5-hexyl-1,3-oxazolidin-2-one;
5-hexyl-3-[3-(trifluoromethyl)benzyl]-1,3-oxazolidin-2-one;
3-(2-chlorobenzyl)-5-hexyl-1,3-oxazolidin-2-one;
3-(3-chlorobenzyl)-5-hexyl-1,3-oxazolidin-2-one;
3-(4-tert-butylbenzyl)-5-hexyl-1,3-oxazolidin-2-one;
3-(3-fluorobenzyl)-5-hexyl-1,3-oxazolidin-2-one;
5-hexyl-3-[3-(trifluoromethoxy)benzyl]-1,3-oxazolidin-2-one;
5-hexyl-3-[2-(trifluoromethoxy)benzyl]-1,3-oxazolidin-2-one;
5-hexyl-3-[4-(trifluoromethoxy)benzyl]-1,3-oxazolidin-2-one;
5-hexyl-3-[2-(trifluoromethyl)benzyl]-1,3-oxazolidin-2-one;
methyl 3-[(5-hexyl-2-oxo-1,3-oxazolidin-3-yl)methyl]benzoate;
3-(biphenyl-4-ylmethyl)-5-hexyl-1,3-oxazolidin-2-one;
3-(2-fluorobenzyl)-5-hexyl-1,3-oxazolidin-2-one;
5-hexyl-3-[4-(trifluoromethyl)benzyl]-1,3-oxazolidin-2-one;
3-(4-chlorobenzyl)-5-hexyl-1,3-oxazolidin-2-one;
5-{[(4-tert-butylbenzyl)(methyl)amino]methyl}-3-phenyl-1,3-oxazolidin-2-one;
5-{[(4-tert-butylbenzyl)amino]methyl}-3-phenyl-1,3-oxazolidin-2-one; and
5-{[benzyl(methyl)amino]methyl}-3-phenyl-1,3-oxazolidin-2-one,
   or a pharmaceutically acceptable salt of any of the foregoing compounds.

The invention also encompasses a pharmaceutical composition comprising a compound of Formula Ia in combination with a pharmaceutically acceptable carrier.

The invention also encompasses a method for treating a neurological or psychiatric disorder associated with glutamate dysfunction in a patient in need thereof comprising administering to the patient a therapeutically effective amount of a compound of Formula I. The invention also encompasses this method wherein the neurological or psychiatric disorder associated with glutamate dysfunction is schizophrenia.

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like.

“Alkylene” means a straight or branched chain of carbon atoms with a group substituted at both ends, such as —CH₂CH₂— and —CH₂CH₂CH₂—.

“Haloalkyl” means alkyl as defined above wherein one or more hydrogen atoms have been replaced by halogen atoms.

“Hydroxyalkyl” means alkyl as defined above wherein one or more hydrogen atoms have been replaced by hydroxy atoms.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic saturated carbocyclic rings, each of which having from 3 to 10 carbon atoms. A “fused analog” of cycloalkyl means a monocyclic rings fused to an aryl or heteroaryl group in which the point of attachment is on the non-aromatic portion. Examples of cycloalkyl and fused analogs thereof include cyclopropyl, cyclobutyl, cyclopentyl, cyclohexyl, cycloheptyl, tetrahydronaphthyl, decahydronaphthyl, indanyl, and the like.

“Alkoxy” means alkoxy groups of a straight or branched having the indicated number of carbon atoms. C₁₋₆alkoxy, for example, includes methoxy, ethoxy, propoxy, isopropoxy, and the like.

“Cycloalkoxy” means cycloalkyl as defined above bonded to an oxygen atom, such as cyclopropyloxy.

“Aryl” means mono- or bicyclic aromatic rings containing only carbon atoms. Examples of aryl include phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” means mono- or bicyclic aromatic rings with at least one ring containing a heteroatom selected from N, O and S, and each ring containing 5 or 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

“Halogen” and “halo” includes fluorine, chlorine, bromine and iodine.

The compounds of the present invention are potentiators of metabotropic glutamate (mGluR) receptor function, in particular they are potentiators of mGluR2 receptors. That is, the compounds of the present invention do not appear to bind at the glutamate recognition site on the mGluR receptor, but in the presence of glutamate or a glutamate agonist, the compounds of the present invention increase mGluR receptor response. The present potentiators are expected to have their effect at mGluR receptors by virtue of their ability to increase the response of such receptors to glutamate or glutamate agonists, enhancing the function of the receptors. It is recognized that the compounds of the present invention would be expected to increase the effectiveness of glutamate and glutamate agonists of the mGluR2 receptor. Thus, the potentiators of the present invention are expected to be useful in the treatment of various neurological and psychiatric disorders associated with glutamate dysfunction described to be treated herein and others that can be treated by such potentiators as are appreciated by those skilled in the art.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid fowl may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glutamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of Formula I are meant to also include a pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

The subject compounds are useful in a method of potentiating metabotorpic glutamate receptor activity in a patient such as a mammal in need of such inhibition comprising the administration of an effective amount of the compound. The present invention is directed to the use of the compounds disclosed herein as potentiators of metabotropic glutamate receptor activity. In addition to primates, especially humans, a variety of other mammals can be treated according to the method of the present invention.

The present invention is further directed to a method for the manufacture of a medicament for potentiating metabotropic glutamate receptor activity in humans and animals comprising combining a compound of the present invention with a pharmaceutical carrier or diluent.

The subject treated in the present methods is generally a mammal, preferably a human being, male or female, in whom potentiation of metabotropic glutamate receptor activity is desired. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with the disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of and or “administering a” compound should be understood to mean providing a compound of the invention or a prodrug of a compound of the invention to the individual in need of treatment.

The utility of the compounds in accordance with the present invention as inhibitors of metabotropic glutamate receptor activity, in particular mGluR2 activity, may be demonstrated by methodology known in the art. Inhibition constants are determined as follows. The compounds of the present invention may be tested in a fluorescence laser imaging plate reader (FLIPR) based assay. This assay is a common functional assay to monitor Ca²⁺ mobilization in whole cells expressing recombinant receptor coupled with a promiscuous G-protein. CHO dhfr− cells stably expressing recombinant human mGluR2 and Gct16 loaded with Fluo-4 AM (Invitrogen, Carlsbad Calif.) are treated with dose responses of compounds and the Ca²⁺ response is monitored on a FLIPR384 (Molecular Devices, Sunnydale Calif.) for agonist activity. The potentiation response is monitored after a subsequent addition of an EC20 concentration of glutamate (900 nM). The maximum calcium response at each concentration of compound for agonist or potentiation are plotted as dose responses and the curves are fitted with a four parameters logistic equation giving EC50 and Hill coefficient using the iterative non linear curve fitting software program.

The compounds of the present invention may also be tested in a [³⁵S]-GTPγS assay. The stimulation of [³⁵S]-GTPγS binding is a common functional assay to monitor Gαi-coupled receptor in native and recombinant receptor membrane preparation. Membrane from cells stably expressing hmGlu2 CHO-K1 (50 μg) are incubated in a 96 well plate for 1 hour in the presence of GTPγS³⁵ (0.05 nM), GDP (5 μM) and compounds. The reaction is stopped by rapid filtration over Unifilter GF/B plate (Packard, Bioscience, Meriden Conn.) using a 96-well cell harvester (Brandel Gaithersburg, Md.). The filter plates are counted using Topcount counter (Packard, Bioscience, Meriden Conn., USA). When compounds are evaluated as potentiators they are tested in the presence of glutamate (1 μM). The activation (agonist) or the potentiation of glutamate (potentiator) curves are fitted with a four parameters logistic equation giving EC₅₀ and Hill coefficient using the iterative non linear curve fitting software GraphPad (San Diego Calif., USA).

In particular, the compounds of the following examples demonstrated activity in potentiating the mGluR2 receptor in the FLIPR assay, generally with an EC₅₀ of less than about 10 μM. Preferred compounds within the present invention had activity in potentiating the mGluR2 receptor in the FLIPR and GTPγS assays with an EC₅₀ of less than about 1 μM. The following examples resulted in a minimum 1.8-fold potentiation of glutamate response in the presence of an EC20 concentration of glutamate (900 nM). Such results are indicative of the intrinsic activity of the compounds in use as potentiators of mGluR2 receptor activity.

Metabotropic glutamate receptors including the mGluR2 receptor have been implicated in a wide range of biological functions. This has suggested a potential role for these receptors in a variety of disease processes in humans or other species.

The compounds of the present invention have utility in treating, preventing, ameliorating, controlling or reducing the risk of a variety of neurological and psychiatric disorders associated with glutamate dysfunction, including one or more of the following conditions or diseases: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), autism, autism spectrum disorders, attention deficit/hyperactivity disorder, and conduct disorder.

Of the disorders above, the treatment of migraine, anxiety, schizophrenia, and epilepsy are of particular importance. In a preferred embodiment the present invention provides a method for treating migraine, comprising: administering to a patient in need thereof an effective amount of a compound of formula I. In another preferred embodiment the present invention provides a method for preventing or treating anxiety, comprising: administering to a patient in need thereof an effective amount of a compound of formula I. Particularly preferred anxiety disorders are generalized anxiety disorder, panic disorder, and obsessive compulsive disorder. In another preferred embodiment the present invention provides a method for treating schizophrenia, comprising: administering to a patient in need thereof an effective amount of a compound of formula I. In yet another preferred embodiment the present invention provides a method for treating epilepsy, comprising: administering to a patient in need thereof an effective amount of a compound of formula I.

Of the neurological and psychiatric disorders associated with glutamate dysfunction which are treated according to the present invention, the treatment of migraine, anxiety, schizophrenia, and epilepsy are particularly preferred. Particularly preferred anxiety disorders are generalized anxiety disorder, panic disorder, and obsessive compulsive disorder.

In an embodiment, the present invention provides a method for the treatment of schizophrenia comprising: administering to a patient in need thereof an effective amount of a compound of formula I or a pharmaceutical composition thereof. In one of the available sources of diagnostic tools, The Merck Manual (2006-2007), schizophrenia is characterized by psychosis (loss of contact with reality), hallucinations (false perceptions), delusions (false beliefs), disorganized speech and behavior, flattened affect (restricted range of emotions), cognitive deficits (impaired reasoning and problem solving), and occupational and social dysfunction. The skilled artisan will recognize that there are alternative nomenclatures, nosologies, and classification systems for neurological and psychiatric disorders, including migraine, and that these systems evolve with medical scientific progress

Thus, in an embodiment the present invention provides a method for treating migraine, comprising: administering to a patient in need thereof an effective amount of a compound of formula I or a pharmaceutical composition thereof. In one of the available sources of diagnostic tools, Dorland's Medical Dictionary (23'd Ed., 1982, W. B. Saunders Company, Philidelphia, Pa.), migraine is defined as a symptom complex of periodic headaches, usually temporal and unilateral, often with irritability, nausea, vomiting, constipation or diarrhea, and photophobia. As used herein the term “migraine” includes these periodic headaches, both temporal and unilateral, the associated irritability, nausea, vomiting, constipation or diarrhea, photophobia, and other associated symptoms. The skilled artisan will recognize that there are alternative nomenclatures, nosologies, and classification systems for neurological and psychiatric disorders, including migraine, and that these systems evolve with medical scientific progress.

In another embodiment the present invention provides a method for treating anxiety, comprising: administering to a patient in need thereof an effective amount of a compound of Formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including anxiety and related disorders. These include: panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified. As used herein the term “anxiety” includes treatment of those anxiety disorders and related disorder as described in the DSM-IV. The skilled artisan will recognize that there are alternative nomenclatures, nosologies, and classification systems for neurological and psychiatric disorders, and particular anxiety, and that these systems evolve with medical scientific progress. Thus, the term “anxiety” is intended to include like disorders that are described in other diagnostic sources.

In another embodiment the present invention provides a method for treating depression, comprising: administering to a patient in need thereof an effective amount of a compound of Formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including depression and related disorders. Depressive disorders include, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder. As used herein the term “depression” includes treatment of those depression disorders and related disorder as described in the DSM-IV.

In another embodiment the present invention provides a method for treating epilepsy, comprising: administering to a patient in need thereof an effective amount of a compound of Formula I or a pharmaceutical composition thereof. At present, there are several types and subtypes of seizures associated with epilepsy, including idiopathic, symptomatic, and cryptogenic. These epileptic seizures can be focal (partial) or generalized. They can also be simple or complex. Epilepsy is described in the art, such as Epilepsy: A comprehensive textbook. Ed. by Jerome Engel, Jr. and Timothy A. Pedley. (Lippincott-Raven, Philadelphia, 1997). At present, the International Classification of Diseases, Ninth Revision, (ICD-9) provides a diagnostic tool including epilepsy and related disorders. These include: generalized nonconvulsive epilepsy, generalized convulsive epilepsy, petit mal status epilepticus, grand mal status epilepticus, partial epilepsy with impairment of consciousness, partial epilepsy without impairment of consciousness, infantile spasms, epilepsy partialis continua, other forms of epilepsy, epilepsy, unspecified, NOS. As used herein the term “epilepsy” includes these all types and subtypes. The skilled artisan will recognize that there are alternative nomenclatures, nosologies, and classification systems for neurological and psychiatric disorders, including epilepsy, and that these systems evolve with medical scientific progress.

The subject compounds are further useful in a method for the prevention, treatment, control, amelioration, or reduction of risk of the diseases, disorders and conditions noted herein.

The subject compounds are further useful in a method for the prevention, treatment, control, amelioration, or reduction of risk of the aforementioned diseases, disorders and conditions in combination with other agents, including an mGluR agonist.

The term “potentiated amount” refers to an amount of an mGluR agonist, that is, the dosage of agonist which is effective in treating the neurological and psychiatric disorders described herein when administered in combination with an effective amount of a compound of the present invention. A potentiated amount is expected to be less than the amount that is required to provided the same effect when the mGluR agonist is administered without an effective amount of a compound of the present invention.

A potentiated amount can be readily determined by the attending diagnostician, as one skilled in the art, by the use of conventional techniques and by observing results obtained under analogous circumstances. In determining a potentiated amount, the dose of an mGluR agonist to be administered in combination with a compound of formula I, a number of factors are considered by the attending diagnostician, including, but not limited to: the mGluR agonist selected to be administered, including its potency and selectivity; the compound of formula Ito be coadministered; the species of mammal; its size, age, and general health; the specific disorder involved; the degree of involvement or the severity of the disorder; the response of the individual patient; the modes of administration; the bioavailability characteristics of the preparations administered; the dose regimens selected; the use of other concomitant medication; and other relevant circumstances.

A potentiated amount of an mGluR agonist to be administered in combination with an effective amount of a compound of formula I is expected to vary from about 0.1 milligram per kilogram of body weight per day (mg/kg/day) to about 100 mg/kg/day and is expected to be less than the amount that is required to provided the same effect when administered without an effective amount of a compound of formula I. Preferred amounts of a co-administered mGlu agonist are able to be determined by one skilled in the art.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also includes therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

The above combinations include combinations of a compound of the present invention not only with one other active compound, but also with two or more other active compounds.

Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICY, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The pharmaceutical composition and method of the present invention may further comprise other therapeutically active compounds as noted herein which are usually applied in the treatment of the above mentioned pathological conditions.

In the treatment, prevention, control, amelioration, or reduction of risk of conditions which require potentiation of metabotorpic glutamate receptor activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

When treating, preventing, controlling, ameliorating, or reducing the risk of neurological and psychiatric disorders associated with glutamate dysfunction or other diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made according to procedures known in the art or as illustrated herein. The compounds of the present invention can be prepared in a variety of fashions.

### General Schemes:

### General Scheme A:

Epichlorohydrin (A-1) can be reacted with a variety of phenols (A-2) to produce aryl glycidyl ethers (A-3) under basic conditions. Thermal reaction of glycidyl ethers (A-3) with ethyl carbamate in the presence of base provides the oxazolidinones (A-4). These oxazolidinones are functionalized at the open nitrogen position using copper promoted coupling to various aryl and heteroaryl halides (A-5) to provide final N-arylated oxazolidinones (A-6)

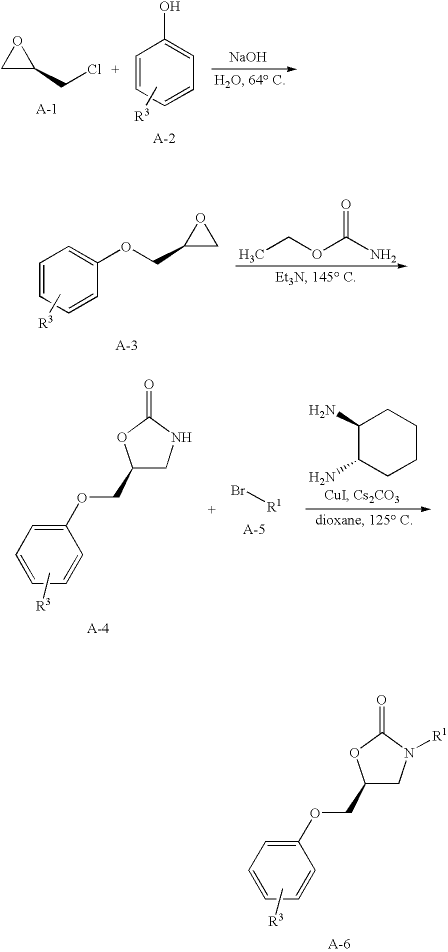

### General Scheme B:

In similar fashion, substituted epoxides (B-1) can be thermally reacted with ethyl carbamate in the presence of base provides the oxazolidinones (B-2). These oxazolidinones are functionalized at the open nitrogen position using copper promoted coupling to various aryl and heteroaryl halides (B-3) to provide final N-arylated oxazolidinones (B-4)

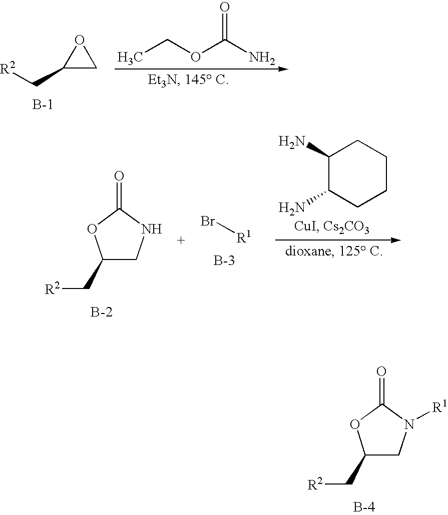

### General Scheme C:

An alternative sequence allows for late stage variation of the R2 substituent via Mitsunobu methodology. Benzyl glycidyl ether (C-1) is thermally condensed with R1 substituted carbamates to give oxazolidinones (C-2). Removal of the benzyl ether is effected by Pearlman's catalyst in EtOH to provide the alcohol (C-3). Mitsunobu reaction with various phenols yields final oxazolidinones (C-4).

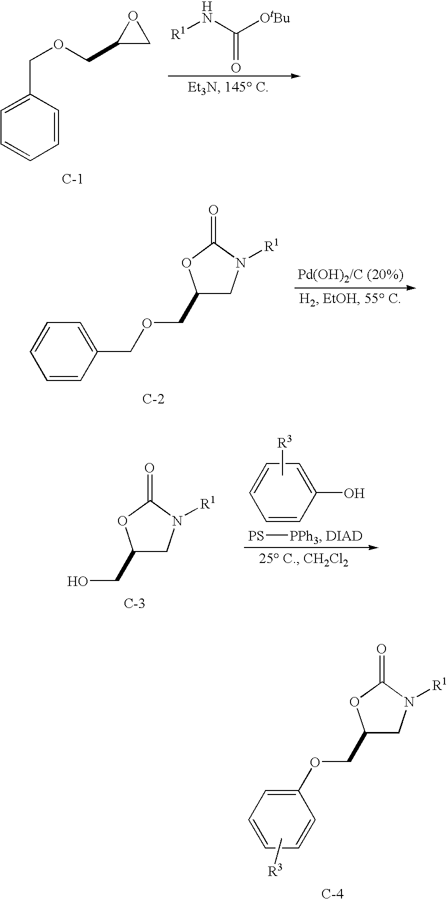

### General Scheme D:

An alternative functionalization of the oxazolidinones NH is also enabled by direct alkylation of D-1 using cesium carbonate and various electrophiles to give oxazolidinones D-2.

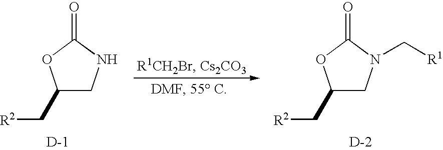

### General Scheme E:

In similar fashion, late-stage variation of the R2 substituent and introduction of amine side-chains can be effected according to Scheme E. Reaction of epibromohydrin (E-1) with isocyanates in the presence of SmI₃ directly provides bromo oxazolidinones (E-2). These electrophiles can be reacted with various amines or other nucleophiles to provide final oxazolidinones (E-3).

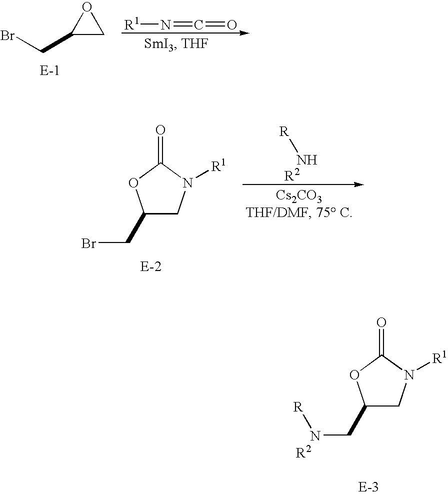

### Examples

### Example 1

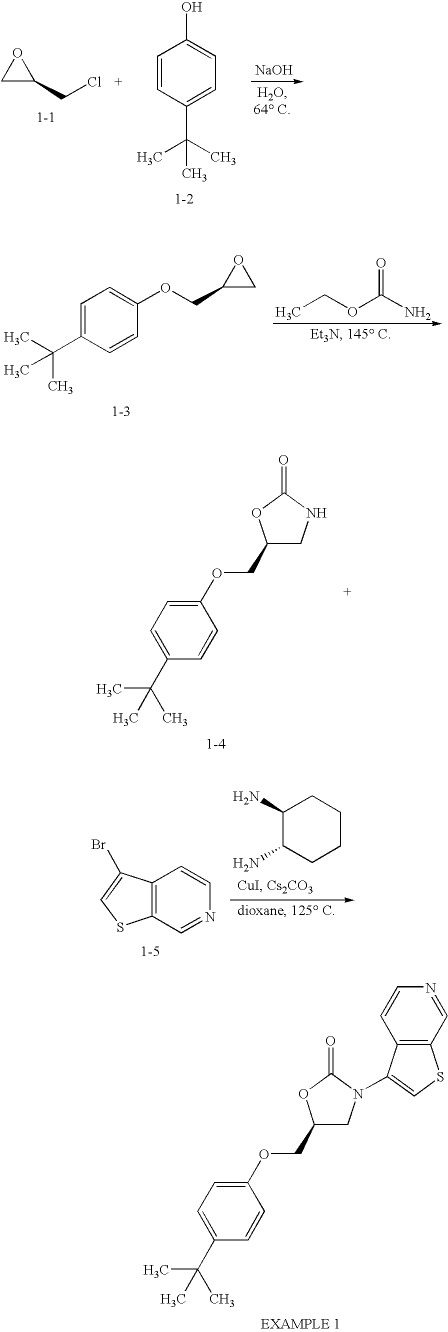

### (2S)-2-[(4-tert-butylphenoxy)methyl]oxirane (1-3)

To (R)-(−)-epichlorohydrin (1-1) (25 mL, 320 mmol, 2.0 eq) at 64° C. was added a warm solution of 4-tert-butylphenol (1-2) (24 grams, 160 mmol, 1.0 equiv) and sodium hydroxide (6.7 g, 170 mmol, 1.1 equiv) in water (50 mL) over 1 hour with vigorous stirring. The mixture was stirred at 64° C. for 7 hours and cooled to room temperature. The aqueous solution was extracted with diethyl ether and the combined organic extracts were washed with saturated aqueous sodium chloride solution, dried over sodium sulfate, and concentrated. The residue was purified by silica gel chromatography (0-100% ethyl acetate/hexanes) to yield (2S)-2-[(4-tert-butylphenoxy)methyl]oxirane (1-3) as a clear liquid. ¹H NMR (400 MHz, CDCl₃) δ 7.31 (d, 2H, J=8.8 Hz), 6.85 (d, 2H, J=8.8 Hz), 4.17 (m, 1H), 3.98 (m, 1H), 3.35 (m, 1H), 2.88 (m, 1H), 2.75 (m, 1H), 1.29 (s, 9H).

### (5S)-5-[(4-tert-butylphenoxy)methyl]-1,3-oxazolidin-2-one (1-4)

A mixture of (2S)-2-[(4-tert-butylphenoxy)methyl]oxirane (1-3) (15 g, 73 mmol, 1.0 equiv), ethyl carbamate (6.5 g, 73 mmol, 1.0 equiv) and triethyl amine (0.20 mL, 1.4 mmol, 0.020 equiv) was heated at 145° C. for 16 hours. The mixture was cooled and the product was purified by silica gel chromatography (0-100% ethyl acetate/hexanes) to yield (5S)-5-[(4-tert-butylphenoxy)methyl]-1,3-oxazolidin-2-one (1-4) as a white solid. ¹H NMR (400 MHz, CDCl₃) δ 7.31 (d, 2H, J=8.8 Hz), 6.85 (d, 2H, J=8.8 Hz), 6.38 (s, 1H), 4.95 (m, 1H), 4.13 (m, 2H), 3.77 (m, 1H), 3.60 (m, 1H), 1.29 (s, 9H). LRMS m/z (M+H) 250.3 found, 250.1 required.

### (5S)-5-[(4-tert-butylphenoxy)methyl]-3-thieno[2,3-c]pyridin-3-yl-1,3-oxazolidin-2-one

### Example 1

A mixture of (5S)-5-[(4-tert-butylphenoxy)methyl]-1,3-oxazolidin-2-one (1-4) (2.0 g, 8.0 mmol, 1.0 equiv), copper(I)iodide (1.8 g, 9.6 mmol, 1.2 equiv), trans-1,2-diaminocyclohexane (1.3 mL, 1.3 g, 11 mmol, 1.4 equiv), anhydrous cesium carbonate (3.9 g, 12 mmol, 1.5 equiv) and 3-bromothieno[2,3-c]pyridine (1-5) (1.9 g, 8.8 mmol, 1.1 equiv) in dioxane (60 mL) were heated at 125° C. in a sealed vial for 16 hours. The mixture was cooled, filtered and washed with dichloromethane. The filtrate was concentrated and purified by silica gel chromatography (0-100% ethyl acetate/hexanes) to yield (5S)-5-[(4-tert-butylphenoxy)methyl]-3-thieno[2,3-c]pyridin-3-yl-1,3-oxazolidin-2-one (EXAMPLE 1) as a yellow solid. ¹H NMR (400 MHz, CD₃OD) δ 9.15 (s, 1H), 8.46 (d, 1H, J=5.6 Hz), 8.05 (s, 1H), 7.88 (d, 1H, J=5.6 Hz), 7.35 (d, 2H, 3-8.8 Hz), 6.95 (d, 2H, 3 =8.8 Hz), 5.17 (m, 1H), 4.36 (m, 2H), 4.29 (m, 1H), 4.17 (m, 1H), 1.29 (s, 9H). LRMS m/z (M+H) 383.4 found, 383.1 required.

The following compounds were prepared by using the corresponding aryl halides in place of 3-bromothieno[2,3-c]pyridine (1-5) as described above.

| Ex. | Structure | Name | M/S |
|---|---|---|---|
| 2 | 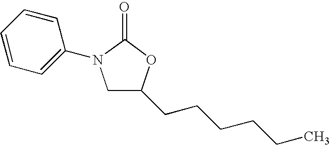 | 5-hexyl-3-phenyl- 1,3-oxazolidin-2-one | LRMS m/z (M + H) 248.1 found, 248.2 required. |
| 3 | 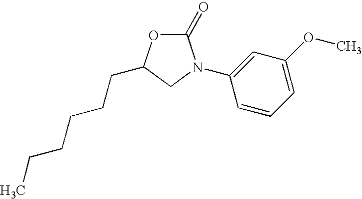 | 5-hexyl-3-(3- methoxyphenyl)-1,3- oxazolidin-2-one | LRMS m/z (M + H) 278.1 found, 278.2 required. |
| 4 | 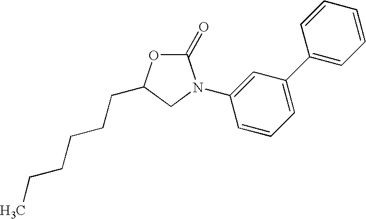 | 3-biphenyl-3-yl-5- hexyl-1,3- oxazolidin-2-one | LRMS m/z (M + H) 324.1 found, 324.2 required. |
| 5 | 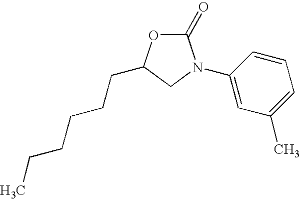 | 5-hexyl-3-(3- methylphenyl)-1,3- oxazolidin-2-one | LRMS m/z (M + H) 262.1 found, 262.2 required. |
| 6 | 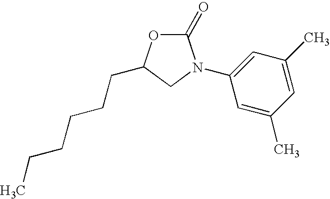 | 3-(3,5- dimethylphenyl)-5- hexyl-1,3- oxazolidin-2-one | LRMS m/z (M + H) 276.2 found, 276.2 required. |
| 7 | 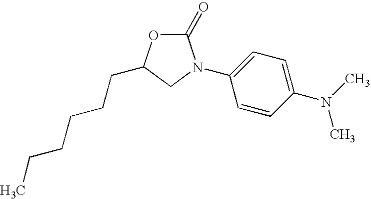 | 3-[4- (dimethylamino)phenyl]- 5-hexyl-1,3- oxazolidin-2-one | LRMS m/z (M + H) 291.2 found, 291.2 required. |
| 8 | 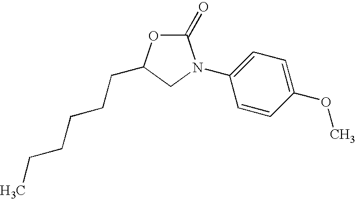 | 5-hexyl-3-(4- methoxyphenyl)-1,3- oxazolidin-2-one | LRMS m/z (M + H) 278.1 found, 278.2 required. |
| 9 | 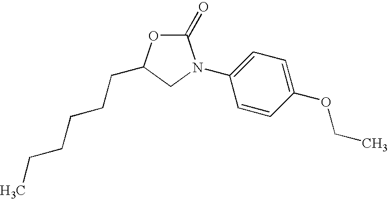 | 3-(4-ethoxyphenyl)- 5-hexyl-1,3- oxazolidin-2-one | LRMS m/z (M + H) 292.2 found, 292.2 required. |
| 10 | 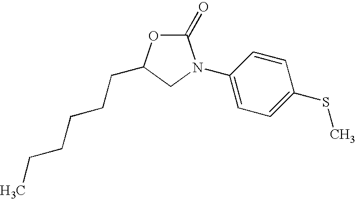 | S-hexyl-3-[4- (methylthio)phenyl]- 1,3-oxazolidin-2-one | LRMS m/z (M + H) 294.1 found, 294.1 required. |
| 11 | 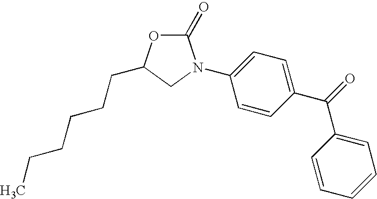 | 3-(4-benzoylphenyl)- 5-hexyl-1,3- oxazolidin-2-one | LRMS m/z (M + H) 352.1 found, 352.2 required. |
| 12 | 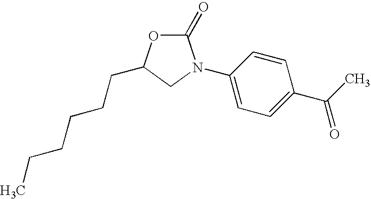 | 3-(4-acetylphenyl)- 5-hexyl-1,3- oxazoIidin-2-one | LRMS m/z (M + H) 290.1 found, 290.2 required. |
| 13 | 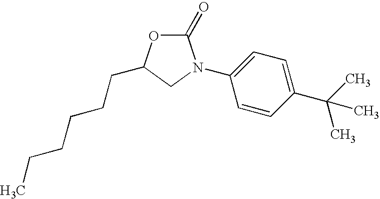 | 3-(4-tert- butylphenyl)-5- hexyl-1,3- oxazolidin-2-one | LRMS m/z (M + H) 304.2 found, 304.2 required. |
| 14 | 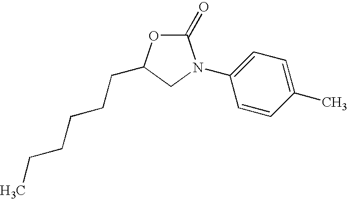 | 5-hexyl-3-(4- methylphenyl)-1,3- oxazolidin-2-one | LRMS m/z (M + H) 262.2 found, 262.2 required. |
| 15 | 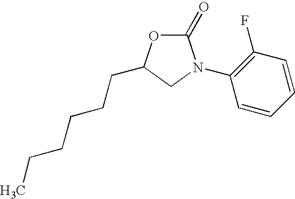 | 3-(2-fluorophenyl)- 5-hexyl-1,3- oxazolidin-2-one | LRMS m/z (M + H) 266.1 found, 266.1 required. |
| 16 | 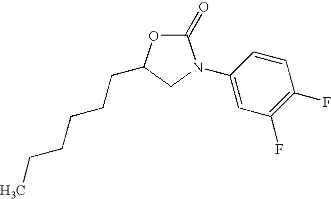 | 3-(3,4- difluorophenyl)-5- hexyl-1,3- oxazolidin-2-one | LRMS m/z (M + H) 284.1 found, 284.1 required. |
| 17 | 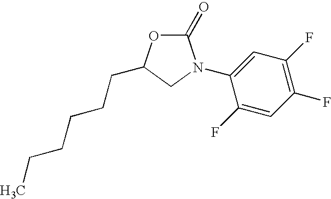 | 5-hexyl-3-(2,4,5- trifluorophenyl)-1,3- oxazolidin-2-one | LRMS m/z (M + H) 302.1 found, 302.1 required. |
| 18 | 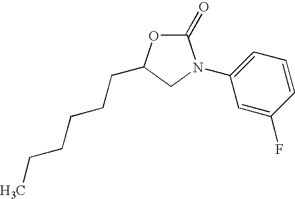 | 3-(3-fluorophenyl)- 5-hexyl-1,3- oxazolidin-2-one | LRMS m/z (M + H) 266.1 found, 266.1 required. |
| 19 | 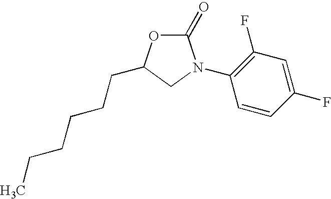 | 3-(2,4- difluorophenyl)-5- hexyl-1,3- oxazolidin-2-one | LRMS m/z (M + H) 284.1 found, 284.1 required. |
| 20 | 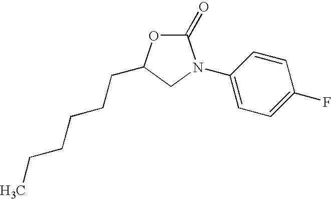 | 3-(4-fluorophenyl)- 5-hexyl-1,3- oxazolidin-2-one | LRMS m/z (M + H) 266.1 found, 266.1 required. |
| 21 | 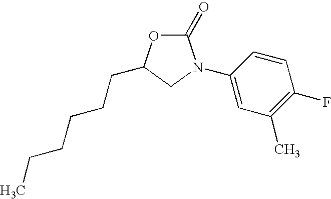 | 3-(4-fluoro-3- methylphenyl)-5- hexyl-1,3- oxazolidin-2-one | LRMS m/z (M + H) 280.2 found, 280.2 required. |
| 22 | 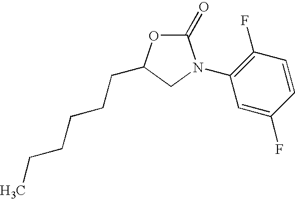 | 3-(2,5- difluorophenyl)-5- hexyl-1,3- oxazolidin-2-one | LRMS m/z (M + H) 284.1 found, 284.1 required. |
| 23 | 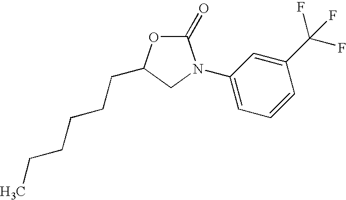 | 5-hexyl-3-[3- (trifluoromethyl) phenyl]-1,3-oxazolidin- 2-one | LRMS m/z (M + H) 316.1 found, 316.1 required. |
| 24 | 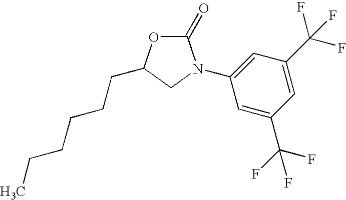 | 3-[3,5- bis(trifluoromethyl) phenyl]-5-hexyl-1,3- oxazolidin-2-one | LRMS m/z (M + H) 384.1 found, 384.1 required. |
| 25 | 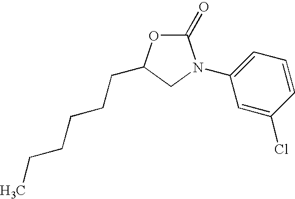 | 3-(3-chlorophenyl)- 5-hexyl-1,3- oxazolidin-2-one | LRMS m/z (M + H) 282.1 found, 282.1 required. |
| 26 | 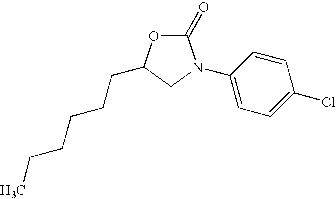 | 3-(4-chlorophenyl)- 5-hexyl-1,3- oxazolidin-2-one | LRMS m/z (M + H) 282.1 found, 282.1 required. |
| 27 | 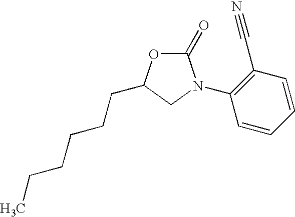 | 2-(5-hexyl-2-oxo-1,3- oxazolidin-3- yl)benzonitrile | LRMS m/z (M + H) 273.2 found, 273.2 required. |
| 28 | 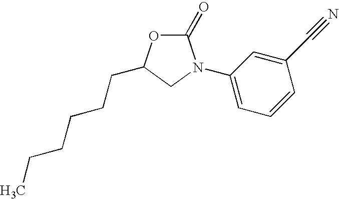 | 3-(5-hexyl-2-oxo-1,3- oxazolidin-3- yl)benzonitrile | LRMS m/z (M + H) 273.1 found, 273.2 required. |
| 29 | 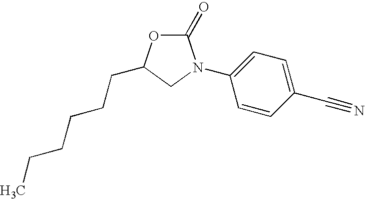 | 4-(5-hexyl-2-oxo-1,3- oxazolidin-3- yl)benzonitrile | LRMS m/z (M + H) 273.2 found, 273.2 required. |
| 30 | 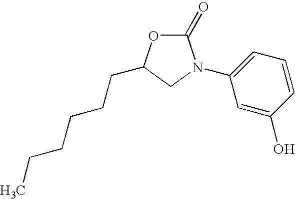 | 5-hexyl-3-(3- hydroxyphenyl)-1,3- oxazolidin-2-one | LRMS m/z (M + H) 264.2 found, 264.2 required. |
| 31 | 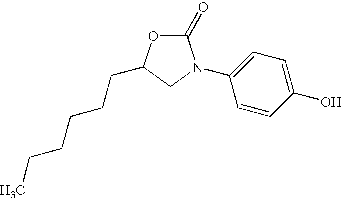 | 5-hexyl-3-(4- hydroxyphenyl)-1,3- oxazolidin-2-one | LRMS m/z (M + H) 264.2 found, 264.2 required. |
| 32 | 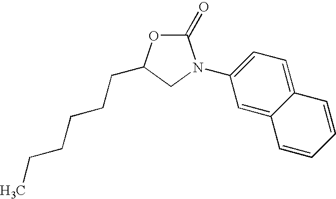 | 5-hexyl-3-(2- naphthyl)-1,3- oxazolidin-2-one | LRMS m/z (M + H) 298.2 found, 298.2 required. |
| 33 | 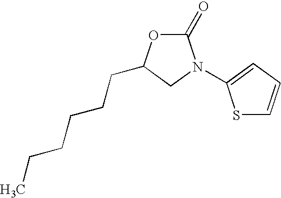 | 5-hexyl-3-(2- thienyl)-1,3- oxazolidin-2-one | LRMS m/z (M + H) 254.1 found, 254.1 required. |
| 34 | 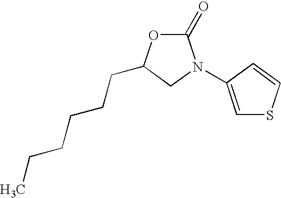 | 5-hexyl-3-(3- thienyl)-1,3- oxazolidin-2-one | LRMS m/z (M + H) 254.1 found, 254.1 required. |
| 35 | 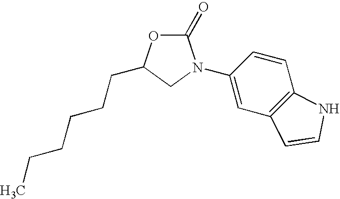 | 5-hexyl-3-(1H-indol- 5-yl)-1,3-oxazolidin- 2-one | LRMS m/z (M + H) 287.2 found, 287.2 required. |
| 36 | 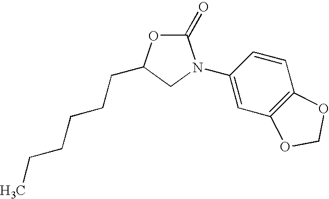 | 3-(1,3-benzodioxol- 5-yl)-5-hexyl-1,3- oxazolidin-2-one | LRMS m/z (M + H) 292.2 found, 292.2 required. |
| 37 | 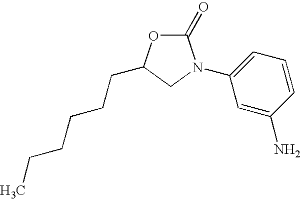 | 3-(3-aminophenyl)- 5-hexyl-1,3- oxazolidin-2-one | LRMS m/z (M + H) 263.2 found, 263.2 required. |
| 38 | 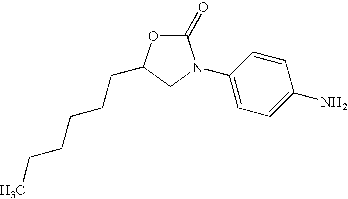 | 3-(4-aminophenyl)- 5-hexyl-1,3- oxazolidin-2-one | LRMS m/z (M + H) 263.2 found, 263.2 required. |
| 39 | 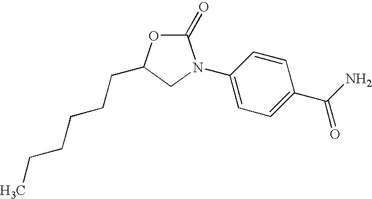 | 4-(5-hexyl-2-oxo-1,3- oxazolidin-3- yl)benzamide | LRMS m/z (M + H) 291.2 found, 291.2 required. |
| 40 | 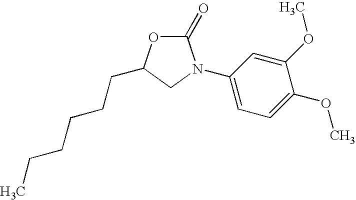 | 3-(3,4- dimethoxyphenyl)- 5-hexyl-1,3- oxazolidin-2-one | LRMS m/z (M + H) 308.2 found, 308.2 required. |
| 41 | 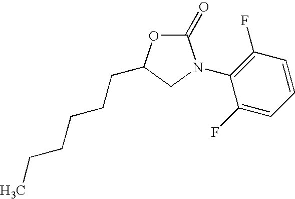 | 3-(2,6- difluorophenyl)-5- hexyl-1,3- oxazolidin-2-one | LRMS m/z (M + H) 284.1 found, 284.1 required. |
| 42 | 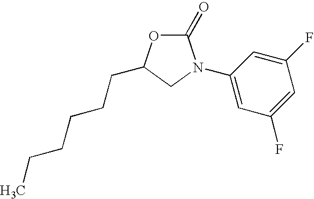 | 3-(3,5- difluorophenyl)-5- hexyl-1,3- oxazolidin-2-one | LRMS m/z (M + H) 284.1 found, 284.1 required. |
| 43 | 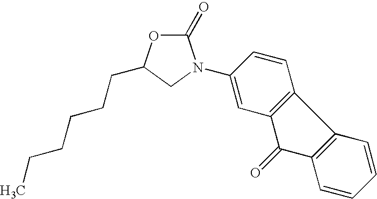 | 5-hexyl-3-(9-oxo- 9H-fluoren-2-yl)- 1,3-oxazolidin-2-one | LRMS m/z (M + H) 350.1 found, 350.2 required. |
| 44 | 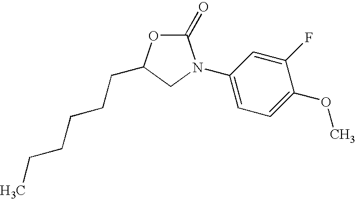 | 3-(3-fluoro-4- methoxyphenyl)-5- hexyl-1,3- oxazolidin-2-one | LRMS m/z (M + H) 296.1 found, 296.2 required. |
| 45 | 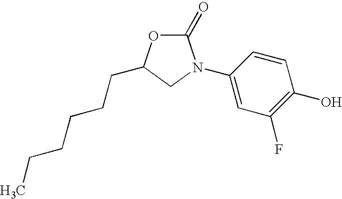 | 3-(3-fluoro-4- hydroxyphenyl)-5- hexyl-1,3- oxazolidin-2-one | LRMS m/z (M + H) 282.1 found, 282.1 required. |
| 46 | 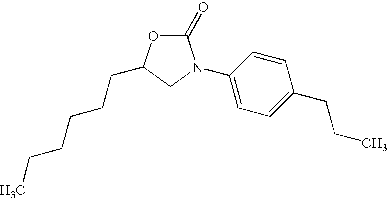 | 5-hexyl-3-(4- propylphenyl)-1,3- oxazolidin-2-one | LRMS m/z (M + H) 290.1 found, 290.2 required. |
| 47 | 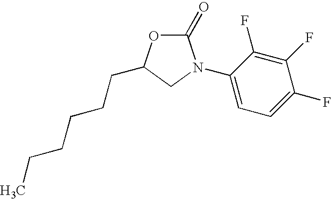 | 5-hexyl-3-(2,3,4- trifluorophenyl)-1,3- oxazolidin-2-one | LRMS m/z (M + H) 302.1 found, 302.1 required. |
| 48 | 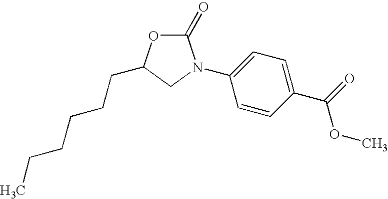 | methyl 4-(5-hexyl-2- oxo-1,3-oxazolidin- 3-yl)benzoate | LRMS m/z (M + H) 306.1 found, 306.2 required. |
| 49 | 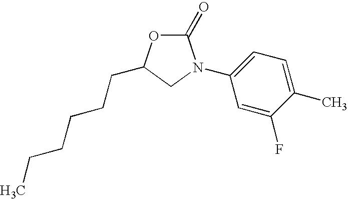 | 3-(3-fluoro-4- methylphenyl)-5- hexyl-1,3- oxazolidin-2-one | LRMS m/z (M + H) 280.1 found, 280.2 required. |
| 50 | 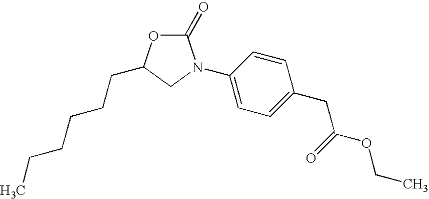 | ethyl [4-(5-hexyl-2- oxo-1,3-oxazolidin- 3-yl)phenyl]acetate | LRMS m/z (M + H) 334.2 found, 334.2 required. |
| 51 | 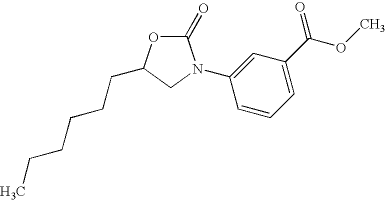 | methyl 3-(5-hexyl-2- oxo-1,3-oxazolidin- 3-yl)benzoate | LRMS m/z (M + H) 306.1 found, 306.2 required. |
| 52 | 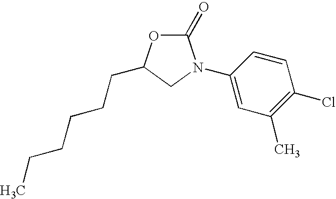 | 3-(4-chloro-3- methylphenyl)-5- hexyl-1,3- oxazolidin-2-one | LRMS m/z (M + H) 296.1 found, 296.1 required. |
| 53 | 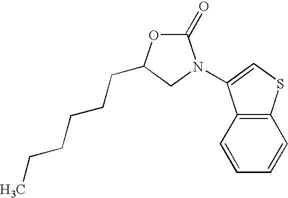 | 3-(1-benzothien-3- yl)-5-hexyl-1,3- oxazolidin-2-one | LRMS m/z (M + H) 304.1 found, 304.1 required. |
| 54 | 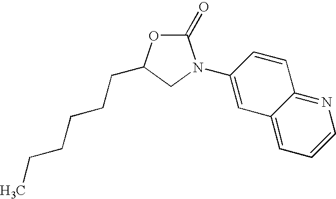 | 5-hexyl-3-quinolin- 6-yl-1,3-oxazolidin- 2-one | LRMS m/z (M + H) 299.1 found, 299.2 required. |
| 55 | 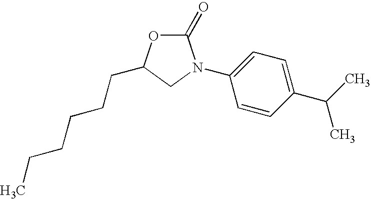 | 5-hexyl-3-(4- isopropylphenyl)- 1,3-oxazolidin-2-one | LRMS m/z (M + H) 290.2 found, 290.2 required. |
| 56 | 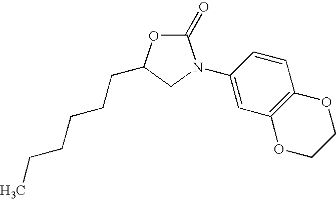 | 3-(2,3-dihydro-1,4- benzodioxin-6-yl)-5- hexyl-1,3- oxazolidin-2-one | LRMS m/z (M + H) 306.1 found, 306.2 required. |
| 57 | 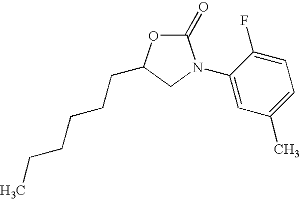 | 3-(2-fluoro-5- methylphenyl)-5- hexyl-1,3- oxazolidin-2-one | LRMS m/z (M + H) 280.1 found, 280.2 required. |
| 58 | 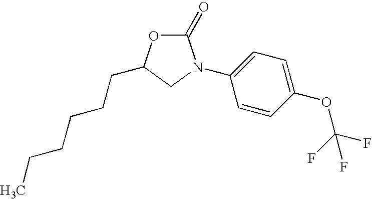 | 5-hexyl-3[4- (trifluoromethoxy) phenyl]-1,3- oxazolidin-2-one | LRMS m/z (M + H) 332.1 found, 332.1 required. |
| 59 | 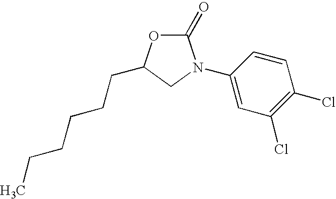 | 3-(3,4- dichlorophenyl)-5- hexyl-1,3- oxazolidin-2-one | LRMS m/z (M + H) 316.0 found, 316.1 required. |
| 60 | 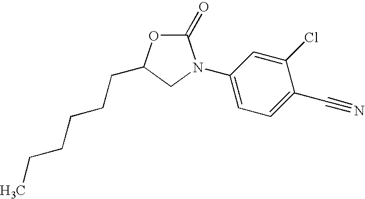 | 2-chloro-4-(5-hexyl- 2-oxo-1,3- oxazolidin-3- yl)benzonitrile | LRMS m/z (M + H) 307.1 found, 307.1 required. |
| 61 | 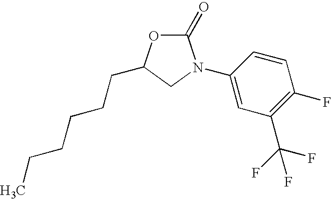 | 3-[4-fluoro-3- (trifluoromethyl) phenyl]-5-hexyl-1,3- oxazolidin-2-one | LRMS m/z (M + H) 334.1 found, 334.1 required. |
| 62 | 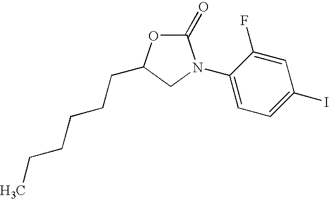 | 3-(2-fluoro-4- iodophenyl)-5-hexyl- 1,3-oxazolidin-2-one | LRMS m/z (M + H) 392.0 found, 392.0 required. |
| 63 | 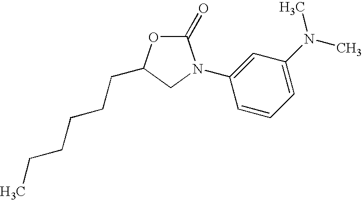 | 3-[3- (dimethylamino) phenyl]-5-hexyl-1,3- oxazolidin-2-one | LRMS m/z (M + H) 291.2 found, 291.2 required. |
| 64 | 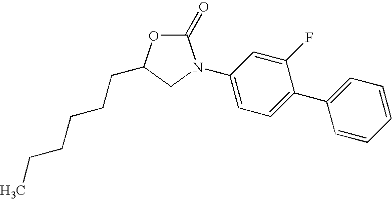 | 3-(2-fluorobiphenyl- 4-yl)-5-hexyl-1,3- oxazolidin-2-one | LRMS m/z (M + H) 342.1 found, 342.2 required. |
| 65 | 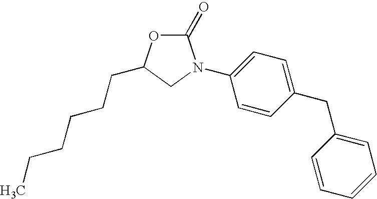 | 3-(4-benzylphenyl)- 5-hexyl-1,3- oxazolidin-2-one | LRMS m/z (M + H) 338.2 found, 338.2 required. |
| 66 | 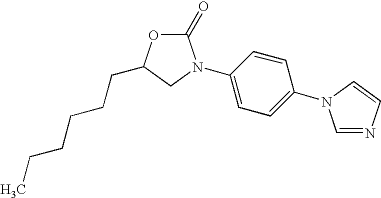 | 5-hexyl-3-[4-(1H- imidazol-1- yl)phenyl]-1,3- oxazolidin-2-one | LRMS m/z (M + H) 314.1 found, 314.2 required. |
| 67 | 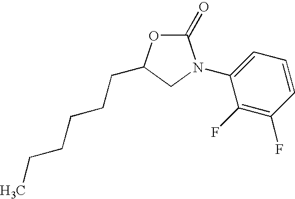 | 3-(2,3- difluorophenyl)-5- hexyl-1,3- oxazolidin-2-one | LRMS m/z (M + H) 284.1 found, 284.1 required. |
| 68 | 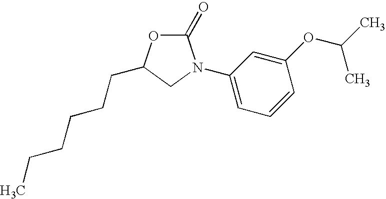 | 5-hexyl-3-(3- isopropoxyphenyl)- 1,3-oxazolidin-2-one | LRMS m/z (M + H) 306.2 found, 306.2 required. |
| 69 | 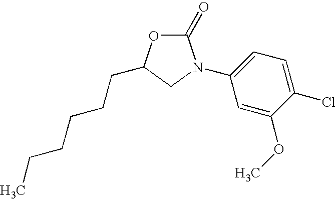 | 3-(4-chloro-3- methoxypheny)-5- hexyl-1,3- oxazolidin-2-one | LRMS m/z (M + H) 312.1 found, 312.1 required. |
| 70 | 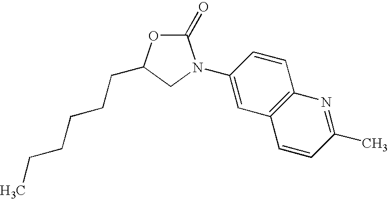 | 5-hexyl-3-(2- methylquinolin-6- yl)-1,3-oxazolidin-2- one | LRMS m/z (M + H) 313.2 found, 313.2 required. |
| 71 | 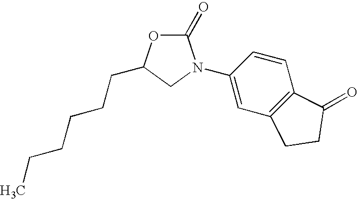 | 5-hexyl-3-(1-oxo-2,3- dihydro-1H-inden- 5-yl)-1,3-oxazolidin- 2-one | LRMS m/z (M + H) 302.2 found, 302.2 required. |
| 72 | 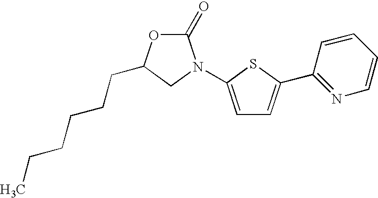 | 5-hexyl-3-(5- pyridin-2-yl-2- thienyl)-1,3- oxazolidin-2-one | LRMS m/z (M + H) 331.1 found, 331.1 required. |
| 73 | 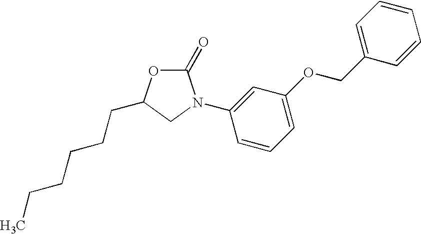 | 3-[3- (benzyloxy)phenyl]- 5-hexyl-1,3- oxazolidin-2-one | LRMS m/z (M + H) 354.2 found, 354.2 required. |
| 74 | 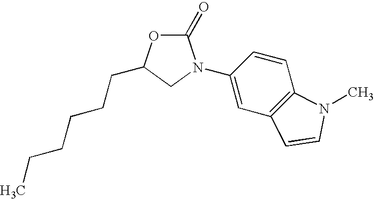 | 5-hexyl-3-(1-methyl- 1H-indol-5-yl)-1,3- oxazolidin-2-one | LRMS m/z (M + H) 301.2 found, 301.2 required. |
| 75 | 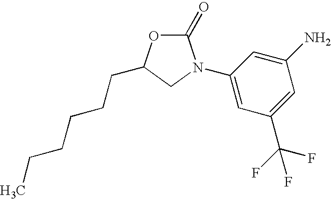 | 3-[3-amino-5- (trifluoromethyl) phenyl]-5-hexyl-1,3- oxazolidin-2-one | LRMS m/z (M + H) 331.2 found, 331.2 required. |
| 76 | 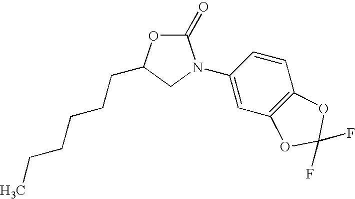 | 3-(2,2-difluoro-1,3- benzodioxo1-5-yl)-5- hexyl-1,3- oxazolidin-2-one | LRMS m/z (M + H) 328.1 found, 328.1 required. |
| 77 | 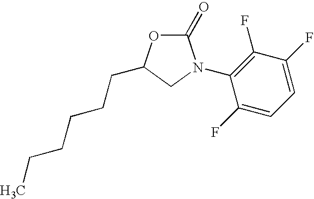 | 5-hexyl-3-(2,3,6- trifluorophenyl)-1,3- oxazolidin-2-one | LRMS m/z (M + H) 302.1 found, 302.1 required. |
| 78 | 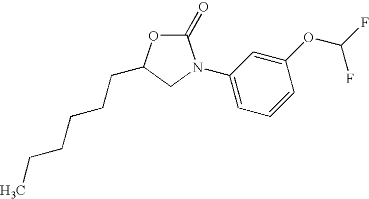 | 3-[3- (difluoromethoxy) phexyl]-5-hexyl-1,3- oxazolidin-2-one | LRMS m/z (M + H) 314.1 found, 314.1 required. |
| 79 | 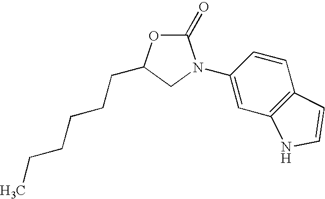 | 5-hexyl-3-(1H-indol- 6-yl)-1,3-oxazolidin- 2-one | LRMS m/z (M + H) 287.1 found, 387.2 required. |
| 80 | 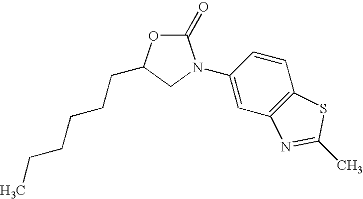 | 5-hexyl-3-(2-methyl- 1,3-benzothiazol-5- yl)-1,3-oxazolidin-2- one | LRMS m/z (M + H) 319.1 found, 319.1 required. |
| 81 | 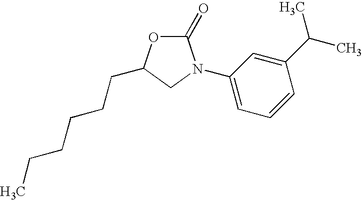 | 5-hexyl-3-(3- isopropylphenyl)- 1,3-oxazolidin-2-one | LRMS m/z (M + H) 290.2 found, 290.2 required. |
| 82 | 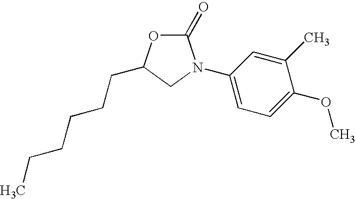 | 5-hexyl-3-(4- methoxy-3- methylphenyl)-1,3- oxazolidin-2-one | LRMS m/z (M + H) 292.2 found, 292.2 required. |
| 83 | 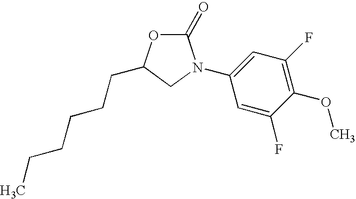 | 3-(3,5-difluoro-4- methoxyphenyl)-5- hexyl-1,3- oxazolidin-2-one | LRMS m/z (M + H) 314.1 found, 314.1 required. |
| 84 | 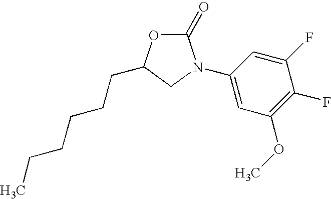 | 3-(3,4-difluoro-5- methoxyphenyl)-5- hexyl-1,3- oxazolidin-2-one | LRMS m/z (M + H) 314.1 found, 314.1 required. |
| 85 | 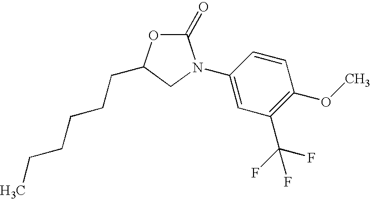 | 5-hexyl-3.-4- methoxy-3- (trifluoromethyl) phenyl]-1,3-oxazolidine 2-one | LRMS m/z (M + H) 346.1 found, 346.2 required. |
| 86 | 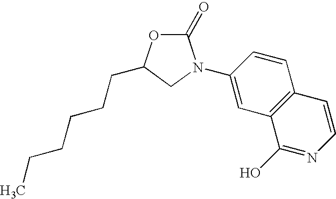 | 5-hexyl-3-(1- hydroxyisoquinolin- 7-yl)-1,3-oxazolidin- 2-one | LRMS m/z (M + H) 315.1 found, 315.2 required. |
| 87 | 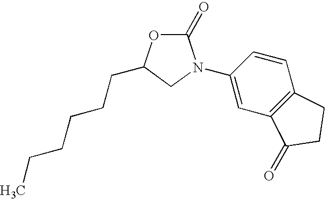 | 5-hexyl-3-(3-oxo-2,3- dihydro-1H-inden- 5-yl)-1,3-oxazolidin- 2-one | LRMS m/z (M + H) 302.1 found, 302.2 required. |
| 88 | 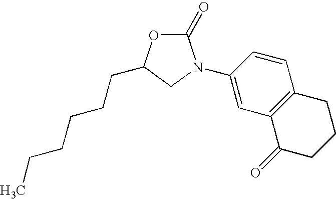 | 5-hexyl-3-(8-oxo- 5,6,7,8- tetrahydronaphthalen- 2-yl)-1,3- oxazolidin-2-one | LRMS m/z (M + H) 316.2 found, 316.2 required. |
| 89 | 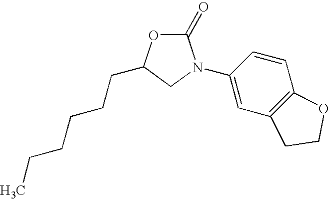 | 3-(2,3-dihydro-1- benzofuran-5-yl)-5- hexyl-1,3- oxazolidin-2-one | LRMS m/z (M + H) 290.1 found, 290.2 required. |
| 90 | 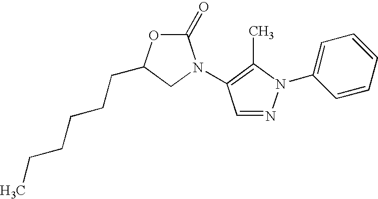 | 5-hexyl-3-(5-methyl- 1-phenyl-1H- pyrazol-4-yl)-1,3- oxazolidin-2-one | LRMS m/z (M + H) 328.2 found, 328.2 required. |
| 91 | 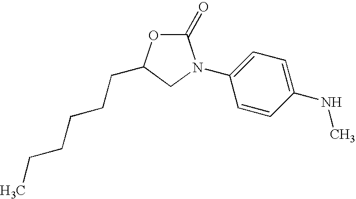 | 5-hexyl-3-[4- (methylamino)phenyl]- 1,3-oxazolidin-2- one | LRMS m/z (M + H) 277.2 found, 277.2 required. |
| 92 | 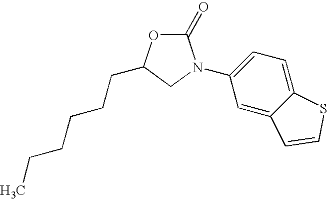 | 3-(1-benzothien-5- yl)-5-hexyl-1,3- oxazolidin-2-one | LRMS m/z (M + H) 304.1 found, 304.1 required. |
| 93 | 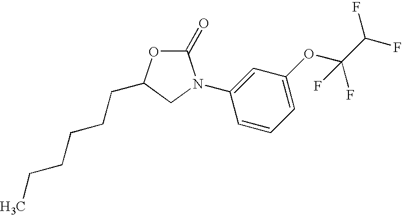 | 5-hexyl-3-[3-(1,1,2,2- tetrafluoroethoxy) phexyl]-1,3- oxazolidin-2-one | LRMS m/z (M + H) 364.1 found, 364.1 required. |
| 94 | 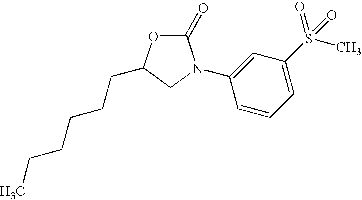 | 5-hexyl-3-[3- (methylsulfonyl) phenyl]-1,3-oxazolidin- 2-one | LRMS m/z (M + H) 326.1 found, 326.1 required. |
| 95 | 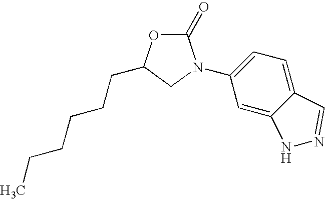 | 5-hexyl-3-(1H- indazo1-6-yl)-1,3- oxazolidin-2-one | LRMS m/z (M + H) 288.1 found, 288.2 required. |
| 96 | 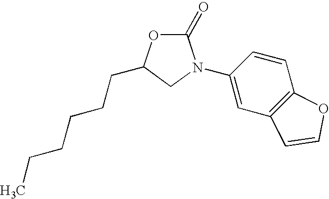 | 3-(1-benzofuran-5- yl)-5-hexyl-1,3- oxazolidin-2-one | LRMS m/z (M + H) 288.1 found, 288.2 required. |
| 97 | 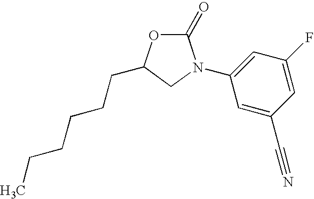 | 3-fluoro-5-(5-hexyl- 2-oxo-1,3- oxazolidin-3- yl)benzonitrile | LRMS m/z (M + H) 291.1 found, 291.1 required. |
| 98 | 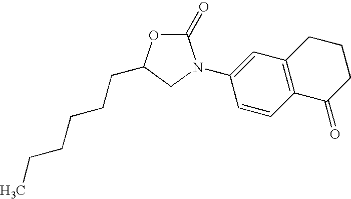 | 5-hexyl-3-(5-oxo- 5,6,7,8- tetrahydronaphthalen- 2-yl)-1,3- oxazolidin-2-one | LRMS m/z (M + H) 316.1 found, 316.2 required. |
| 99 | 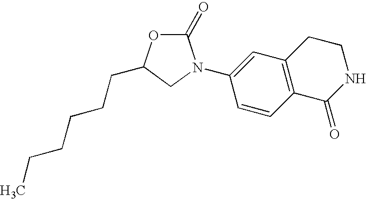 | 6-(5-hexyl-2-oxo-1,3- oxazolidin-3-yl)-3,4- dihydroisoquinolin- 1(2H)-one | LRMS m/z (M + H) 317.1 found, 317.2 required. |
| 100 | 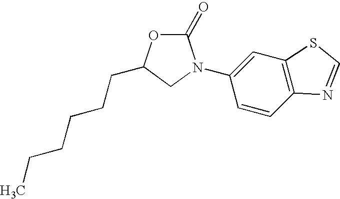 | 3-(1,3-benzothiazol- 6-yl)-5-hexyl-1,3- oxazolidin-2-one | LRMS m/z (M + H) 305.1 found, 305.1 required. |
| 101 | 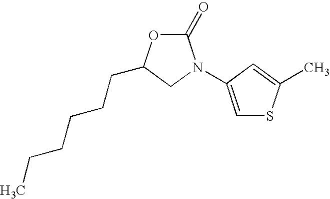 | 5-hexyl-3-(5-methyl- 3-thienyl)-1,3- oxazolidin-2-one | LRMS m/z (M + H) 268.1 found, 268.1 required. |
| 102 | 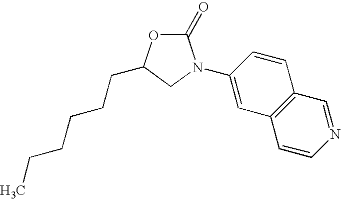 | 5-hexyl-3- isoquinolin-6-yl-1,3- oxazolidin-2-one | LRMS m/z (M + H) 299.1 found, 299.2 required. |
| 103 | 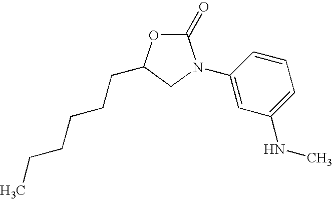 | 5-hexyl-3-[3- (methylamino)phenyl]- 1,3-oxazolidin-2- one | LRMS m/z (M + H) 277.2 found, 277.2 required. |
| 104 | 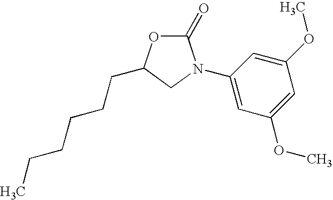 | 3-(3,5- dimethoxyphenyl)- 5-hexyl-1,3- oxazolidin-2-one | LRMS m/z (M + H) 308.1 found, 308.2 required. |
| 105 | 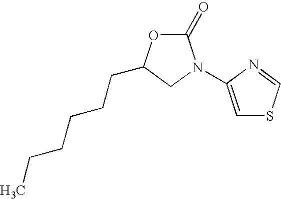 | 5-hexyl-3-(1,3- thiazol-4-yl)-1,3- oxazolidin-2-one | LRMS m/z (M + H) 255.1 found, 255.1 required. |
| 106 | 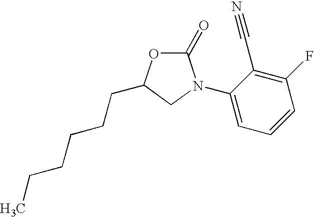 | 2-fluoro-6-(5-hexyl- 2-oxo-1,3- oxazolidin-3- yl)benzonitrile | LRMS m/z (M + H) 291.1 found, 291.1 required. |
| 107 | 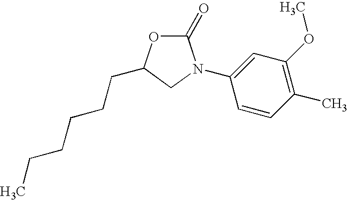 | hexyl-3-(3- methoxy-4- methylphenyl)-1,3- oxazolidin-2-one | LRMS m/z (M + H) 292.1 found, 292.2 required. |
| 108 | 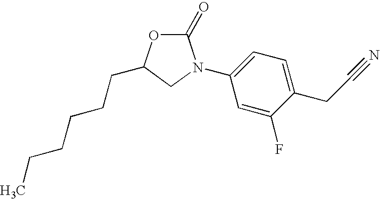 | [2-fluoro-4-(5-hexyl- 2-oxo-1,3- oxazolidin-3- yl)phenyl]acetonitrile | LRMS m/z (M + H) 305.1 found, 305.2 required. |
| 109 | 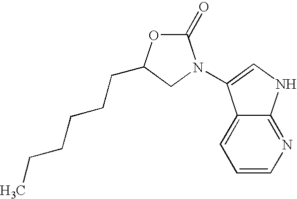 | 5-hexyl-3-(1H- pyrrolo[2,3- b]pyridin-3-yl)-1,3- oxazolidin-2-one | LRMS m/z (M + H) 288.1 found, 288.2 required. |
| 110 | 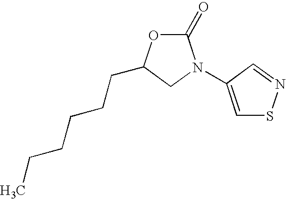 | 5-hexyl-3-isothiazol- 4-yl-1,3-oxazolidin- 2-one | LRMS m/z (M + H) 255.1 found, 255.1 required. |
| 111 | 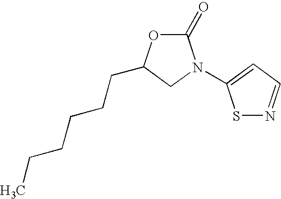 | 5-hexyl-3-isothiazol- 5-yl-1,3-oxazolidin- 2-one | LRMS m/z (M + H) 255.1 found, 255.1 required. |
| 112 | 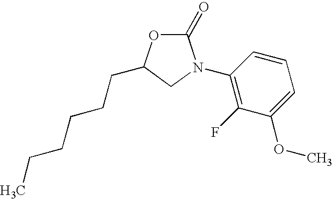 | 3-(2-fluoro-3- methoxyphenyl)-5- hexyl-1,3- oxazolidin-2-one | LRMS m/z (M + H) 296.1 found, 296.2 required. |
| 113 | 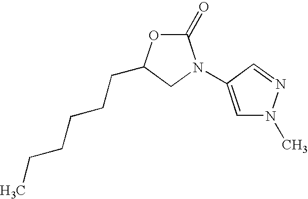 | 5-hexyl-3-(1-methyl- 1H-pyrazol-4-yl)- 1,3-oxazolidin-2-one | LRMS m/z (M + H) 252,1 found, 252.2 required. |
| 114 | 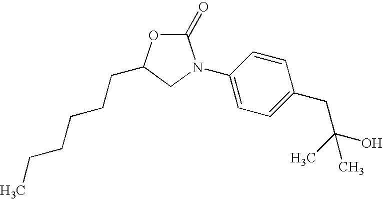 | 5-hexyl-3-[4-(2- hydroxy-2- methylpropyl)phenyl]- 1,3-oxazolidin-2-one | LRMS m/z (M + H) 320.2 found, 320.2 required. |
| 115 | 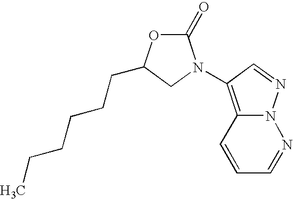 | 5-hexyl-3- pyrazolo[1,5- blpyridazin-3-yl-1,3- oxazolidin-2-one | LRMS m/z (M + H) 289.1 found, 289.2 required. |
| 116 | 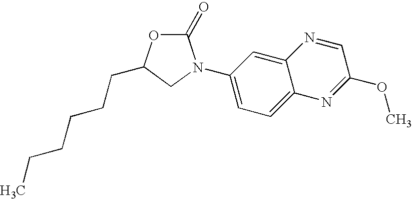 | 5-hexyl-3-(2- methoxyquinoxalin- 6-yl)-1,3-oxazolidin- 2-one | LRMS m/z (M + H) 330.1 found, 330.2 required. |
| 117 | 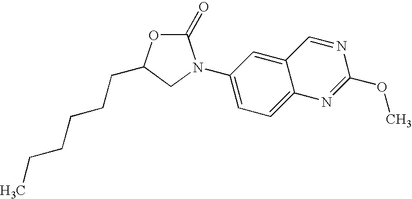 | 5-hexyl-3-(2- methoxyquinazolin- 6-yl)-1,3-oxazolidin- 2-one | LRMS m/z (M + H) 330.1 found, 330.2 required. |
| 118 | 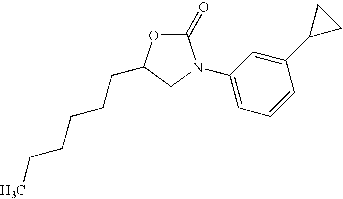 | 3-(3- cyclopropylphenyl)- 5-hexyl-1,3- oxazolidin-2-one | LRMS m/z (M + H) 288.2 found, 288.2 required. |
| 119 | 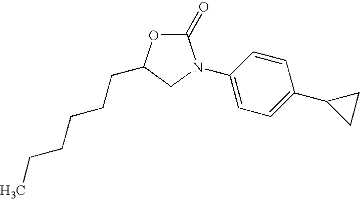 | 3-(4- cyclopropylphenyl)- 5-hexyl-1,3- oxazolidin-2-one | LRMS m/z (M + H) 288.1 found, 288.2 required. |
| 120 | 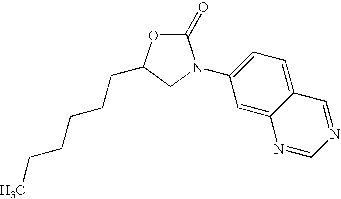 | 5-hexyl-3- quinazolin-7-yl-1,3- oxazolidin-2-one | LRMS m/z (M + H) 300.1 found, 300.2 required. |
| 121 | 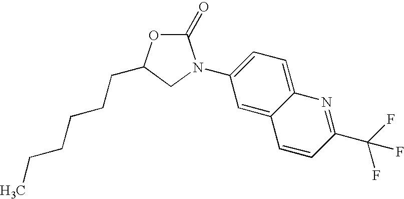 | 5-hexyl-3-[2- (trifluoromethyl) quinolin-6-yl]-1,3- oxazolidin-2-one | LRMS m/z (M + H) 367.1 found, 367.2 required. |
| 122 | 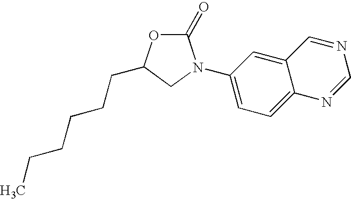 | 5-hexyl-3- quinazolin-6-yl-1,3- oxazolidin-2-one | LRMS m/z (M + H) 300.1 found, 300.2 required. |
| 123 | 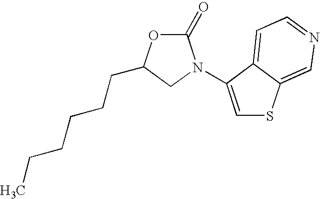 | 5-hexyl-3-thieno[2,3- c]pyridin-3-yl-1,3- oxazolidin-2-one | LRMS m/z (M + H) 305.1 found, 305.1 required. |
| 124 | 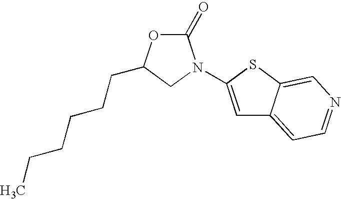 | 5-hexyl-3-thieno[2,3- c]pyridin-2-yl-1,3- oxazolidin-2-one | LRMS m/z (M + H) 305.1 found, 305.1 required. |
| 125 | 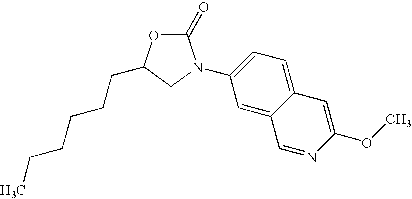 | 5-hexyl-3-(3- methoxylsoquinolin- 7-yl)-1,3-oxazolidin- 2-one | LRMS m/z (M + H) 329.1 found, 329.2 required. |
| 126 | 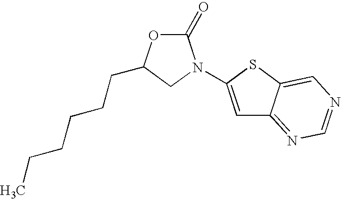 | 5-hexyl-3-thienol[3,2- d]pyrimidin-6-yl- 1,3-oxazolidin-2-one | LRMS m/z (M + H) 306.1 found, 306.1 required. |
| 127 | 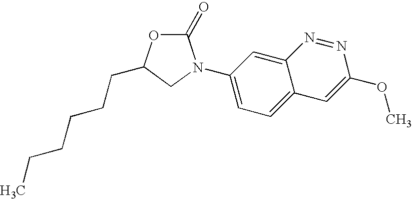 | 5-hexyl-3-(3- methoxycinnolin-7- yl)-4,3-oxazolidin-2- one | LRMS m/z (M + H) 330.1 found, 330.2 required. |
| 128 | 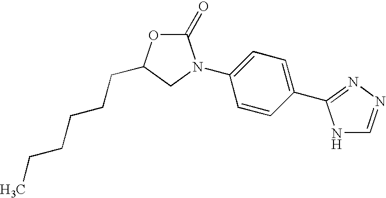 | 5-hexyl-3-[4-(4H- 1,2,4-triazol-3- yl)phenyl]-1,3- oxazolidin-2-one | LRMS m/z (M + H) 315.1 found, 315.2 required. |
| 129 | 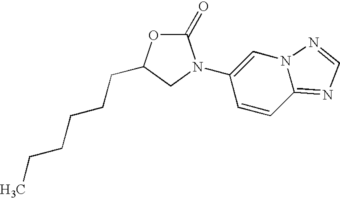 | 5-hexyl-3- [1,2,4]triazolo[1,5- a]pyridin-6-yl-1,3- oxazolidin-2-one | LRMS m/z (M + H) 289.1 found, 289.2 required. |
| 130 | 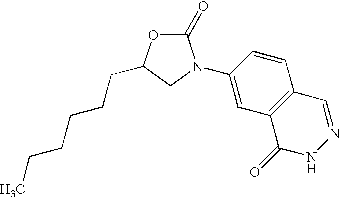 | 7-(5-hexyl-2-oxo-1,3- oxazolidin-3- yl)phthalazin-1(2H)- one | LRMS m/z (M + H) 316.1 found, 316.2 required. |
| 131 | 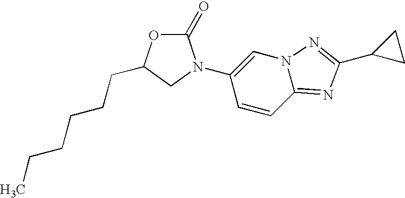 | 3-(2- cyclopropyl[1,2,4] triazolo[1,5-a]pyridin- 6-yl)-5-hexyl-1,3- oxazolidin-2-one | LRMS m/z (M + H) 329.2 found, 329.2 required. |
| 132 | 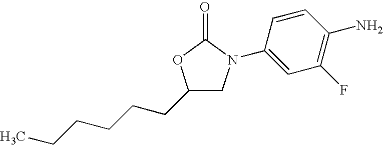 | 3-(4-amino-3- fluorophenyl)-5- hexyl-1,3- oxazolidin-2-one | LRMS m/z (M + H) 281.2 found, 281.2 required. |
| 133 | 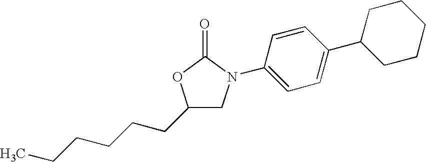 | 3-(4- cyclohexylphenyl)-5- hexyl-1,3- oxazolidin-2-one | LRMS m/z (M + H) 330.3 found, 330.2 required. |

| | | | |
|---|---|---|---|
| 134 | 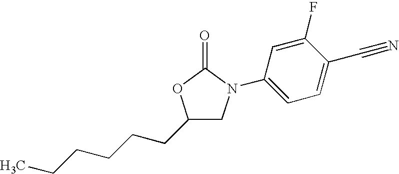 | 2-fluoro-4-(5-hexyl- 2-oxo-1,3- oxazolidin-3- yl)benzonitrile | LRMS m/z (M + H) 291.2 found, 291.1 required. |
| 135 | 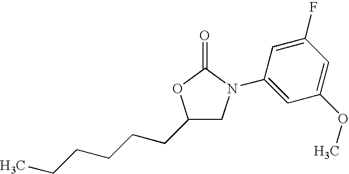 | 3-(3-fluoro-5- methoxyphenyl)-5- hexyl-1,3 - oxazolidin-2-one | LRMS m/z (M + H) 296.2 found, 296.2 required. |
| 136 | 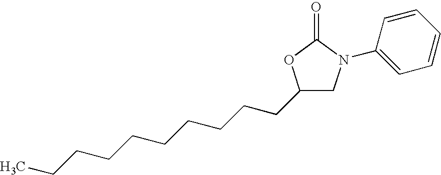 | 5-decyl-3-phenyl- 1,3-oxazolidin-2-one | LRMS m/z (M + H) 304.4 found, 304.2 required. |
| 137 | 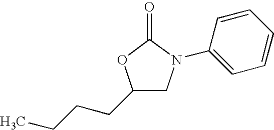 | 5-butyl-3-phenyl- 1,3-oxazolidin-2-one | LRMS m/z (M + H) 220.3 found, 220.1 required. |
| 138 | 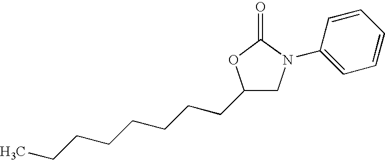 | 5-octyl-3-phenyl-1,3- oxazolidin-2-one | LRMS m/z (M + H) 276.4 found, 276.2 required. |
| 139 | 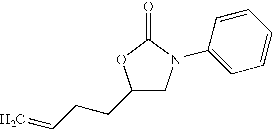 | 5-but-3-en-1-yl-3- phenyl-1,3- oxazolidin-2-one | LRMS m/z (M + H) 218.3 found, 218.1 required. |
| 140 | 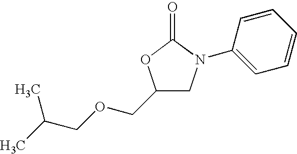 | 5- (isobutoxymethyl)- 3-phenyl-1,3- oxazolidin-2-one | LRMS m/z (M + H) 250.3 found, 250.1 required. |
| 141 | 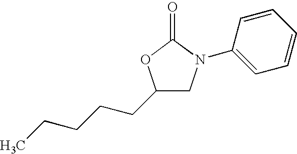 | 5-pentyl-3-phenyl- 1,3-oxazolidin-2-one | LRMS m/z (M + H) 234.3 found, 234.1 required. |
| 142 | 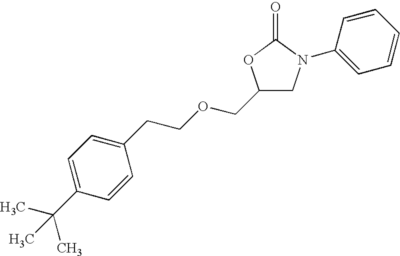 | 5-[(4-tert- butylphenoxy)methyl]- 3-phenyl-1,3- oxazolidin-2-one | LRMS m/z (M + H) 326.4 found, 326.2 required. |
| 143 | 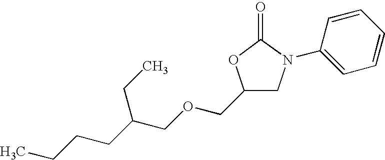 | 5-{[(2- ethylhexyl)oxy]methyl}- 3-phenyl-1,3- oxazolidin-2-one | LRMS m/z (M + H) 306.4 found, 306.2 required. |
| 144 | 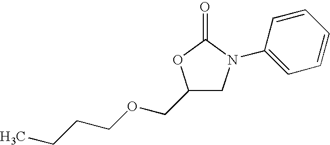 | 5-(butoxymethyl)-3- phenyl-1,3- oxazolidin-2-one | LRMS m/z (M + H) 250.3 found, 250.1 required. |
| 145 | 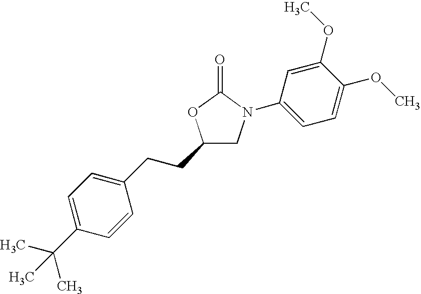 | (5R)-5-[2-(4-tert- butylphenyl)ethyl]- 3-(3,4- dimethoxyphenyl)- 1,3-oxaznlidin-2-one | LRMS m/z (M + H) 384.3 found, 384.2 required. |
| 146 | 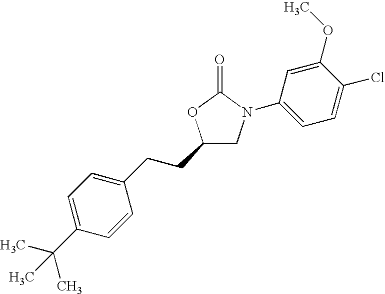 | (5R)-5-[2-(4-tert- butylphenyl)ethyl]- 3-(4-chloro-3- methoxyphenyl)-1,3- oxazolidin-2-one | LRMS m/z (M + H) 388.3 found, 388.2 required. |
| 147 | 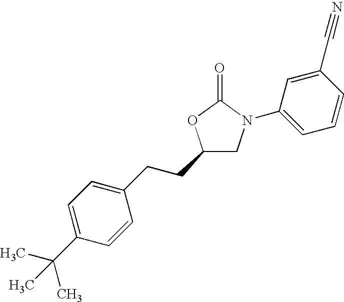 | 3-{(5R)-5-[2-4-tert- butylphenyl)ethyl]- 2-oxo-1,3- oxazolidin-3- yl}benzonitrile | LRMS m/z (M + H) 349.3 found, 349.2 required. |
| 148 | 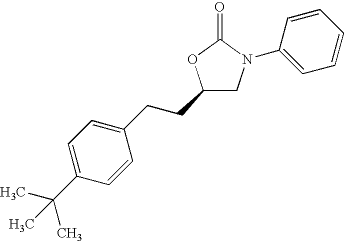 | (5R)-5-[2-(4-tert- butylphenyl)ethyl]- 3-phenyl-1,3- oxazolidin-2-one | LRMS m/z (M + H) 324.3 found, 324.2 required. |
| 149 | 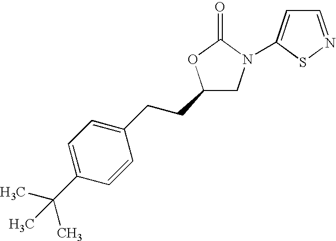 | (5R)-5-(2-(4-tert- butylphenyl)ethyl]- 3-isothiazol-5-yl-1,3- oxazolidin-2-one | LRMS m/z (M + H) 331.3 found, 331.1 required. |
| 150 | 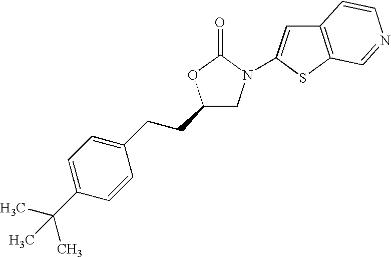 | (5R)-5-[2-(4-tert- butylphenyt)ethyl]- 3-thieno[2,3- c]pyridin-2-yl-1,3- oxazolidin-2-one | LRMS m/z (M + H) 381.3 found, 381.2 required. |
| 151 | 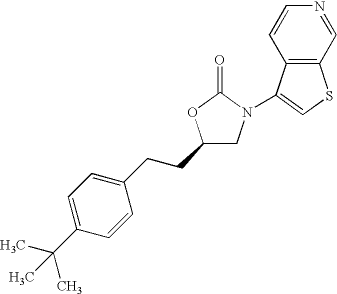 | (5R)-5-[2-(4-tert- butylphenyl)ethyl]- 3-thieno[2,3- c]pyridin-3-yl-1,3- oxazolidin-2-one | LRMS m/z (M + H) 381.4 found, 381.2 required. |
| 152 | 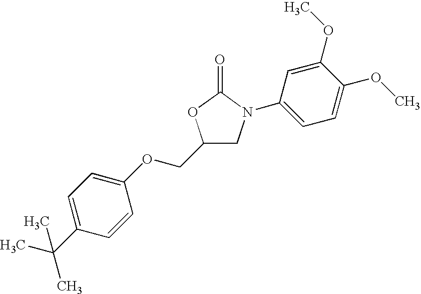 | 5-[(4-tert- butylphenoxy)methyl]- 3-(3,4- dimethoxyphenyl)- 1,3-oxazolidin-2-one | LRMS m/z (M + H) 386.5 found, 386.2 required. |
| 153 | 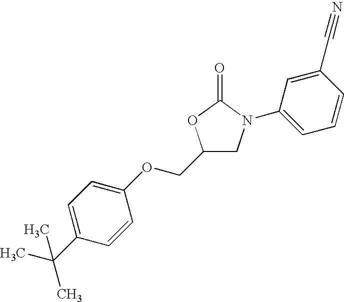 | 3-{5-[(4-tert- butylphenoxy)methyl]- 2-oxo-1,3- oxazolidin-3- yl}benzonitrile | LRMS m/z (M + H) 351.4 found, 351.2 required. |
| 154 | 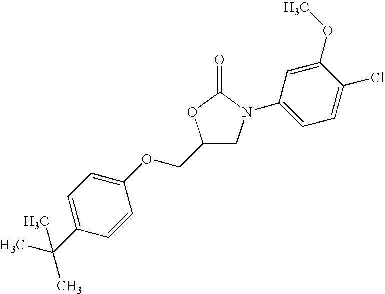 | 5-[(4-tert- butylphenoxy)methyl]- 3-(4-chloro-3- methoxyphenyl)-1,3- oxazolidin-2-one | LRMS m/z (M + H) 390.4 found, 390.1 required. |
| 155 | 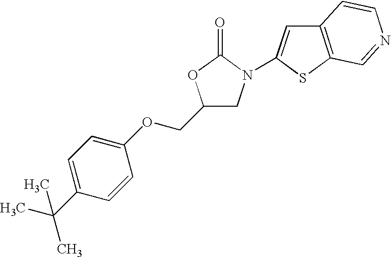 | 5-[(4-tert- butylphenoxy)methyl]- 3-thieno[2,3- c]pyridin-2-yl-1,3- oxazolidin-2-one | LRMS m/z (M + H) 383.5 found, 383.1 required. |
| 156 | 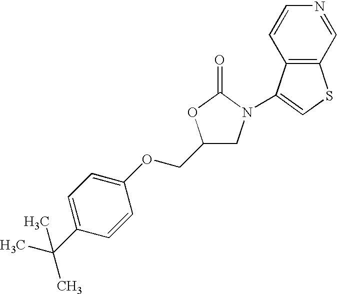 | 5-[(4-tert- butylphenoxy)methyl]- 3-thieno[2,3- c]pyridin-3-yl-1,3- oxazolidin-2-one | LRMS m/z (M + H) 383.5 found, 383.1 required. |
| 157 | 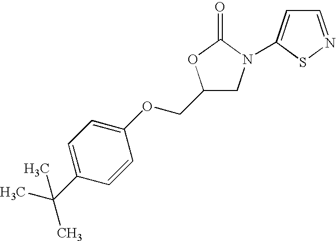 | 5-[(4-tert- butylphenoxy)methyl]- 3-isothiazol-5-yl- 1,3-oxazolidin-2-one | LRMS m/z (M + H) 333.4 found, 333.1 required. |

### Example 158

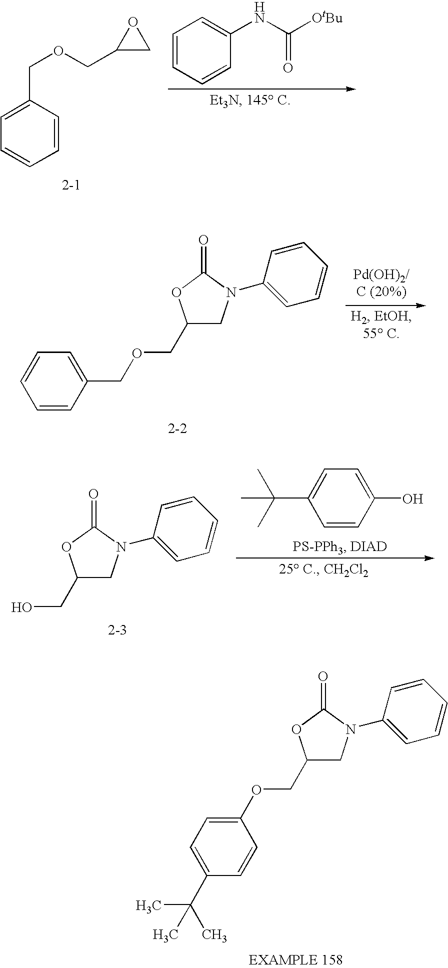

### 5-[(benzyloxy)methyl}-3-phenyl-1,3-oxazolidin-2-one (2-2)

A mixture of N-tert-Boc aniline (28 g, 140 mmol, 1.0 equiv), benzyl glycidyl ether (2-1) (22 mL, 140 mmol, 1.0 equiv) and triethyl amine (1.0 mL, 0.73 g, 7.2 mmol, 0.050 equiv) were heated at 145° C. for 16 hours. The mixture was cooled and the product was purified by silica gel chromatography (0-100% ethyl acetate/hexanes) to yield 5-[(benzyloxy)methyl]-3-phenyl-1,3-oxazolidin-2-one (2-2) as a pale yellow oil. ¹H NMR (400 MHz, CDCl₃) δ 7.55 (m, 2H), 7.37 (m, 7H), 7.15 (m, 1H), 4.79 (m, 1H), 4.61 (d, 2H, J=3.6 Hz), 4.08 (m, 1H), 3.94 (m, 1H), 3.72 (d, 2H, J=4.8 Hz). LRMS m/z (M+H) 284.4 found, 284.1 required.

### 5-(hydroxymethyl)-3-phenyl-1,3-oxazolidin-2-one (2-3)

A mixture of 5-[(benzyloxy)methyl]-3-phenyl-1,3-oxazolidine2-one (2-2) (30 g, 110 mmol, 1.0 equiv) and palladium hydroxide on carbon (20%) (12 g) in ethanol (200 mL) was heated at 55° C. under a hydrogen balloon for 16 hours. The mixture was cooled, filtered through Celite and washed with methanol. The filtrate was concentrated and purified by silica gel chromatography (0-100% ethyl acetate/hexanes) to yield 5-(hydroxymethyl)-3-phenyl-1,3-oxazolidin-2-one (2-3) as a white solid. ¹H NMR (400 MHz, CDCl₃) δ 7.55 (in, 2H), 7.37 (m, 2H), 7.16 (t, 1H, J=7.2 Hz), 4.76 (m, 1H), 4.06 (in, 3H), 3.77 (m, 1H), 2.49 (br s, 1H). LRMS m/z (M+H) 194.2 found, 194.1 required.

### 5-[(4-tert-butylphenoxy)methyl]-3-phenyl-1,3-oxazolidin-2-one (EXAMPLE 158)

To a mixture of 5-(hydroxymethyl)-3-phenyl-1,3-oxazolidin-2-one (2-3) (100 mg, 0.52 mmol, 1.0 equiv), 4-tert-butylphenol (93 mg, 0.62 mmol, 1.2 equiv), and PS-triphenyl phosphine (2 mmol/gram) (0.50 g, 1.0 mmol, 2.0 equiv) in dichloromethane (5 mL) was added DIAD (0.12 mL, 120 mg, 0.62 mmol, 1.2 equiv) and the mixture was stirred at room temp for 16 hours. The mixture was filtered and washed with dichloromethane and methanol. The combined filtrates were concentrated and purified by reverse phase liquid chromatography (Sunfire C18 OBD 5 μm, 20×150 mm column; 0-100% CH₃CN/H₂O gradient w/0.10% TFA present) to yield 5-[(4-Cert-butylphenoxy)methyl]-3-phenyl-1,3-oxazolidin-2-one (EXAMPLE 158) as a white solid. ¹H NMR (400 MHz, CDCl₃) δ 7.58 (d, 2H, J=8.8 Hz), 7.40 (m, 2H), 7.32 (m, 2H), 7.17 (t, 1H, J=7.2 Hz), 6.86 (d, 2H, J=8.8 Hz), 4.98 (m, 1H), 4.22 (m, 3H), 4.07 (m, 1H), 1.29 (s, 9H). LRMS m/z (M+H) 326.4 found, 326.2 required.

The following compounds were prepared by using the corresponding phenols in place of 4-tert-butylphenol as described above.

| Ex. | Structure | Name | M/S |
|---|---|---|---|
| 159 | 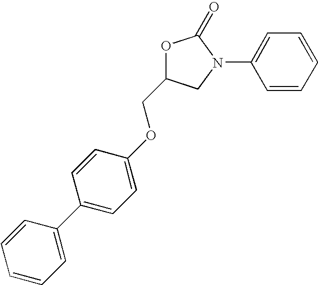 | 5-[(biphenyl-4- yloxy)methyl]-3- phenyl-1,3-oxazolidin- 2-one | LRMS m/z (M + H) 346.1 found, 346.1 required. |
| 160 | 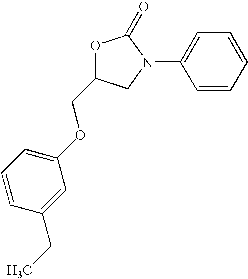 | 5-[(3- ethylphenoxy)methyl]- 3-phenyl-1,3- oxazolidin-2-one | LRMS m/z (M + H) 298.1 found, 298.1 required. |
| 161 | 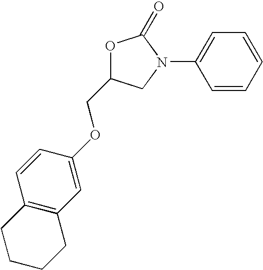 | 3-phenyl-5-[(5,6,7,8- tetrahydronaphthalen- 2-yloxy)methyl]-1,3- oxazolidin-2-one | LRMS m/z (M + H) 324.1 found, 324.2 required. |
| 162 | 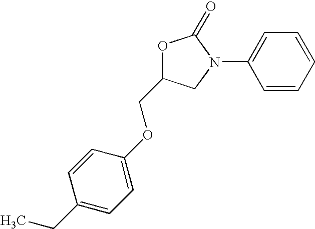 | 5-[(4- ethylphenoxy)methyl]- 3-phenyl-1,3- oxazolidin-2-one | LRMS m/z (M + H) 298.1 found, 298.1 required. |
| 163 | 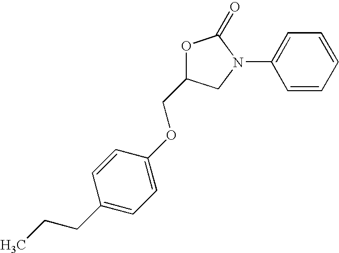 | 3-phenyl-5-[(4- propylphenoxy)methyl]- 1,3-oxazolidin-2-one | LRMS m/z (M + H) 312,1 found, 312.2 required. |
| 164 | 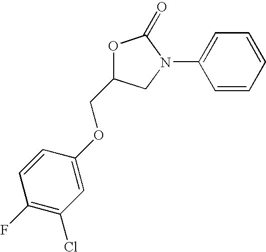 | 5-[(3-chloro-4- fluorophenoxy)methyl]- 3-phenyl-1,3- oxazolidin-2-one | LRMS m/z (M + H) 322.0 found, 322.0 required. |
| 165 | 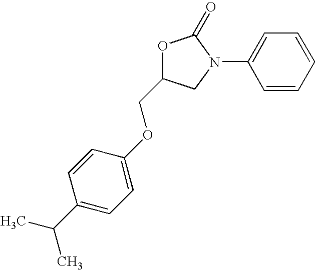 | 5-[(4- isopropylphenoxy)methyl]- 3-phenyl-1,3- oxazolidin-2-one | LRMS m/z (M + H) 312.1 found, 312.2 required. |
| 166 | 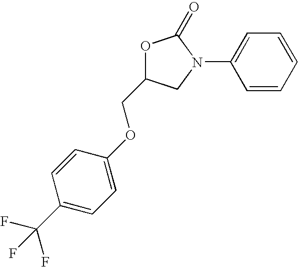 | 3-phenyl-5-{[4- (trifluoromethyl) phenoxylmethyl}-1,3- oxazolidin-2-one | LRMS rn/z (M + H) x 338.0 found, 338.1 required. |
| 167 | 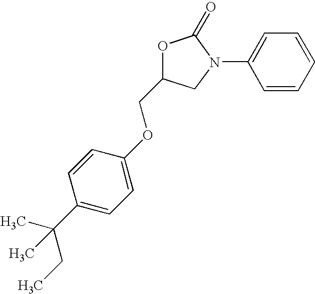 | 5-{[4-(1,1- dimethylpropyl)phenoxy] methyl}-3-phenyl- 1,3-oxazolidin-2-one | LRMS m/z (M + H) 340.1 found, 340.2 required. |
| 168 | 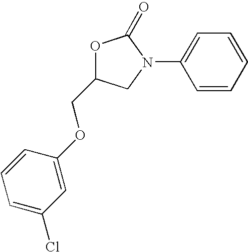 | 5-[(3- chlorophenoxy)methyl]- 3-phenyl-1,3- oxazolidin-2-one | LRMS m/z (M + H) 304.0 found, 304.1 required. |
| 169 | 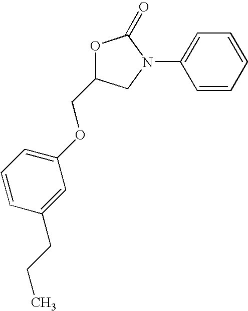 | 3-phenyl-5-[(3- propylphenoxy)methyl]- 1,3-oxazolidin-2-one | LRMS m/z (M + H) 312.1 found, 312.2 required. |
| 170 | 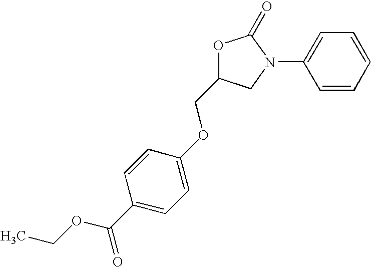 | ethyl 4-[(2-oxo-3- phenyl-1,3-oxazolidin- 5- yl)methoxy]benzoate | LRMS m/z (M + H) 342.1 found, 342.1 required. |
| 171 | 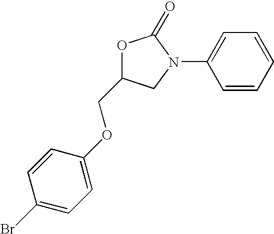 | 5-[(4- bromophenoxy)methyl]- 3-phenyl-1,3- oxazolidin-2-one | LRMS m/z (M + H) 348.0 found, 348.0 required. |
| 172 | 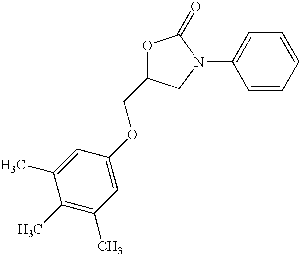 | 3-phenyl-5-[(3,4,5- trimethylphenoxy) methyl]-1,3-oxazolidin-2- one | LRMS m/z (M + H) 312.1 found, 312.2 required. |
| 173 | 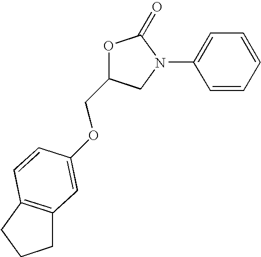 | 5-[(2,3-dihydro-1H- inden-5- yloxy)methyl]-3- phenyl-1,3-oxazolidin- 2-one | LRMS m/z (M + H) 310.1 found, 310.1 required. |
| 174 | 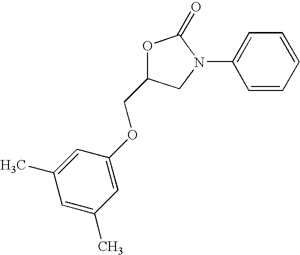 | 5-[(3,5- dimethylphenoxy) methyl]-3-phenyl-1,3- oxazolidin-2-one | LRMS m/z (M + H) 298.1 found, 298.1 required. |
| 175 | 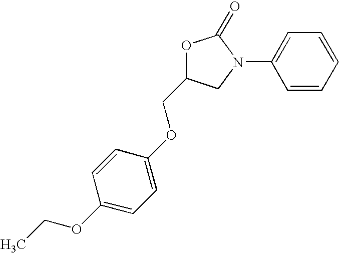 | 5-[(4- ethoxyphenoxy)methyl]- 3-phenyl-1,3- oxazolidin-2-one | LRMS m/z (M + H) 314.1 found, 314.1 required. |
| 176 | 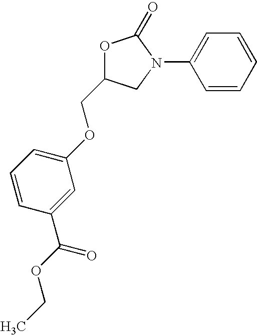 | ethyl 3-[(2-oxo-3- phenyl-1,3-oxazolidin- 5- yl)methoxyl benzoate | LRMS m/z (M + H) 342.1 found, 342.1 required. |
| 177 | 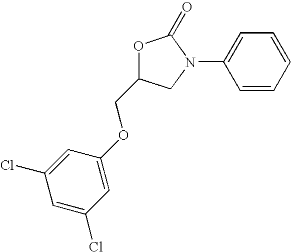 | 5-[(3,5- dichlorophenoxy) methyl]-3-phenyl-1,3- oxazolidin-2-one | LRMS m/z (M + H) 338.0 found, 338.0 required. |
| 178 | 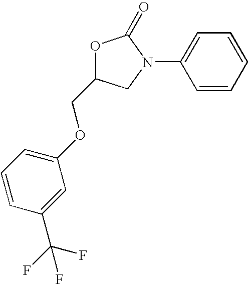 | 3-phenyl-5-{[3- (trifluoromethyl) phenoxy]methyl}-1,3- oxazolidin-2-one | LRMS m/z (M + H) 338.0 found, 338.1 required. |
| 179 | 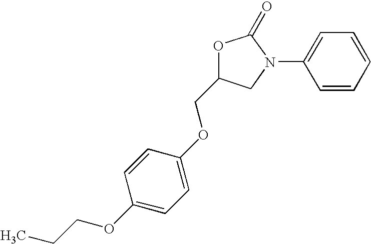 | 3-phenyl-5-[(4- propoxyphenoxy)met hyl]-1,3-oxazolidin-2- one | LRMS m/z (M + H) 328.1 found, 328.1 required. |
| 180 | 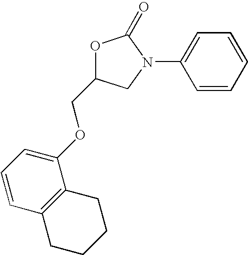 | 3-phenyl-5-[(5,6,7,8- tetrahydronaphthalen- 1-yloxy)methyl]-1,3- oxazolidin-2-one | LRMS m/z (M + H) 324.1 found, 324.1 required. |
| 181 | 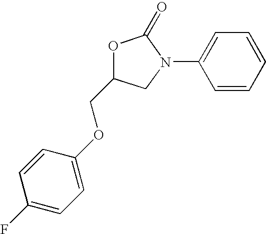 | 5-[(4- fluorophenoxy)methyl]- 3-phenyl-1,3- oxazolidin-2-one | LRMS m/z (M + H) 228.1 found, 228.1 required. |
| 182 | 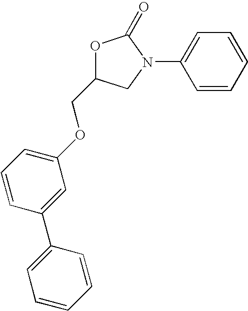 | 5-[(biphenyl-3- yloxy)methyl]-3- phenyl-1,3-oxazolidin- 2-one | LRMS m/z (M + H) 346.1 found, 346.1 required. |
| 183 | 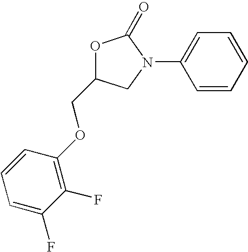 | 5-[(2,3- difluorophenoxy)methyl]- 3-phenyl-1,3- oxazolidin-2-one | LRMS m/z (M + H) 306.0 found, 306.1 required. |
| 184 | 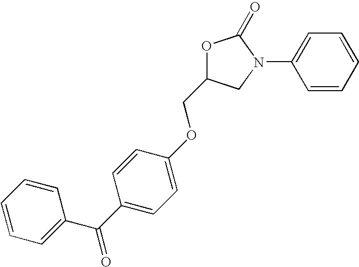 | 5-[(4- benzoylphenoxy)methyl]- 3-phenyl-1,3- oxazolidin-2-one | LRMS m/z (M + H) 374.1 found, 374.1 required. |
| 185 | 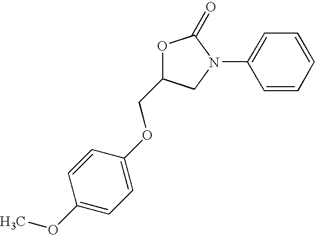 | 5-[(4- methoxyphenoxy)methyl]- 3-phenyl-1,3- oxazolidin-2-one | LRMS m/z (M + H) 300.1 found, 300.1 required. |
| 186 | 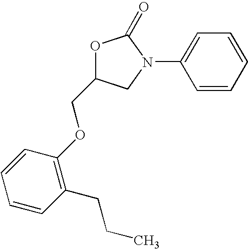 | 3-phenyl-5-[(2- propylphenoxy)methyl]- 1,3-oxazolidin-2-one | LRMS m/z (M + H) 312.1 found, 312.2 required. |
| 187 | 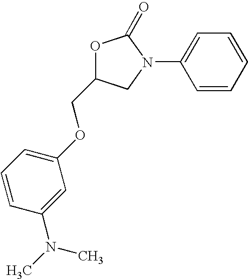 | 5-{[3- (dimethylamino)phenoxy] methyl}-3-phenyl- 1,3-oxazolidin-2-one | LRMS m/z (M + H) 313.1 found, 313.2 required. |
| 188 | 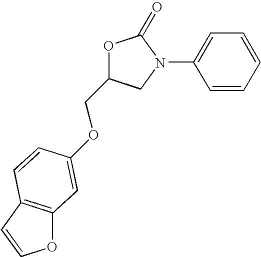 | 5-[(1-benzofuran-6- ytoxy)rnethyl]-3- phenyl-1,3-oxazolidin- 2-one | LRMS m/z (M + H) 310.1 found, 310.1 required. |
| 189 | 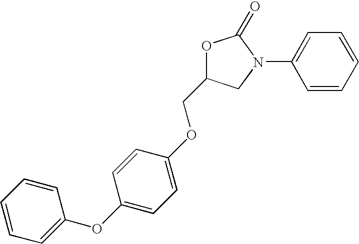 | 5-[(4- phenoxyphenoxy)methyl]- 3-phenyl-1,3- oxazolidin-2-one | LRMS m/z (M + H) 362.1 found, 362.1 required. |
| 190 | 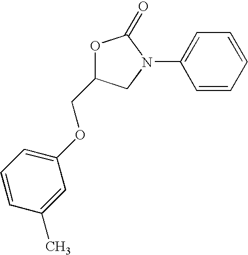 | 5-[(3- methylphenoxy)methyl]- 3-phenyl-1,3- oxazolidin-2-one | LRMS m/z (M + H) 284.1 found, 284.1 required. |
| 191 | 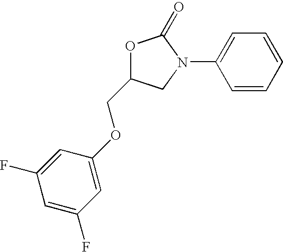 | 5-[(3,5- difluorophenoxy)methyl]- 3-phenyl-1,3- oxazolidin-2-one | LRMS m/z (M + H) 306.0 found, 306.1 required. |
| 192 | 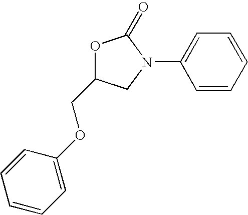 | 5-(phenoxymethyl)-3- phenyl-1,3-oxazolidin- 2-one | LRMS m/z (M + H) 270.1 found, 270.1 required. |
| 193 | 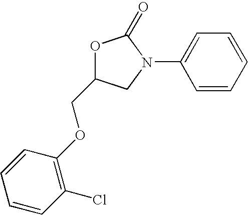 | 5-[(2- chlorophenoxy)methyl]- 3-phenyl-1,3- oxazolidin-2-one | LRMS m/z (M + H) 304.0 found, 304.1 required. |
| 194 | 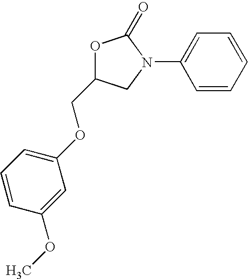 | 5-[(3- methoxyphenoxy) methyl]-3-phenyl-1,3- oxazolidin-2-one | LRMS m/z (M + H) 300.1 found, 300.1 required. |
| 195 | 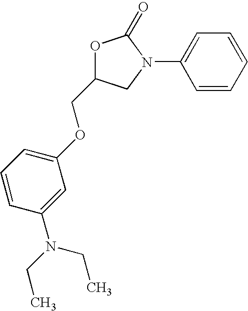 | 5-{[3- (diethylamino)phenoxy] methyl}-3-phenyl- 1,3-oxazolidin-2-one | LRMS m/z (M + H) 341.1 found, 341.2 required. |
| 196 | 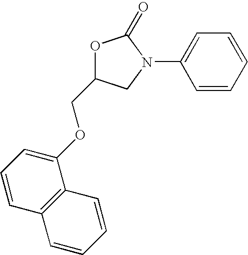 | 5-[(1- naphthyloxy)methyl]- 3-phenyl-1,3- oxazolidin-2-one | LRMS m/z (M + H) 320.1 found, 320.1 required. |
| 197 | 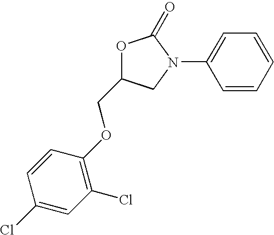 | 5-[(2,4- dichlorophenoxy) methyl]-3-phenyl-1,3- oxazolidin-2-one | LRMS m/z (M + H) 338.0 found, 338.0 required. |
| 198 | 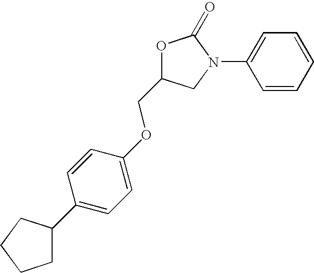 | 5-[(4- cyclopentylphenoxy) methyl]-3-phenyl-1,3- oxazolidin-2-one | LRMS /z (M + H) 338.1 found, 338.2 required. |
| 199 | 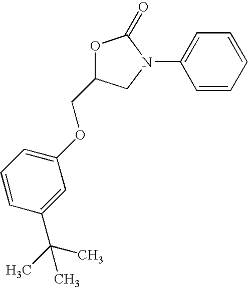 | 5-[(3-tert- butylphenoxy)methyl]- 3-phenyl-1,3- oxazolidin-2-one | LRMS m/z (M + H) 326.1 found, 326.2 required. |
| 200 | 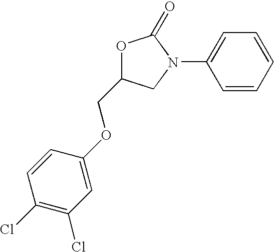 | 5-[(3,4- dichlorophenoxy) methyl]-3-phenyl-1,3- oxazolidin-2-one | LRMS m/z (M + H) 338.0 found, 338.0 required. |
| 201 | 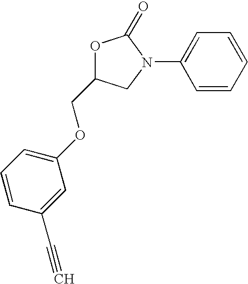 | 5-[(3- ethynylphenoxy)methyl]- 3-phenyl-1,3- oxazolidin-2-one | LRMS m/z (M + H) 294.1 found, 294.1 required. |
| 202 | 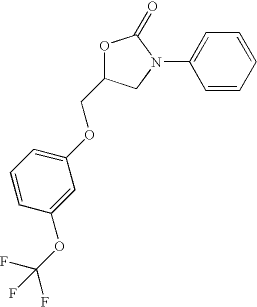 | 3-phenyl-5-{[3- (trifluoromethoxy) phenoxy]methyl}-1,3- oxazolidin-2-one | LRMS m/z (M + H) 354.0 found, 354.1 required. |
| 203 | 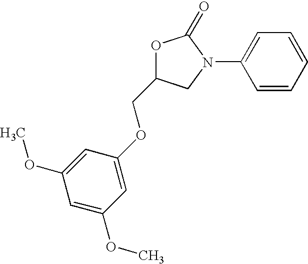 | 5-[(3,5- dimethoxyphenoxy) methyl]-3-phenyl-1,3- oxazolidin-2-one | LRMS m/z (M + H) 330.1 found, 330.1 required. |
| 204 | 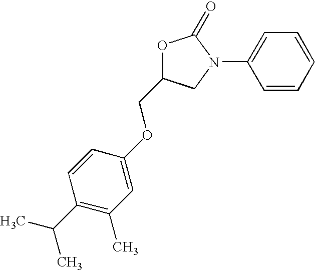 | 5-{(4-isopropyl-3- methylphenoxy)methyl]- 3-phenyl-1,3- oxazolidin-2-one | LRMS m/z (M + H) 326.1 found, 326.2 required. |
| 205 | 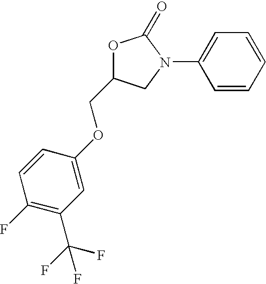 | 5-{[4-fluoro-3- (trifluoromethyl) phenoxy]methyl}-3-phenyl- 1,3-oxazolidin-2-one | LRMS m/z (M + H) 356.0 found, 356.1 required. |
| 206 | 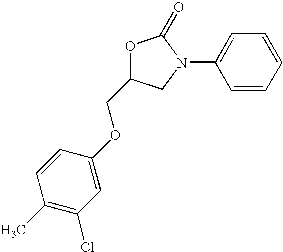 | 5-[(3-chloro-4- methylphenoxy)methyl]- 3-phenyl-1,3- oxazolidin-2-one | LRMS m/z (M + H) 318.0 found, 318.1 required. |
| 207 | 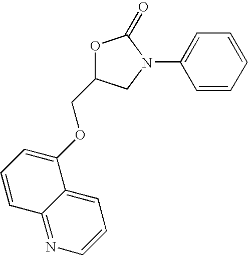 | 3-phenyl-5-[(quinolin- 5-yloxy)methyl]-1,3- oxazolidin-2-one | LRMS m/z (M + H) 321.1 found, 321.1 required. |
| 208 | 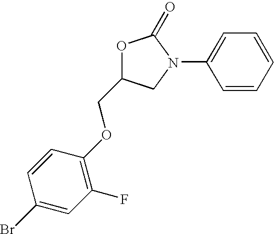 | 5-[(4-bromo-2- fluorophenoxy)methyl]- 3-phenyl-1,3- oxazolidin-2-one | LRMS m/z (M + H) 366.0 found, 366.0 required. |
| 209 | 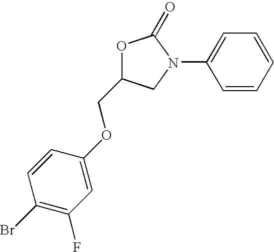 | 5-[(4-bromo-3- fluorophenoxy)methyl]- 3-phenyl-1,3- oxazolidin-2-one | LRMS m/z (M + H) 366.0 found, 366.0 required. |
| 210 | 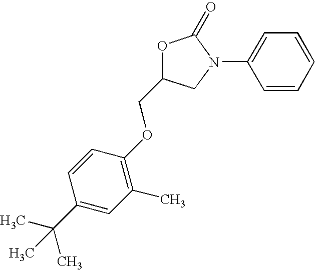 | 5-[(4-tert-butyl-2- methylphenoxy)methyl]- 3-phenyl-1,3- oxazolidin-2-one | LRMS m/z (M + H) 340.1 found, 340.2 required. |
| 211 | 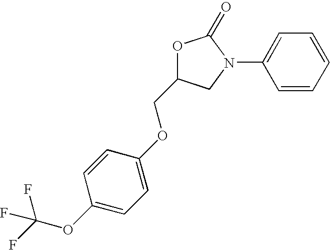 | 3-phenyl-5-{[4- (trifluoromethoxy) phenoxy]methyl}-1,3- oxazolidin-2-one | LRMS m/z (M + H) 354.0 found, 354.1 required. |
| 212 | 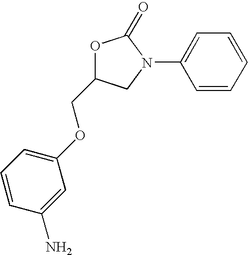 | 5-[(3- aminophenoxy)methyl]- 3-phenyl-1,3- oxazolidin-2-one | LRMS m/z (M + H) 285.1 found, 285.1 required. |
| 213 | 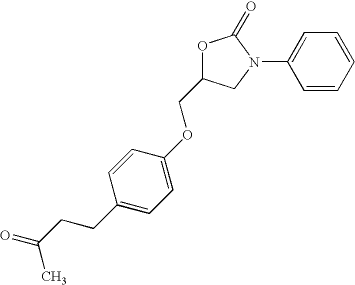 | 5-{[4-(3- oxobutyl)phenoxy] methyl}-3-phenyl-1,3- oxazolidin-2-one | LRMS m/z (M + H) 340.1 found, 340.1 required. |
| 214 | 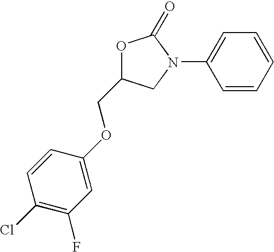 | 5-[(4-chloro-3- fluorophenoxy)methyl]- 3-phenyl-1,3- oxazolidin-2-one | LRMS m/z (M + H) 322.0 found, 322.0 required. |
| 215 | 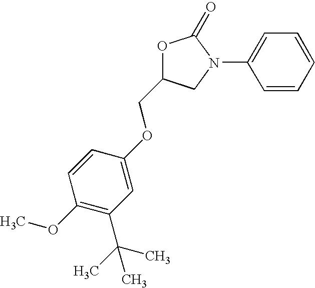 | 5-[(3-tert-butyl-4- methoxyphenoxy) methyl]-3-phenyl-1,3- oxazolidin-2-one | LRMS m/z (M + H) 356.1 found, 356.2 required. |
| 216 | 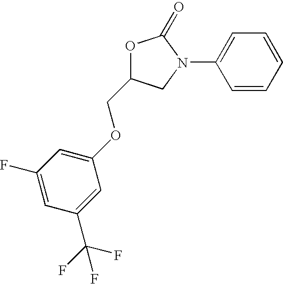 | 5-{[3-fluoro-5- (trifluoromethyl)phenoxy] methyl}-3-phenyl- 1,3-oxazolidin-2-one | LRMS m/z (M + H) 356.0 found, 356.1 required. |
| 217 | 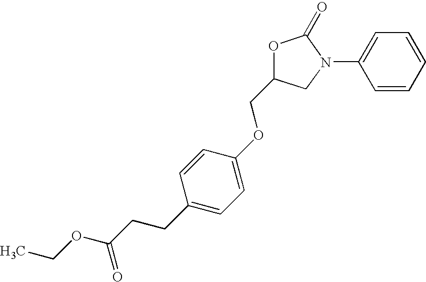 | ethyl 3-{4-[(2-oxo-3- phenyl-1,3-oxazolidin- 5- yl)methoxy]phenyl} propanoate | LRMS m/z (M + H) 370.1 found, 370.2 required. |
| 218 | 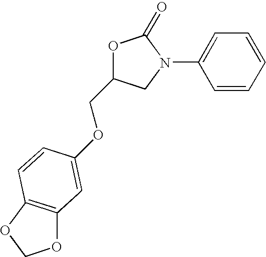 | 5-[(1,3-benzodioxol-5- yloxy)methyl]-3- phenyl-1,3-oxazolidin- 2-one | LRMS m/z (M+ H) 314.0 found, 314.1 required. |
| 219 | 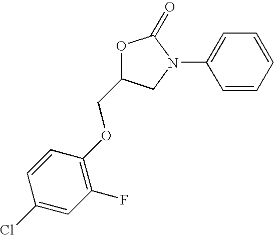 | 5-[(4-chloro-2- fluorophenoxy)methyl]- 3-phenyl-1,3- oxazolidin-2-one | LRMS m/z (M + H) 322.0 found, 322.0 required. |
| 220 | 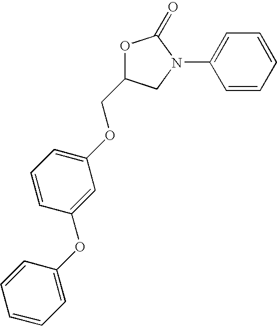 | 5-[(3- phenoxyphenoxy) methyl]-3-phenyl-1,3- oxazolidin-2-one | LRMS m/z (M + H) 362.1 found, 362.1 required. |
| 221 | 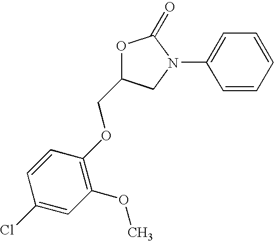 | 5-[(4-chloro-2- methoxyphenoxy) methyl]-3-phenyl-1,3- oxazolidin-2-one | LRMS m/z (M + H) 334.0 found, 334.1 required. |
| 222 | 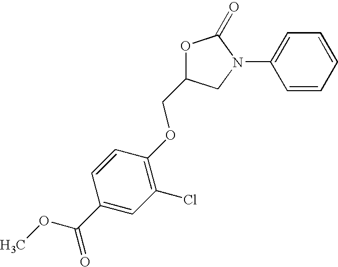 | methyl 3-chloro-4-[(2- oxo-3-phenyl-1,3- oxazolidin-5- yl)methoxy]benzoate | LRMS m/z (M + H) 362.0 found, 362.1 required. |
| 223 | 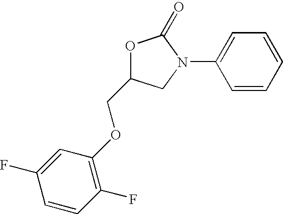 | 5-(2,5- difluorophenoxy) methyl]-3-phenyl-1,3- oxazolidin-2-one | LRMS m/z (M + H) 306.0 found, 306.1 required. |
| 224 | 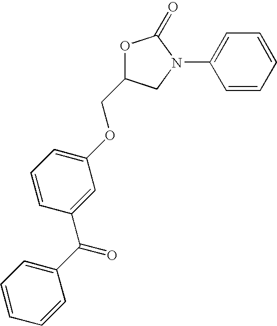 | 5-[(3- benzoylphenoxy)methyl]- 3-phenyl-1,3- oxazolidin-2-one | LRMS m/z (M + H) 374.1 found, 374.1 required. |
| 225 | 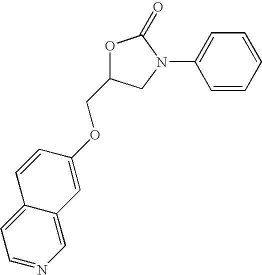 | 5-[(isoquinolin-7- yloxy)methyl]-3- phenyl-1,3-oxazolidin- 2-one | LRMS m/z (M + H) 321.1 found, 321.1 required. |
| 226 | 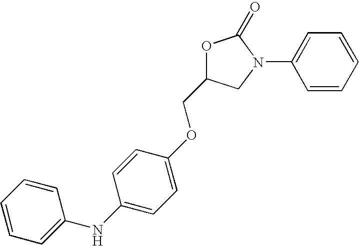 | 5-[(4- anilinophenoxy)methyl]- 3-phenyl-1,3- oxazolidin-2-one | LRMS m/z (M + H) 361.1 found, 361.1 required. |
| 227 | 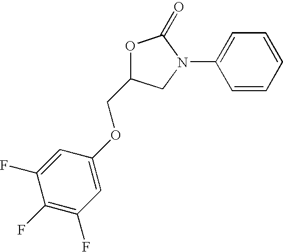 | 3-phenyl-5-[(3,4,5- trifluorophenoxy) methyl]-1,3-oxazolidin-2- one | LRMS m/z (M + H) 324.0 found, 324.1 required. |
| 228 | 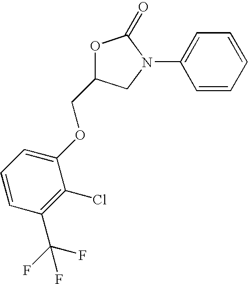 | 5-{[2-chloro-3- (trifluoromethyl)phenoxy] methyl}-3-phenyl- 1,3-oxazolidin-2-one | LRMS m/z (M + H) 372.0 found, 372.0 required. |
| 229 | 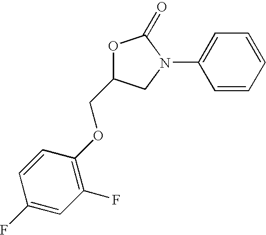 | 5-[(2,4- difluorophenoxy)methyl]- 3-phenyl-1,3- oxazolidin-2-one | LRMS m/z (M + H) 306.0 found, 306.1 required. |
| 230 | 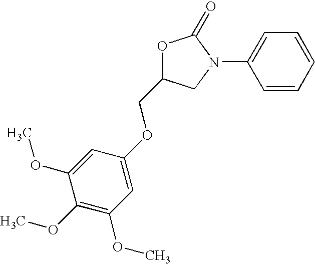 | 3-phenyl-5-[(3,4,5- trimethoxyphenoxy) methyl]-1,3-oxazolidin- 2-one | LRMS m/z (M + H) 360.1 found, 360.1 required. |
| 231 | 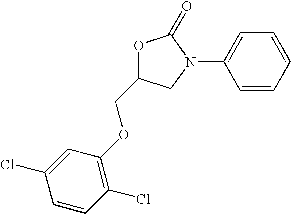 | 5-[(2,5- dichlorophenoxy) methyl]-3-phenyl-1,3- oxazolidin-2-one | LRMS m/z (M + H) 338.0 found, 338.0 required. |
| 232 | 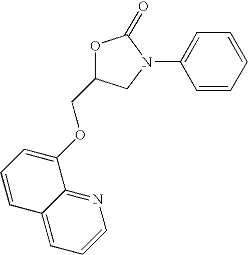 | 3-phenyl-5-[(quinolin- 8-yloxy)methyl]-1,3- oxazolidin-2-one | LRMS m/z (M + H) 321.1 found, 321.1 required. |
| 233 | 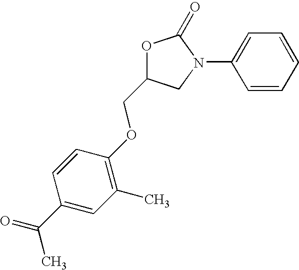 | 5-[(4-acetyl-2- methylphenoxy)methyl]- 3-phenyl-1,3- oxazolidin-2-one | LRMS m/z (M + H) 326.1 found, 326.1 required. |
| 234 | 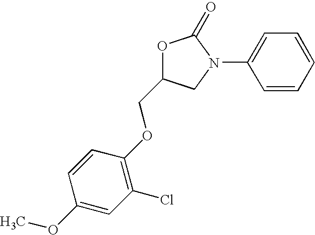 | 5-[(2-chloro-4- methoxyphenoxy) methyl]-3-phenyl-1,3- oxazolidin-2-one | LRMS m/z (M + H) 334.0 found, 334.1 required. |
| 235 | 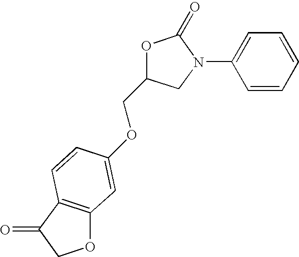 | 5-{[(3-oxo-2,3- dihydro-1- benzofuran-6- yl)oxy]methyl}-3- phenyl-1,3-oxazolidin- | LRMS m/z (M + H) 326.0 found, 326.1 required. |
| 236 | 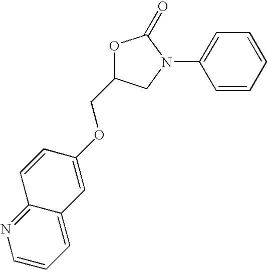 | 3-phenyl-5-[(quinolin- 6-yloxy)methyl]-1,3- oxazolidin-2-one | LRMS m/z (M + H) 321.1 found, 321.1 required. |

### Example 237

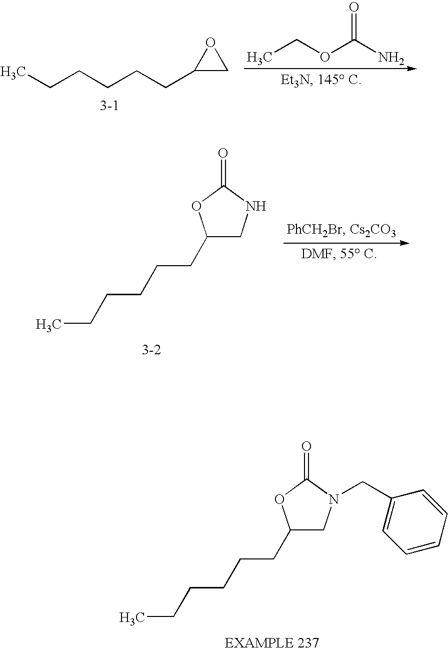

### 5-hexyl-1,3-oxazolidin-2-one (3-2)

A mixture of 1,2-epoxyocate (3-1) (24.1 mL, 20.2 g, 158 mmol, 1.00 equiv), ethyl carbamate (14.0 g, 158 mmol, 1.00 equiv) and triethyl amine (0.210 mL, 0.152 g, 1.51 mmol, 0.010 equiv) was heated at 145° C. for 16 hours. The mixture was cooled, water was added and the precipitate was collected by filtration. The precipitate was purified by silica gel chromatography (0-100% ethyl acetate/hexanes) to yield 5-hexyl-1,3-oxazolidin-2-one (3-2) as a white solid. ¹H NMR (400 MHz, CDCl₃) δ 5.53 (br s, 1H), 4.66 (m, 1H), 3.68 (m, 1H), 3.25 (m, 1H), 1.84 (m, 1H), 1.68 (m, 1H), 1.43 (m, 8H), 0.90 (m, 3H). LRMS m/z (M+H) 172.3 found, 172.1 required.

### 3-benzyl-5-hexyl-1,3-oxazolidin-2-one (EXAMPLE 237)

A mixture of 5-hexyl-1,3-oxazolidin-2-one (3-2) (100 mg, 0.58 mmol, 1.0 equiv), anhydrous cesium carbonate (280 mg, 0.88 mmol, 1.5 equiv) and benzyl bromide (0.069 mL, 0.58 mmol, 1.0 equiv) were heated at 55° C. for 16 hours. The mixture was cooled and filtered. The filtrate was purified by reverse phase liquid chromatography (Sunfire C18 OBD 5 μM, 20×150 mm column; 0-100% CH₃CN/H₂O gradient w/ 0.10% TFA present) to yield 3-benzyl-5-hexyl-1,3-oxazolidin-2-one (EXAMPLE 237) as a white solid. ¹H NMR (400 MHz, CDCl₃) δ 7.38 (m, 5H), 4.49 (m, 3H), 3.48 (m, 1H), 3.03 (in, 1H), 1.73 (m, 1H), 1.58 (m, 1H), 1.31 (m, 8H), 0.89 (m, 3H). LRMS m/z (M+H) 262.4 found, 262.2 required.

The following compounds were prepared by using the appropriate benzyl bromide as described above.

| Ex. | Structure | Name | M/S |
|---|---|---|---|
| 238 | 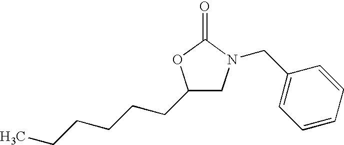 | 3-benzyl-5-hexyl-1,3- oxazolidin-2-one | LRMS m/z (M + H) 262.2 found, 262.2 required. |
| 239 | 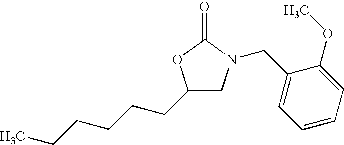 | 5-hexyl-3-(2- methoxybenzyl)-1,3- oxazolidin-2-one | LRMS m/z (M + H) 292.3 found, 292.2 required. |
| 240 | 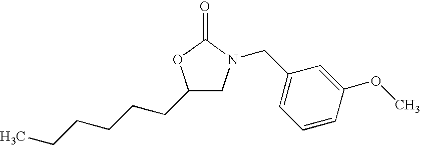 | 5-hexyl-3-(3- methoxybenzyl)-1,3- oxazolidin-2-one | LRMS m/z (M + H) 292.3 found, 292.2 required. |
| 241 | 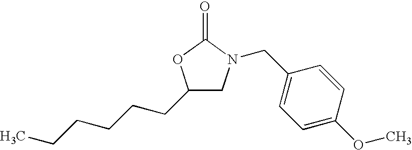 | 5-hexyl-3-(4- methoxybenzyl)-1,3- oxazolidin-2-one | LRMS m/z (M + H) 292.3 found, 292.2 required. |
| 242 | 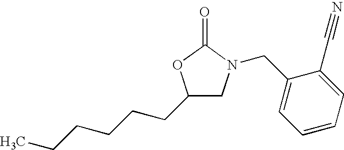 | 2-[(5-hexyl-2-oxo-1,3- oxazolidin-3- yl)methyl]benzonitrile | LRMS m/z (M + H) 287.3 found, 287.2 required. |
| 243 | 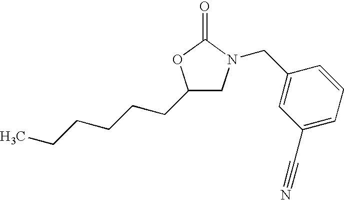 | 3-[(5-hexyl-2-oxo-1,3- oxazolidin-3- yl)methyl]benzonitrile | LRMS m/z (M + H) 287.3 found, 287.2 required. |
| 244 | 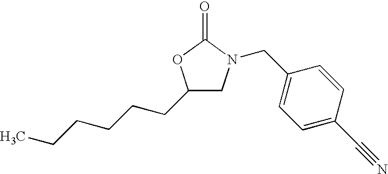 | 4-[(5-hexyl-2-oxo-1,3- oxazolidin-3- yl)methyl]benzonitrile | LRMS m/z (M + H) 287.3 found, 287.2 required. |
| 245 | 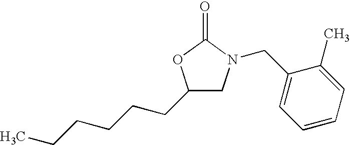 | 5-hexyl-3-(2- methylbenzyl)-1,3- oxazolidin-2-one | LRMS m/z (M + H) 276.3 found, 276.2 required. |
| 246 | 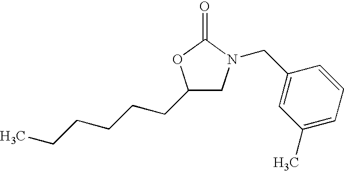 | 5-hexyl-3-(3- methylbenzyl)-1,3- oxazolidin-2-one | LRMS m/z (M + H) 276.3 found, 276.2 required. |
| 247 | 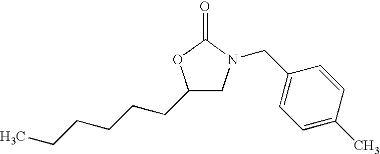 | 5-hexyl-3-(4- methylbenzyl)-1,3- oxazolidin-2-one | LRMS m/z (M + H) 276.3 found, 276.2 required. |
| 248 | 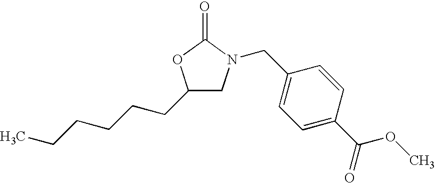 | methyl 4-[(5-hexyl-2- oxo-1,3-oxazolidin-3- yl)methyl]benzoate | LRMS m/z (M + H) 320.4 found, 320.2 required. |
| 249 | 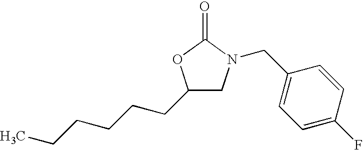 | 3-(4-fluorobenzyl)-5- hexyl-1,3-oxazolidin-2- one | LRMS m/z (M + H) 280.3 found, 280.2 required. |
| 250 | 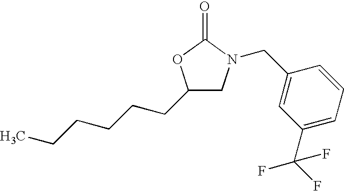 | 5-hexyl-3-[3- (trifluoromethyl)benzyl]- 1,3-oxazolidin-2-one | LRMS m/z (M + H) 330.3 found, 330.2 required. |
| 251 | 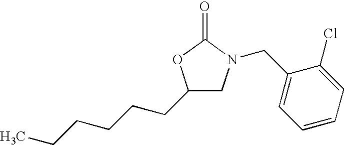 | 3-(2-eblorobenzyl)-5- hexyl-1,3-oxazolidin-2- one | LRMS m/z (M + H) 296.2 found, 296.1 required. |
| 252 | 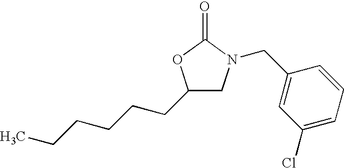 | 3-(3-chlorobenzyl)-5- hexyl-1,3-oxazolidin-2- one | LRMS m/z (M + H) 296.2 found, 296.1 required. |
| 253 | 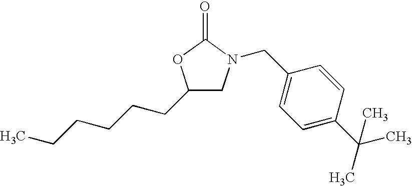 | 3-(4-tent-butylbenzyl)-5- hexyl-1,3-oxazolidin-2- one | LRMS m/z (M + H) 318.4 found, 318.2 required. |
| 254 | 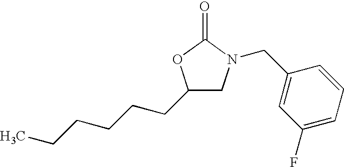 | 3-(3-fluorobenzyl)-5- hexyl-1,3-oxazolidin-2- one | LRMS m/z (M + H) 280.3 found, 280.2 required. |
| 255 | 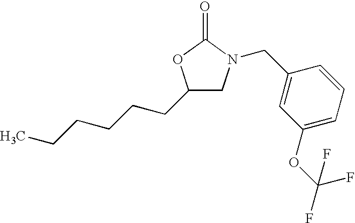 | 5-hexyl-3-[3- (trifluoromethoxy)benzyl]- 1,3-oxazolidin-2-one | LRMS m/z (M + H) 346.3 found, 346.2 required. |
| 256 | 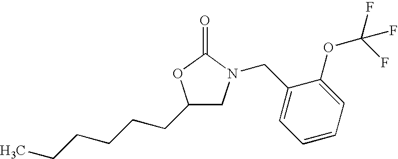 | 5-hexyl-3-[2- (trifluorornetboxy)benzyl]- 1,3-oxazolidin-2-one | LRMS m/z (M + H) 346.3 found, 346.2 required. |
| 257 | 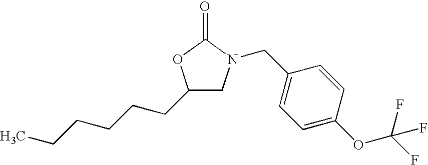 | 5-hexyl-3-[4- (trifluoromethoxy)benzyl]- 1,3-oxazolidin-2-one | LRMS m/z (M + H) 346.3 found, 346.2 required. |
| 258 | 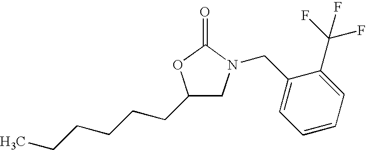 | 5-hexyl-3-[2- (trifluoromethyl)benzyl]- 1,3-oxazolidin-2-one | LRMS m/z (M + H) 330.3 found, 330.2 required. |
| 259 | 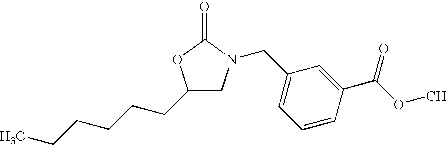 | methyl 3-[(5-hexyl-2- oxo-1,3-oxazolidin-3- yl)methyl]benzoate | LRMS m/z (M + H) 320.4 found, 320.2 required. |
| 260 | 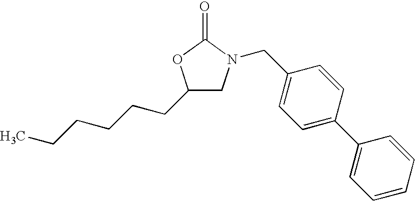 | 3-(biphenyl-4- ylmethyl)-5-hexyl-1,3- oxazolidin-2-one | LRMS m/z (M + H) 338.4 found, 338.2 required. |
| 261 | 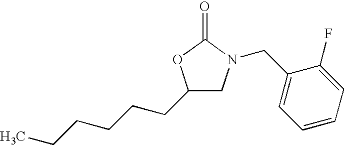 | 3-(2-fluorobenzyl)-5- hexyl-1,3-oxazolidin-2- one | LRMS m/z (M + H) 280.3 found, 280.2 required. |
| 262 | 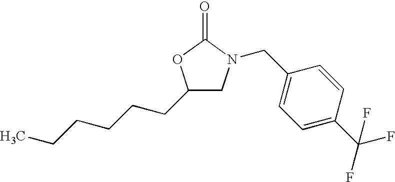 | 5-hexyl-3-[4- (trifluoromethy)benzyl]- 1,3-oxazolidin-2-one | LRMS m/z (M + H) 330.3 found, 330.2 required. |
| 263 | 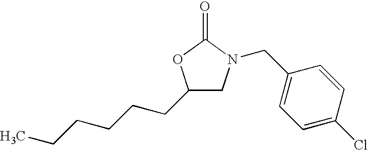 | 3-(4-chlorobenzyl)-5- hexyl-1,3-oxazolidin-2- one | LRMS m/z (M + H) 296.2 found, 296.1 required. |

EXAMPLE 264

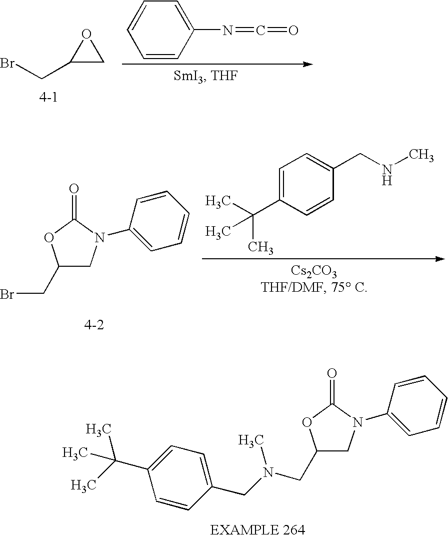

### 5-(bromomethyl)-3-phenyl-1,3-oxazolidin-2-one (4-2)

To a suspension of samarium iodide (3.0 g, 5.6 mmol, 0.08 equiv) in THF (300 mL) under nitrogen was added epibromohydrin (4-1) (6.4 mL, 10.2 g, 75 mmol, 1.0 equiv) followed by phenyl isocyanate (8.2 mL, 8.9 g, 75 mmol, 1.0 equiv) and the mixture was stirred at 23° C. for 4 hours. The mixture was concentrated, and the residue was partitioned between water (200 mL) and diethyl ether (200 mL). The organic layer was separated, washed with brine, dried over sodium sulfate and concentrated. The residue was purified by silica gel chromatography (0-100% ethyl acetate/hexanes) to yield 5-(bromomethyl)-3-phenyl-1,3-oxazolidin-2-one (4-2) as a yellow solid. ¹H NMR (400 MHz, CDCl₃) δ 7.55 (m, 2H), 7.41 (m, 2H), 7.18 (m, 1H), 4.89 (m, 1H), 4.20 (t, 1H, J=8.8 Hz), 3.94 (m, 1H), 3.66 (in, 1H), 3.58 (m, 1H). LRMS m/z (M+H) 256.2 found, 256.0 required.

### 5-{[(4-tert-butylbenzyl)(methyl)amino]methyl}-3-phenyl-1,3-oxazolidin-2-one (EXAMPLE 264)

A mixture of 5-(bromomethyl)-3-phenyl-1,3-oxazolidin-2-one (4-2) (0.10 g, 0.39 mmol, 1 equiv), anhydrous cesium carbonate (0.25 g, 0.77 mmol, 2.0 equiv) and N-methyl-4-tert-butylbenzylamine (0.14 g, 0.78 mmol, 2.0 equiv) in 50/50 THF/DMF (5 mL) was heated at 75° C. for 16 hours. The mixture was filtered and the filtrate was purified by reverse phase liquid chromatography (Sunfire C18 OBD 5 μm, 20×150 mm column; 0-100% CH₃CN/H₂O gradient w/ 0.10% TFA present) to yield 5-{[(4-tert-butylbenzyl)(methyl)amino]methyl}-3-phenyl-1,3-oxazolidin-2-one (EXAMPLE 264). ¹H NMR (400 MHz, CD₃OH) δ 7.56 (m, 4H), 7.49 (d, 2H, J=8.4 Hz), 7.40 (t, 1H, J=8.0 Hz), 7.18 (t, 1H, 7.4 Hz), 5.28 (m, 1H), 4.49 (dd, 2H, J₁=12.8 Hz, J₂=16.8 Hz), 4.29 (t, 1H, J=9.2 Hz), 3.84 (m, 1H), 3.70 (m, 1H), 3.58 (m, 1H), 2.94 (s, 3H), 1.33 (s, 9H). LRMS m/z (M+H) 353.4 found, 353.2 required.

The following compounds were prepared by using the corresponding amines in place of N-methyl-4-tert-butylbenzylamine as described above.

| Ex. | Structure | Name | M/S |
|---|---|---|---|
| 265 | 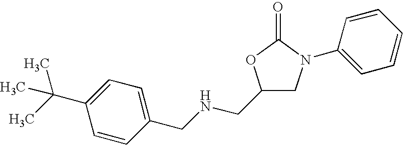 | 5-{[(4-tert- butylbenzyl)amino] methyl}-3-phenyl-1,3- oxazolidin-2-one | LRMS m/z (M + H) 339.3 found, 339.2 required. |
| 266 | 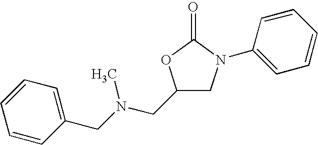 | 5- {[benzyl(methyl)amino] methyl}-3-phenyl- 1,3-oxazolidin-2-one | LRMS m/z (M+ H) 297.3 found, 297.2 required. |

What is claimed is:

1. A compound of Formula I 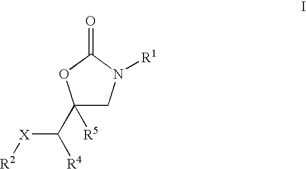
or a pharmaceutically acceptable salt thereof, wherein:
X is selected from the group consisting of: —CH₂—, —O— and —N(R)—;
R¹ is selected from the group consisting of: aryl, —C₁₋₄alkylene-aryl, heteroaryl and —C₁₋₄alkylene-heteroaryl, wherein said aryl, heteroaryl, the aryl portion of —C₁₋₄alkylene-aryl and the heteroaryl portion of —C₁₋₄alkylene-heteroaryl are optionally substituted from one up to the maximum number of substitutable positions with R³;
R² is selected from the group consisting of C₂₋₈alkyl, C₂₋₈alkenyl, C₂₋₈alkynyl, C₃₋₆cycloalkyl, aryl, —C₁₋₄alkylene-aryl, heteroaryl and —C₁₋₄alkylene-heteroaryl, wherein said C₁₋₈alkyl, C₂₋₈alkenyl, C₂₋₈alkynyl and C₃₋₆cycloalkyl are optionally substituted with 1 to 3 hydroxy groups and wherein said aryl, heteroaryl, the aryl portion of —C₁₋₄alkylene-aryl and the heteroaryl portion of —C₁₋₄alkylene-heteroaryl, are optionally substituted from one up to the maximum number of substitutable positions with R³;
each R3 is independently selected from the group consisting of:
(1) halo,
(2) C₁₋₈alkyl, optionally substituted with oxo,
(3) C₂₋₆alkenyl,
(4) C₂₋₆alkynyl,
(5) C₁₋₆haloalkyl,
(6) C₁₋₆hydroxyalkyl,
(7) C₃₋₆cycloalkyl,
(8) C₁₋₆alkoxy,
(9) C₁₋₆haloalkoxy,
(10) —CN,
(11) —C₁₋₄alkylene-CN,
(12) —OH,
(13) —C(O)—O—C₁₋₄alkyl,
(14) —C(O)—C₁₋₄alkyl,
(15) —C₁₋₄alkylene-C(O)—O—C₁₋₄alkyl,
(16) —N(R)₂,
(17) —C(O)—N(R)₂,
(18) —S(O)ₖ—C₁₋₄alkyl, wherein k is 0, 1 or 2,
(19) -aryl,
(20) -heteroaryl,
(21) —C(O)-aryl,
(22) —N(R)-aryl,
(23) benzyl,
(24) benzyloxy,
(25) —CO₂H,
(26) —SH,
(27) —SO₂N(R)R,
(28) —N(R)C(O)N(R)R,
(29) —N(R)C(O)C₁₋₄alkyl and
(30) —N(R)SO₂N(R)R,
or two R3 substituents on adjacent atoms may be joined together with the atoms to which they are attached to form a 5- or 6-membered saturated or partially unsaturated monocyclic ring optionally containing 1 or 2 heteroatoms selected from O, S and N, said ring optionally substituted with oxo or 1 to 3 halo groups, or both, and said ring optionally fused with a benzo group;
R⁴ and R⁵ are independently selected from the group consisting of: H and C₁₋₄alkyl, and
each R is independently selected from the group consisting of: H and C₁₋₄alkyl.

2. The compound according to claim 1 wherein
X is CH₂ and
R² is hexyl.

3. The compound according to claim 1 wherein
R² is phenyl, optionally substituted with one to five substituents R³.

4. A compound of Formula Ia 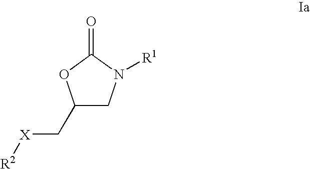
or a pharmaceutically acceptable salt thereof, wherein:
X is selected from the group consisting of: —CH₂— and —O—;
R¹ is selected from the group consisting of aryl, benzyl and heteroaryl, wherein said aryl, heteroaryl, and the phenyl portion of benzyl are optionally substituted from one up to the maximum number of substitutable positions with R³;
R² is selected from the group consisting of: C₂₋₈alkyl, C₂₋₈alkenyl, C₂₋₈alkynyl, C₃₋₆cycloalkyl and 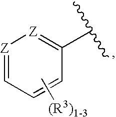
wherein said C₁₋₈alkyl, C₂₋₈alkenyl, C₂₋₈alkynyl and C₃₋₆cycloalkyl are optionally substituted with 1 to 3 hydroxy groups and each Z is independently CH or N;
each R³ is independently selected from the group consisting of:
(1) halo,
(2) C₁₋₈alkyl, optionally substituted with oxo,
(3) C₂₋₆alkenyl,
(4) C₂₋₆alkynyl,
(5) C₁₋₆haloalkyl,
(6) C₁₋₆hydroxyalkyl,
(7) C₃₋₆cycloalkyl,
(8) C₁₋₆alkoxy,
(9) C₁₋₆haloalkoxy,
(10) —CN,
(11) —C₁₋₄alkylene-CN,
(12) —OH,
(13) —C(O)—O—C₁₋₄alkyl,
(14) —C(O)—C₁₋₄alkyl,
(15) —C₁₋₄alkylene-C(O)—O—C₁₋₄alkyl,
(16) —N(R)₂,
(17) —C(O)—N(R)₂,
(18) —S(O)ₖ—C₁₋₄alkyl, wherein k is 0, 1 or 2,
(19) -aryl,
(20) -heteroaryl,
(21) —C(O)-aryl,
(22) —N(R)-aryl,
(23) benzyl,
(24) benzyloxy,
(25) —CO₂H,
(26) —SH,
(27) —SO₂N(R)R,
(28) —N(R)C(O)N(R)R,
(29) —N(R)C(O)C₁₋₄alkyl and
(30) —N(R)SO₂N(R)R,
or two R³ substituents on adjacent atoms may be joined together with the atoms to which they are attached to form a 5- or 6-membered saturated or partially unsaturated monocyclic ring optionally containing 1 or 2 heteroatoms selected from O, S and N, said ring optionally substituted with oxo or 1 to 3 halo groups, or both, and said ring optionally fused with a benzo group; and
each R is independently selected from the group consisting of H and C₁₋₄alkyl.

5. The compound according to claim 4 wherein
X is —CH₂— and
R² is C₂₋₈alkyl, optionally substituted with hydroxy.

6. The compound according to claim 5 wherein R² is n-butyl.

7. The compound according to claim 4 wherein
X is —O— and
R² is

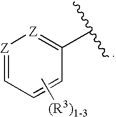

8. The compound according to claim 7 wherein each Z is CH or N.

9. The compound according to claim 8 wherein one R³ is present attached at the para position.

10. The compound according to claim 9 wherein R³ is t-butyl.

11. The compound according to claim 4 wherein R¹ is selected from the group consisting of: phenyl, naphthyl, thienyl, indolyl, benzothienyl, quinolinyl, benzothiazolyl, isoquinolinyl, pyrazolyl, indazolyl, furanyl, benzofuranyl, thiazolyl, pyrrolopyridinyl, isothiazolyl, pyrazolopyradazinyl, quinoxalinyl, quinazolinyl, thienopyridinyl, thienopyrimidinyl, cinnolinyl and triazolopyridinyl, each optionally substituted from one up to the maximum number of substitutable positions with R³.

12. The compound according to claim 4 wherein R¹ is benzyl, wherein the phenyl portion of benzyl is optionally substituted with one to five substituents R³.

13. The compound according to claim 4 wherein R¹ is aryl substituted with two R³ substituents on adjacent atoms that are joined together with the atoms to which they are attached to form a 5- or 6-membered saturated or partially unsaturated monocyclic ring optionally containing 1 or 2 heteroatoms selected from O, S and N, said ring optionally substituted with oxo or 1 to 3 halo groups, or both, and said ring optionally fused with a benzo group.

14. The compound according to claim 13 wherein R¹ is selected from the group consisting of 1,3-benzodioxolyl, 9-oxo-9H-fluorenyl, 2,3-dihydro-1,4-benzodioxinyl, 1-oxo-2,3-dihydro-1H-indenyl, 3-oxo-2,3-dihydro-1H-indenyl, 5-oxo-5,6,7,8-tetrahydronapthalenyl, 2,3-dihydro-1-benzofuranyl, 3,4-dihydroisoquinolin-1(2H)-onyl and phthalazin-1(2H)-onyl.

15. A compound selected from the group consisting of:
(5S)-5-[(4-tert-butylphenoxy)methyl]-3-thieno[2,3-c]pyridin-3-yl-1,3-oxazolidin-2-one;
5-hexyl-3-phenyl-1,3-oxazolidin-2-one;
5-hexyl-3-(3-methoxyphenyl)-1,3-oxazolidin-2-one;
3-biphenyl-3-yl-5-hexyl-1,3-oxazolidin-2-one;
5-hexyl-3-(3-methylphenyl)-1,3-oxazolidin-2-one;
3-(3,5-dimethylphenyl)-5-hexyl-1,3-oxazolidin-2-one;
3-[4-(dimethylamino)phenyl]-5-hexyl-1,3-oxazolidin-2-one;
5-hexyl-3-(4-methoxyphenyl)-1,3-oxazolidin-2-one;
3-(4-ethoxyphenyl)-5-hexyl-1,3-oxazolidin-2-one;
5-hexyl-3-[4-(methylthio)phenyl]-1,3-oxazolidin-2-one;
3-(4-benzoylphenyl)-5-hexyl-1,3-oxazolidin-2-one;
3-(4-acetylphenyl)-5-hexyl-1,3-oxazolidin-2-one;
3-(4-tert-butylphenyl)-5-hexyl-1,3-oxazolidin-2-one;
5-hexyl-3-(4-methylphenyl)-1,3-oxazolidin-2-one;
3-(2-fluorophenyl)-5-hexyl-1,3-oxazolidin-2-one;
3-(3,4-difluorophenyl)-5-hexyl-1,3-oxazolidin-2-one;
5-hexyl-3-(2,4,5-trifluorophenyl)-1,3-oxazolidin-2-one;
3-(3-fluorophenyl)-5-hexyl-1,3-oxazolidin-2-one;
3-(2,4-difluorophenyl)-5-hexyl-1,3-oxazolidin-2-one;
3-(4-fluorophenyl)-5-hexyl-1,3-oxazolidin-2-one;
3-(4-fluoro-3-methylphenyl)-5-hexyl-1,3-oxazolidin-2-one;
3-(2,5-difluorophenyl)-5-hexyl-1,3-oxazolidin-2-one;
5-hexyl-3-[3-(trifluoromethyl)phenyl]-1,3-oxazolidin-2-one;
3-[3,5-bis(trifluoromethyl)phenyl]-5-hexyl-1,3-oxazolidin-2-one;
3-(3-chlorophenyl)-5-hexyl-1,3-oxazolidin-2-one;
3-(4-chlorophenyl)-5-hexyl-1,3-oxazolidin-2-one,
2-(5-hexyl-2-oxo-1,3-oxazolidin-3-yl)benzonitrile;
3-(5-hexyl-2-oxo-1,3-oxazolidin-3-yl)benzonitrile;
4-(5-hexyl-2-oxo-1,3-oxazolidin-3-yl)benzonitrile;
5-hexyl-3-(3-hydroxyphenyl)-1,3-oxazolidin-2-one;
5-hexyl-3-(4-hydroxyphenyl)-1,3-oxazolidin-2-one;
5-hexyl-3-(2-naphthyl)-1,3-oxazolidin-2-one;
5-hexyl-3-(2-thienyl)-1,3-oxazolidin-2-one;
5-hexyl-3-(3-thienyl)-1,3-oxazolidin-2-one;
5-hexyl-3-(1H-indol-5-yl)-1,3-oxazolidin-2-one;
3-(1,3-benzodioxol-5-yl)-5-hexyl-1,3-oxazolidin-2-one;
3-(3-aminophenyl)-5-hexyl-1,3-oxazolidin-2-one;
3-(4-aminophenyl)-5-hexyl-1,3-oxazolidin-2-one;
4-(5-hexyl-2-oxo-1,3-oxazolidin-3-yl)benzamide;
3-(3,4-dimethoxyphenyl)-5-hexyl-1,3-oxazolidin-2-one;
3-(2,6-difluorophenyl)-5-hexyl-1,3-oxazolidin-2-one;
3-(3,5-difluorophenyl)-5-hexyl-1,3-oxazolidin-2-one;
5-hexyl-3-(9-oxo-9H-fluoren-2-yl)-1,3-oxazolidin-2-one;
3-(3-fluoro -4-methoxyphenyl)-5-hexyl-1,3-oxazolidin-2-one;
3-(3-fluoro-4-hydroxyphenyl)-5-hexyl-1,3-oxazolidin-2-one;
5-hexyl-3-(4-propylphenyl)-1,3-oxazolidin-2-one;
5-hexyl-3-(2,3,4-trifluorophenyl)-1,3-oxazolidin-2-one;
methyl 4-(5-hexyl-2-oxo-1,3-oxazolidin-3-yl)benzoate;
3-(3-fluoro-4-methylphenyl)-5-hexyl-1,3-oxazolidin-2-one;
ethyl [4-(5-hexyl-2-oxo-1,3-oxazolidin-3-yl)phenyl]acetate;
methyl 3-(5-hexyl-2-oxo-1,3-oxazolidin-3-yl)benzoate;
3-(4-chloro-3-methylphenyl)-5-hexyl-1,3-oxazolidin-2-one;
3-(1-benzothien-3-yl)-5-hexyl-1,3-oxazolidin-2-one;
5-hexyl-3-quinolin-6-yl-1,3-oxazolidin-2-one;
5-hexyl-3-(4-isopropylphenyl)-1,3-oxazolidin-2-one;
3-(2,3-dihydro-1,4-benzodioxin-6-yl)-5-hexyl-1,3-oxazolidin-2-one;
3-(2-fluoro-5-methylphenyl)-5-hexyl-1,3-oxazolidin-2-one;
5-hexyl-3-[4-(trifluoromethoxy)phenyl]-1,3-oxazolidin-2-one;
3-(3,4-dichlorophenyl)-5-hexyl-1,3-oxazolidin-2-one;
2-chloro-4-(5-hexyl-2-oxo-1,3-oxazolidin-3-yl)benzonitrile;
3-[4-fluoro-3-(trifluoromethyl)phenyl]-5-hexyl-1,3-oxazolidin-2-one;
3-(2-fluoro-4-iodophenyl)-5-hexyl-1,3-oxazolidin-2-one;
3-[3-(dimethylamino)phenyl]-5-hexyl-1,3-oxazolidin-2-one;
3-(2-fluorobiphenyl-4-yl)-5-hexyl-1,3-oxazolidin-2-one;
3-(4-benzylphenyl)-5-hexyl-1,3-oxazolidin-2-one;
5-hexyl-3-[4-(1H-imidazol-1-yl)phenyl]-1,3-oxazolidin-2-one;
3-(2,3-difluorophenyl)-5-hexyl-1,3-oxazolidin-2-one;
5-hexyl-3-(3-isopropoxyphenyl)-1,3-oxazolidin-2-one;
3-(4-chloro-3-methoxyphenyl)-5-hexyl-1,3-oxazolidin-2-one;
5-hexyl-3-(2-methylquinolin-6-yl)-1,3-oxazolidin-2-one;
5-hexyl-3-(1-oxo-2,3-dihydro-1H-inden-5-yl)-1,3-oxazolidin-2-one;
5-hexyl-3-(5-pyridin-2-yl-2-thienyl)-1,3-oxazolidin-2-one;
3[3-(benzyloxy)phenyl]-5-hexyl-1,3-oxazolidin-2-one;
5-hexyl-3-(1-methyl-1H-indol-5-yl)-1,3-oxazolidin-2-one;
3 43-amino-5-(trifluoromethyl)phenyl]-5-hexyl-1,3-oxazolidin-2-one;
3-(2,2-difluoro-1,3-benzodioxol-5-yl)-5-hexyl-1,3-oxazolidin-2-one;
5-hexyl-3-(2,3,6-trifluorophenyl)-1,3-oxazolidin-2-one;
3-[-3-(difluoromethoxy)phenyl]-5-hexyl-1,3-oxazolidin-2-one;
5-hexyl-3-(1H-indol-6-yl)-1,3-oxazolidin-2-one;
5-hexyl-3-(2-methyl-1,3-benzothiazol-5-yl)-1,3-oxazolidin-2-one;
5-hexyl-3-(3-isopropylphenyl)-1,3-oxazolidin-2-one;
5-hexyl-3-(4-methoxy-3-methylphenyl)-1,3-oxazolidin-2-one;
3-(3,5-difluoro-4-methoxyphenyl)-5-hexyl-1,3-oxazolidin-2-one;
3-(3,4-difluoro-5-methoxyphenyl)-5-hexyl-1,3-oxazolidin-2-one;
5-hexyl-3-[4-methoxy-3-(trifluoromethyl)phenyl]-1,3-oxazolidin-2-one;
5-hexyl-3-(1-hydroxyisoquinolin-7-yl)-1,3-oxazolidin-2-one;
5-hexyl-3-(3-oxo-2,3-dihydro-1H-inden-5-yl)-1,3-oxazolidin-2-one;
5-hexyl-3-(8-oxo-5,6,7,8-tetrahydronaphthalen-2-yl)-1,3-oxazolidin-2-one;
3-(2,3-dihydro-1-benzofuran-5-yl)-5-hexyl 1,3-oxazolidin-2-one;
5-hexyl-3-(5-methyl-1-phenyl-1H-pyrazol-4-yl)-1,3-oxazolidin-2-one;
5-hexyl-3 44-(methylamino)phenyl]-1,3-oxazolidin-2-one;
3-(1-benzothien-5-yl)-5-hexyl-1,3-oxazolidin-2-one;
5-hexyl-3-[3-(1,1,2,2-tetrafluoroethoxy)phenyl]-1,3-oxazolidin-2-one;
5-hexyl-3-(methylsulfonyl)phenyl]-1,3-oxazolidin-2-one;
5-hexyl-3-(1H-indazol-6-yl)-1,3-oxazolidin-2-one;
3-(1-benzofuran-5-yl)-5-hexyl-1,3-oxazolidin-2-one;
3-fluoro-5-(5-hexyl-2-oxo -1,3-oxazolidin-3-yl)benzonitrile;
5-hexyl-3-(5-oxo-5,6,7,8-tetrahydronaphthalen-2-yl)-1,3-oxazolidin-2-one;
6-(5-hexyl-2-oxo-1,3-oxazolidin-3-yl)-3,4-dihydroisoquinolin-1 (2H)-one;
3-(1,3-benzothiazol-6-yl)-5-hexyl-1,3-oxazolidin-2-one;
5-hexyl-3-(5-methyl-3-thienyl)-1,3-oxazolidin-2-one;
5-hexyl-3-isoquinolin-6-yl-1,3-oxazolidin-2-one;
5-hexyl-3-[3-(methylamino)phenyl]-1,3-oxazolidin-2-one;
3-(3,5-dimethoxyphenyl)-5-hexyl-1,3-oxazolidin-2-one;
5-hexyl-3-(1,3-thiazol-4-yl)-1,3-oxazolidin-2-one;
2-fluoro-6-(5-hexyl-2-oxo-1,3-oxazolidin-3-yl)benzonitrile;
5-hexyl-3-(3-methoxy-4-methylphenyl)-1,3-oxazolidin-2-one;
[2-fluoro-4-(5-hexyl-2-oxo-1,3-oxazolidin-3-yl)phenyl]acetonitrile;
5-hexyl-3-(1H-pyrrolo[2,3-b]pyridin-3-yl)-1,3-oxazolidin-2-one;
5-hexyl-3-isothiazol-4-yl-1,3-oxazolidin-2-one;
5-hexyl-3-isothiazol-5-yl-1,3-oxazolidin-2-one;
3-(2-fluoro-3-methoxyphenyl)-5-hexyl-1,3-oxazolidin-2-one;
5-hexyl-3-(1-methyl-1H-pyrazol-4-yl)-1,3-oxazolidin-2-one;
5-hexyl-3-[4-(2-hydroxy-2-methylpropyl)phenyl]-1,3-oxazolidin-2-one;
5-hexyl-3-pyrazolo[1,5-b]pyridazin-3-yl-1,3-oxazolidin-2-one;
5-hexyl-3-(2-methoxyquinoxalin-6-yl)-1,3-oxazolidin-2-one;
5-hexyl-3-(2-methoxyquinazolin-6-yl)-1,3-oxazolidin-2-one;
3-(3-cyclopropylphenyl)-5-hexyl-1,3-oxazolidin-2-one;
3-(4-cyclopropylphenyl)-5-hexyl-1,3-oxazolidin-2-one;
5-hexyl-3-quinazolin-7-yl-1,3-oxazolidin-2-one;
5-hexyl-3-[2-(trifluoromethyl)quinolin-6-yl]-1,3-oxazolidin-2-one;
5-hexyl-3-quinazolin-6-yl-1,3-oxazolidin-2-one;
5-hexyl-3-thieno[2,3-c]pyridin-3-yl-1,3-oxazolidin-2-one;
5-hexyl-3-thieno[2,3-c]pyridin-2-yl-1,3-oxazolidin-2-one;
5-hexyl-3-(3-methoxyisoquinolin-7-yl)-1,3-oxazolidin-2-one;
5-hexyl-3-thieno[3,2-d]pyrimidin-6-yl-1,3-oxazolidin-2-one;
5-hexyl-3-(3-methoxycinnolin-7-yl)-1,3-oxazolidin-2-one;
5-hexyl-3-[4-(4H-1,2,4-triazol-3-yl)phenyl]-1,3-oxazolidin-2-one;
5-hexyl-3-[1,2,4]triazolo[1,5-a]pyridin-6-yl-1,3-oxazolidin-2-one;
7-(5-hexyl-2-oxo-1,3-oxazolidin-3-yl)phthalazin-1(2H)-one;
3-(2-cyclopropyl[1,2,4]triazolo[1,5-a]pyridin-6-yl)-5-hexyl-1,3-oxazolidin-2-one;
3-(4-amino-3-fluorophenyl)-5-hexyl-1,3-oxazolidin-2-one;
3-(4-cyclohexylphenyl)-5-hexyl-1,3-oxazolidin-2-one;
2-fluoro-4-(5-hexyl-2-oxo-1,3-oxazolidin-3-yl)benzonitrile;
3-(3-fluoro-5-methoxyphenyl)-5-hexyl-1,3-oxazolidin-2-one;
5-decyl-3-phenyl-1,3-oxazolidin-2-one;
5-butyl-3-phenyl-1,3-oxazolidin-2-one;
5-octyl-3-phenyl-1,3-oxazolidin-2-one;
5-but-3-en-1-yl-3-phenyl-1,3-oxazolidin-2-one;
5-(isobutoxymethyl)-3-phenyl-1,3-oxazolidin-2-one;
5-pentyl-3-phenyl-1,3-oxazolidin-2-one;
5-[(4-tert-butylphenoxy)methyl]-3-phenyl-1,3-oxazolidin-2-one;
5-{[(2-ethylhexyl)oxy]methyl}-3-phenyl-1,3-oxazolidin-2-one;
5-(butoxymethyl)-3-phenyl-1,3-oxazolidin-2-one;
(5R)-5-[2-(4-tert-butylphenyl)ethyl]-3-(3,4-dimethoxyphenyl)-1,3-oxazolidin-2-one;
(5R)-5-[2-(4-tert-butylphenyl)ethyl]-3-(4-chloro-3-methoxyphenyl)-1,3-oxazolidin-2-one;
3-{(5R)-542-(4-tert-butylphenyl)ethyl]-2-oxo-1,3-oxazolidin-3-yl) benzonitrile;
(5R)-5-[2-(4-tert-butylphenyl)ethyl]-3-phenyl-1,3-oxazolidin-2-one;
(5R)-5-[2-(4-tert-butylphenyl)ethyl]-3-isothiazol-5-yl-1,3-oxazolidin-2-One;
(5R)-5-[2-(4-tert-butylphenyl)ethyl]-3-thieno[2,3-c]pyridin-2-yl-1,3-oxazolidin-2-one;
(5R)-5-[2-(4-tert-butylphenyl)ethyl]-3-thieno[2,3-c]pyridin-3-yl-1,3-oxazolidin-2-one;
5-[(4-tert-butylphenoxy)methyl]-3-(3,4-dimethoxyphenyl)-1,3-oxazolidin-2-one;
3-{5-[(4-tert-butylphenoxy)methyl]-2-oxo-1,3-oxazolidin-3-yl}benzonitrile;
5-[(4-tert-butylphenoxy)methyl]-3-(4-chloro-3-methoxyphenyl)-1,3-oxazolidin-2-one;
5-[(4-tert-butylphenoxy)methyl]-3-thieno[2,3-c]pyridin-2-yl-1,3-oxazolidin-2-one;
5-[(4-tert-butylphenoxy)methyl]-3-thieno[2,3-c]pyridin-3-yl-1,3-oxazolidin-2-one;
5-[(4-tert-butylphenoxy)methyl]-3-isothiazol-5-yl-1,3-oxazolidin-2-one;
5-[(4-tert-butylphenoxy)methyl]-3-phenyl-1,3-oxazolidin-2-one;
5-[(bipbenyl-4-yloxy)methyl]-3-phenyl-1,3-oxazolidin-2-one;
5-[(3-ethylphenoxy)methyl]-3-phenyl-1,3-oxazolidin-2-one;
3-phenyl-5-[(5,6,7,8-tetrahydronaphthalen-2-yloxy)methyl]-1,3-oxazolidin-2-one;
5-[(4-ethylphenoxy)methyl]-3-phenyl-1,3-oxazolidin-2-one;
3-phenyl-5-[(4-propylphenoxy)methyl]-1,3-oxazolidin-2-one;
5-[(3-chloro-4-fluorophenoxy)methyl]-3-phenyl-1,3-oxazolidin-2-one;
5-[(4-isopropylphenoxy)methyl]-3-phenyl-1,3-oxazolidin-2-one;
3-phenyl-5-[4-(trifluoromethyl)phenoxy]methyl}-1,3-oxazolidin-2-one;
5-{[4-(1,1-dimethylpropyl)phenoxy]methyl}-3-phenyl-1,3-oxazolidin-2-one;
5-[(3-chlorophenoxyl)methyl]-3-phenyl-1,3-oxazolidin-2-one;
3-phenyl-5-[(3-propylphenoxy)methyl]-1,3-oxazolidin-2-one;
ethyl 4-[(2-oxo-3-phenyl-1,3-oxazolidin-5-yl)methoxy]benzoate;
5-[(4-bromophenoxy)methyl]-3-phenyl-1,3-oxazolidin-2-one;
3-phenyl-5-[(3,4,5-trimethylphenoxy)methyl]-1,3-oxazolidin-2-one;
5-[(2,3-dihydro-1H-inden-5-yloxy)methyl]-3-phenyl-1,3-oxazolidin-2-one;
5-[(3,5-dimethylphenoxy)methyl]-3-phenyl-1,3-oxazolidin-2-one;
5-[(4-ethoxyphenoxy)methyl]-3-phenyl-1,3-oxazolidin-2-one;
ethyl 3-[(2-oxo-3-phenyl-1,3-oxazolidin-5-yl)methoxy]benzoate;
5-[(3,5-dichlorophenoxy)methyl]-3-phenyl-1,3-oxazolidin-2-one;
3-phenyl-5-[3-(trifluoromethyl)phenoxy]methyl}-1,3-oxazolidin-2-one;
3-phenyl-5-[(4-propoxyphenoxy)methyl]-1,3-oxazolidin-2-one;
3-phenyl-5-[(5,6,7,8-tetrahydronaphthalen-1-yloxy)methyl]-1,3-oxazolidin-2-one;
5-[(4-fluorophenoxy)methyl]-3-phenyl-1,3-oxazolidin-2-one;
5-[(biphenyl-3-yloxy)methyl]-3-phenyl-1,3-oxazolidin-2-one;
5-[(2,3-difluorophenoxy)methyl]-3-phenyl-1,3-oxazolidin-2-one;
5-[(4-benzoylphenoxy)methyl]-3-phenyl-1,3-oxazolidin-2-one;
5-[(4-methoxyphenoxy)methyl]-3-phenyl-1,3-oxazolidin-2-one;
3-phenyl-5-[(2-propylphenoxy)methyl]-1,3-oxazolidin-2-one;
5-{[3-(dimethylamino)phenoxy]methyl}-3-phenyl-1,3-oxazolidin-2-one;
5-[(1-benzofuran-6-yloxy)methyl]-3-phenyl-1,3-oxazolidin-2-one;
5-[(4-phenoxyphenoxy)methyl]-3-phenyl-1,3-oxazolidin-2-one;
5-[(3-methylphenoxy)methyl]-3-phenyl-1,3-oxazolidin-2-one;
5-[(3,5-difluorophenoxy)methyl]-3-phenyl-1,3-oxazolidin-2-one;
5-(phenoxymethyl)-3-phenyl-1,3-oxazolidin-2-one;
5-[(2-chlorophenoxy)methyl]-3-phenyl-1,3-oxazolidin-2-one;
5-[(3-methoxyphenoxy)methyl]-3-phenyl-1,3-oxazolidin-2-one;
5-{[3-(diethylamino)phenoxy]methyl}-3-phenyl-1,3-oxazolidin-2-one;
5-[(1-naphthyloxy)methyl]-3-phenyl-1,3-oxazolidin-2-one;
5-[(2,4-dichlorophenoxy)methyl]-3-phenyl-1,3-oxazolidin-2-one;
5-[(4-cyclopentylphenoxy)methyl]-3-phenyl-1,3-oxazolidin-2-one;
5-[(3-tert-butylphenoxy)methyl]-3-phenyl-1,3-oxazolidin-2-one;
5-[(3,4-dichlorophenoxy)methyl]-3-phenyl-1,3-oxazolidin-2-one;
5-[(3-ethynylphenoxy)methyl]-3-phenyl-1,3-oxazolidin-2-one;
3-phenyl-5-{[3-(trifluoromethoxy)phenoxy]methyl}-1,3-oxazolidin-2-one;
5-[(3,5-dimethoxyphenoxy)methyl]-3-phenyl-1,3-oxazolidin-2-one;
5-[(4-isopropyl-3-methylphenoxy)methyl]-3-phenyl-1,3-oxazolidin-2-one;
5-{[4-fluoro-3-(trifluoromethyl)phenoxy]methyl}-3-phenyl-1,3-oxazolidin-2-one;
5-[(3-chloro-4-methylphenoxy)methyl]-3-phenyl-1,3-oxazolidin-2-one;
3-phenyl-5-[(quinolin-5-yloxy)methyl]-1,3-oxazolidin-2-one;
5-[(4-bromo-2-fluorophenoxy)methyl]-3-phenyl-1,3-oxazolidin-2-one;
5-[(4-bromo-3-fluorophenoxy)methyl]-3-phenyl-1,3-oxazolidin-2-one;
5-[(4-tert-butyl-2-methylphenoxy)methyl]-3-phenyl-1,3-oxazolidin-2-one;
3-phenyl-5-[4-(trifluoromethoxy)phenoxy]methyl}-1,3-oxazolidin-2-one;
5-[(3-aminophenoxy)methyl]-3-phenyl-1,3-oxazolidin-2-one;
5-[4-(3-oxobutyl)phenoxy]methyl}-3-phenyl-1,3-oxazolidin-2-one;
5-[(4-chloro-3-fluorophenoxy)methyl]-3-phenyl-1,3-oxazolidin-2-one;
5-[(3-tern-butyl-4-methoxyphenoxy)methyl]-3-phenyl-1,3-oxazolidin-2-one;
5-{[3-fluoro-5-(trifluoromethyl)phenoxy]methyl}-3-phenyl-1,3-oxazolidin-2-one;
ethyl 3-{4-[(2-oxo-3-phenyl-1,3-oxazolidin-5-yl)methoxy]phenyl}propanoate;
5-[(1,3-benzodioxol-5-yloxy)methyl]-3-phenyl-1,3-oxazolidin-2-one;
5-[(4-chloro-2-fluorophenoxy)methyl]-3-phenyl-1,3-oxazolidin-2-one;
5-[(3-phenoxyphenoxy)methyl]-3-phenyl-1,3-oxazolidin-2-one;
5-[(4-chloro-2-methoxyphenoxy)methyl]-3-phenyl-1,3-oxazolidin-2-one;
methyl 3-chloro-4-[(2-oxo-3-phenyl-1,3-oxazolidin-5-yl)methoxy]benzoate;
5-[(2,5-difluorophenoxy)methyl]-3-phenyl-1,3-oxazolidin-2-one;
5-[(3-benzoylphenoxy)methyl]-3-phenyl-1,3-oxazolidin-2-one;
5-[(isoquinolin-7-yloxy)methyl]-3-phenyl-1,3-oxazolidin-2-one;
5-[(4-anilinophenoxy)methyl]-3-phenyl-1,3-oxazolidin-2-one;
3-phenyl-5-[(3,4,5-trifluorophenoxy)methyl]-1,3-oxazolidin-2-one;
5-{[2-chloro-3-(trifluoromethyl)phenoxy]methyl}-3-phenyl-1,3-oxazolidin-2-one;
5-[(2,4-difluorophenoxy)methyl]-3-phenyl-1,3-oxazolidin-2-one;
3-phenyl-5-[(3,4,5-trimethoxyphenoxy)methyl]-1,3-oxazolidin-2-one;
5-[(2,5-dichlorophenoxy)methyl]-3-phenyl-1,3-oxazolidin-2-one;
3-phenyl-5-[(quinolin-8-yloxy)methyl]-1,3-oxazolidin-2-one;
5-[(4-acetyl-2-methylphenoxy)methyl]-3-phenyl-1,3-oxazolidin-2-one;
5-[(2-chloro-4-methoxyphenoxy)methyl]-3-phenyl-1,3-oxazolidin-2-one;
5-{[(3-oxo-2,3-dihydro-1-benzofuran-6-yl)oxy]methyl}-3-phenyl-1,3-oxazolidin-2-one;
3-phenyl-5-[(quinolin-6-yloxy)methyl]-1,3-oxazolidin-2-one;
3-benzyl-5-hexyl-1,3-oxazolidin-2-one;
3-benzyl-5-hexyl-1,3-oxazolidin-2-one;
5-hexyl-3-(2-methoxybenzyl)-1,3-oxazolidin-2-one;
5-hexyl-3-(3-methoxybenzyl)-1,3-oxazolidin-2-one;
5-hexyl-3-(4-methoxybenzyl)-1,3-oxazolidin-2-one;
2-[(5-hexyl-2-oxo-1,3-oxazolidin-3-yl)methyl]benzonitrile;
3-[(5-hexyl-2-oxo-1,3-oxazolidin-3-yl)methyl]benzonitrile;
4-[(5-hexyl-2-oxo-1,3-oxazolidin-3-yl)methyl]benzonitrile;
5-hexyl-3-(2-methylbenzyl)-1,3-oxazolidin-2-one;
5-hexyl-3-(3-methylbenzyl)-1,3-oxazolidin-2-one;
5-hexyl-3-(4-methylbenzyl)-1,3-oxazolidin-2-one;
methyl 4-[(5-hexyl-2-oxo-1,3-oxazolidin-3-yl)methyl]benzoate;
3-(4-fluorobenzyl)-5-hexyl-1,3-oxazolidin-2-one;
5-hexyl-3[3-(trifluoromethyl)benzyl]-1,3-oxazolidin-2-one;
3-(2-chlorobenzyl)-5-hexyl-1,3-oxazolidin-2-one;
3-(3-chlorobenzyl)-5-hexyl-1,3-oxazolidin-2-one;
3-(4-tert-butylbenzyl)-5-hexyl-1,3-oxazolidin-2-one;
3-(3-fluorobenzyl)-5-hexyl-1,3-oxazolidin-2-one;
5-hexyl-3-[3-(trifluoromethoxy)benzyl]-1,3-oxazolidin-2-one;
5-hexyl-3-[2-(trifluoromethoxy)benzyl]-1,3-oxazolidin-2-one;
5-hexyl-3-[4-(trifluoromethoxy)benzyl]-1,3-oxazolidin-2-one;
5-hexyl-3-[2-(trifluoromethyl)benzyl]-1,3-oxazolidin-2-one;
methyl 3-[(5-hexyl-2-oxo-1,3-oxazolidin-3-yl)methyl]benzoate;
3-(biphenyl-4-ylmethyl)-5-hexyl-1,3-oxazolidin-2-one;
3-(2-fluorobenzyl)-5-hexyl-1,3-oxazolidin-2-one;
5-hexyl-3-{4-(trifluoromethyl)benzyl}-1,3-oxazolidin-2-one;
3-(4-chlorobenzyl)-5-hexyl-1,3-oxazolidin-2-one;
5-{[(4-tert-butylbenzyl)(methyl)amino]methyl}-3-phenyl-1,3-oxazolidin-2-one;
5-{[(4-tert-butylbenzyl)amino]methyl}-3-phenyl-1,3-oxazolidin-2-one; and
5-{[benzyl(methyl)amino]methyl}-3-phenyl-1,3-oxazolidin-2-one,
or a pharmaceutically acceptable salt of any of the foregoing compounds.

16. A pharmaceutical composition comprising a compound according to claim 4 in combination with a pharmaceutically acceptable carrier.

17. A method for treating a neurological or psychiatric disorder associated with glutamate dysfunction in a patient in need thereof comprising administering to the patient a therapeutically effective amount of a compound according to claim 1.

18. The method according to claim 17 wherein the neurological or psychiatric disorder associated with glutamate dysfunction is schizophrenia.
